(19) United States
(12) Patent Application Publication
(10) Pub. No.: US 2010/0297147 A1
(43) Pub. Date: Nov. 25, 2010

(54) COMPOSITIONS AND METHODS FOR MODULATING TLR14 ACTIVITY

(75) Inventors: Katherine Seidl, Lexington, MA (US); Ying Gao, East Brunswick, NJ (US); Lih-Ling Lin, Concord, MA (US); Aisling Dunne, Dublin (IE); William John McCormack, Dublin (IE)

Correspondence Address: LANDO & ANASTASI, LLP, ONE MAIN STREET, SUITE 1100, CAMBRIDGE, MA, 02142, US

(73) Assignees: WYETH, Madison, NJ (US); OPSONA THERAPEUTICS, LTD., Dublin (IE)

(21) Appl. No.: 12/446,200

(22) PCT Filed: Oct. 18, 2007
(86) PCT No.: PCT/US07/22256

§ 371 Date: Jul. 29, 2010

(30) Foreign Application Priority Data
Oct. 18, 2006 (GB) 0620695.7

(51) Int. Cl. A61K 39/395; C12N 5/079; A61K 31/713; A61K 38/17; C12Q 1/68; G01N 33/53; C07K 16/28; A61P 25/00

(52) U.S. Cl. 424/172.1; 435/375; 514/44 A; 514/17.7; 435/6; 435/7.21; 530/389.1; 530/387.9

(57) ABSTRACT

Methods and compositions for modulating neural cell function using antagonists of TLR14 are disclosed. In particular, methods for treating, preventing and/or diagnosing TLR14-associated neurodegenerative conditions and/or disorders are disclosed. Screening methods for evaluating TLR14 modulators, e.g., agonists and antagonists, are also disclosed.

### CROSS REFERENCE TO RELATED APPLICATIONS

This application claims priority from British Application No. GB 0620695.7, entitled “Composition and Methods for the Treatment of Neurodegenerative Disease,” filed on October 18, 2006, which is incorporated herein by reference in its entirety.

### BACKGROUND

Toll-like Receptors (TLRs) are a group of highly conserved molecules that initiate the innate immune response to pathogens by recognizing structural motifs expressed by microbes. TLR receptors recognize a range of ligands and activate a series of signaling pathways that lead to the induction of immune and inflammatory genes.

Members of the TLR family are characterized by a cytosolic domain termed the Toll-IL-IR (TIR) domain and an extracellular region consisting of a series of leucine rich repeats. Activation of toll-like receptors during infection leads to the expression of a large number of pro-inflammatory proteins, such as inducible cyclooxygenase, adhesion molecules, chemokines, and the activation of immune effector cells. This coordinated response is designed to clear invading pathogens, however, in many instances bacterial products activate an uncontrolled network of host derived mediators which can lead to serious health conditions, including sepsis, multi-organ failure, cardiovascular collapse and eventually death.

Several human TLRs have been identified to date. TLR4, the first TLR to be discovered, is essential for the response to bacterial lipopolysaccharide (LPS) (Poltorak, A. et al. Science 282:2085-2088 (1998); Qureshi, S. T. et al. J. Exp. Med. 189:615-625 (1999)). TLR2 couples with TLRs 1 and 6 to recognize diacyl- and triacyl-lipopeptides, respectively. TLR5 recognizes and responds to bacterial flagellin (Hayashi, F. et al. Nature 410, 1099-1103 (2001)) and TLR9 is required for recognition of unmethylated CpG motifs, which are present in bacterial DNA (Hemmi, H. et al. Nature 408, 740-745 (2001)). TLRs 11, 12 and 13 have been described in mice, but no human ortholog has been identified to date (Zhang, D. et al. Science 303, 1522-1526 (2004); Tabeta, K. et al. PNAS 101, 3516-3521 (2004)). The amino acid and nucleotide sequences for human and mouse TLR14 have also been identified (WO 06/111946). Stimulation of most TLRs with the corresponding ligands leads to activation of the transcription factor NFkB, as well as the mitogen-activated protein kinases (MAPKs), p38, c-jun N terminal kinase (JNK) and p42/p44.

The activation of NFκB appears to be dependent on MyD88, a cytoplasmic TIR domain-containing adapter protein (Hemmi, H. et al. Nature Immunol. 3, 196-200 (2002); Adachi, O. et al. Immunity 9, 143-150 (1998); Takeuchi, O. et al. J. Immunol. 164, 554-557 (2000)). MyD88 seems to act as an adapter protein for the entire TLR family with the exception of TLR3, which recruits the adapter protein TRIF (Yamamoto, M. et al. J Immunol. 169, 6668-72 (2002)). In addition to activating NFκB, TRIF is also required for the induction of genes dependent on the transcription factor Interferon Regulatory Factor 3 (IRF3) utilized by both TLR3 and TLR4 MyD88 independent pathways (Kaisho, T. et al. (2001) J. Immunol. 166, 5688-5694). This pathway is referred to as the MyD88-independent pathway and has been shown to be important for evading pathogens of viral origin(Servant, M. J. et al. J. Biochem. Pharmacol. 64, 985-992 (2002)). Another TIR adapter protein, MyD88 Adapter-like (Mal, also known as TIRAP) is involved in the MyD88 dependent pathway (Fizgerald, K. A. et al. Nature 413, 78-83 (2001); Horng, T. et al. Nature Immunol. 2, 835-841 (2001)) and is required specifically for TLR2 and TLR4 mediated signaling (Yamamoto, M. et al. Nature 420, 324-329 (2002); Horng, T. et al. Nature 420, 329-33 (2002)).

Modulation of TLR proteins has been shown to be useful in counteracting the harmful effects of overactive immune responses. However, the need still exists for identifying novel activities associated with members of the TLR family, such as TLR14, and for using modulators of TLR proteins to reduce the activity of these proteins and alleviate symptoms of TLR-associated conditions.

### SUMMARY

The present invention is based, at least in part, on the discovery that Toll-like Receptor 14 (TLR14) binding agents, e.g., antibodies that bind to the extracellular domain of TLR14 protein, reverse myelin-induced inhibition of neurite outgrowth in vitro. Applicants also show that TLR14 mRNA and protein are expressed in selected regions of the murine brain, such as hippocampus, substantia nigra, basal ganglia, and cerebral and cerebellar cortex. Elevated TLR14 mRNA expression has been detected in the brain of patients afflicted with Alzheimer's and Parkinson's Disease. Co-immunoprecitation studies further revealed that TLR14 interacts with modulators of neuronal growth and/or activity, such as Nogo-66 receptor 1 (NgR) and neurotrophin receptor (p75^{NTR}). These findings implicate TLR14 in the regulation of neuronal activity and/or growth. The neuronal activities of TLR14 may be mediated by an interaction through NgR1 and/or p75^{NTR}. Thus, the present invention provides, in part, methods and compositions for modulating TLR14 function (e.g., TLR14 neural function) using TLR14 binding agents, e.g., antagonists of TLR14 function. In particular, methods for treating, preventing and/or diagnosing TLR14-associated conditions and/or disorders (e.g., TLR14-associated neurodegenerative conditions and disorders) are disclosed. Screening methods for evaluating TLR14 modulators, e.g., agonists and antagonists of TLR14 function or expression, are also disclosed.

Accordingly, in one aspect, the invention features a method of modulating a function (e.g., modulating one or more biological activities of TLR14) in a TLR14-responsive cell and/or tissue (e.g., a neural (e.g., a neuronal or glial) cell or tissue). The method includes contacting the TLR14-responsive cell and/or -tissue with a TLR14 modulator, e.g., a TLR14-binding agent, (e.g., an antagonist of human TLR14 activity or expression), in an amount sufficient to modulate the function of the TLR14-responsive cell or tissue (or the biological activity of TLR14 in the cell or tissue). In one embodiment, the contacting step can be effected in vitro, e.g., in a cell lysate or in a reconstituted system. Alternatively, the subject method can be performed on cells in culture, e.g., in vitro or ex vivo. For example, cells (e.g., purified or recombinant cells) can be cultured in vitro and the contacting step can be effected by adding the TLR14 modulator to the culture medium. Typically, the TLR14-responsive cell is a mammalian cell, e.g., a human cell. In some embodiments, the TLR14-responsive cell is a neural cell (e.g., a neuronal cell (e.g., a dopaminergic, cholinergic, cortical, cerebellar, hippocampal, spinal cord neuronal cell and/or a glial cell (e.g., microglia, astrocytes and oligodendrocytes)), or a population thereof. In other embodiments, the method can be performed on cells present in a subject, e.g., as part of an in vivo protocol, or in an animal subject (including, e.g., a human subject, or an in vivo animal model, such as a CNS inflammatory model. The in vivo protocol can be therapeutic or prophylactic, and the inflammatory model can be, for example, an EAE model, a lymphocytic meningeal encephalitis model, or a genetically modified model (e.g., an animal model having overexpressed TLR14, or a mutation or deletion in a TLR receptor). For in vivo methods, the TLR14 modulator, alone or in combination with another agent, can be administered to a subject suffering from a TLR14-associated neurodegenerative condition and/or disorder, in an amount sufficient to modulate, one or more TLR14 activities or functions in the subject.

In some embodiments, the amount or dosage of the TLR14 modulator that is administered can be determined prior to administration by testing in vitro or ex vivo, the amount of TLR14 modulator required to alter, e.g., decrease or inhibit, one or more of TLR14 activities (e.g., one or more TLR14 biological activities described herein). Optionally, the in vivo method can include the step(s) of identifying (e.g., evaluating, diagnosing, screening, and/or selecting) a subject having, or at risk of having, one or more symptoms associated with the disorder or condition.

In certain embodiments where inhibition, reduction or diminution of one or more TLR14 biological activities is desired, the TLR14-responsive cell and/or tissue is contacted with a TLR14 antagonist, e.g., by administering the TLR14 antagonist to the subject. In one embodiment, the TLR14 antagonist interacts with, e.g., binds to, a TLR14 polypeptide or mRNA, and reduces or inhibits one or more TLR14 activities. Typically, the TLR14 antagonized is a mammalian TLR14 (or a functional variant thereof), e.g., human TLR14 or murine TLR14. In certain embodiments, the TLR14 antagonized includes the human or murine TLR14 sequence comprising the amino acid sequence shown in FIG. 2 or 4 (SEQ ID NO:2 or SEQ ID NO:4, respectively), or a portion thereof, and/or a sequence substantially homologous thereto, or encoded by a nucleotide sequence shown in FIGS. 1 and 3 (SEQ ID NO:1 or SEQ ID NO:3, respectively), and/or a sequence substantially homologous thereto). In other embodiments, the antagonist may interfere with (e.g., reduce or inhibit) an interaction between TLR14 and other neuronal modulators, such as mammalian, e.g., human and murine, NgR1 and/or p75^{NTR} (e.g., a human and murine NgR1 and/or p75^{NTR} comprising the amino acid sequence shown in FIGS. 22B and 23B, or FIGS. 24B and 25B, respectively, or a portion thereof; or a sequence substantially homologous thereto; or encoded by a nucleotide sequence shown in FIGS. 22A and 23A, or FIGS. 24A and 25A, respectively, or a sequence substantially homologous thereto).

According to one embodiment, TLR14 modulators, including TLR14 binding agents (e.g., TLR14 antagonists) bind to TLR14 with high affinity, e.g., with an affinity constant of at least about 10⁷ M⁻¹, typically about 10⁸ M⁻¹, and more typically, about 10⁹ M⁻¹ to10¹⁰ M⁻¹ or stronger; and modulate, e.g., reduce and/or inhibit, one or more TLR14 biological activities in a TLR14 responsive cell and/or tissue (e.g., a neural cell or population of neural cells). The neural cell or population thereof can be, e.g., a neuronal cell, including a dopaminergic, cholinergic, cortical, cerebellar, hippocampal, spinal cord neuronal cell and/or a glial cell, including microglia, astrocytes and oligodendrocytes. Exemplary TLR14 activities that can be modulated, e.g., inhibited or reduced, using the methods and compositions of the invention include, but are not limited to, one or more of the following: (i) modulation of NgR1 binding binding or activity; (iii) modulation of p75^{NTR} binding or activity; (iii) modulation of endotoxin signaling, e.g., modulation of one or more endotoxin- (e.g., LPS)-mediated inflammatory signals in the brain (e.g., modulation of one or more of: endotoxin binding to TLR14, IxB degradation, phosphorylation of p38, pro-inflammatory cytokine or chemokine secretion, induction of nitric oxide production in, e.g., astrocytes, or modulation of neuronal viability and/or gliosis); and/or (iv) modulation of one or more of: neuronal cell survival, differentiation and/or neurite outgrowth.

As shown in the appended Examples, contacting of TLR14-expressing cerebellar granule neurons with TLR14 binding antibodies reversed myelin-induced inhibition of neurite outgrowth in vitro. Thus, without being bound by theory, Applicants believe that TLR14 acts as a negative regulator of neurite outgrowth, and thus inhibition of TLR14 activity may be beneficial in treating or preventing disorders or conditions, and/or alleviating symptoms, associated with brain injury and/or brain disorders (e.g., neurodegenerative disorders).

In one embodiment, the TLR14 modulator is an antibody molecule against TLR14. The antibody molecule can be a monoclonal or single specificity antibody, or an antigen-binding fragment thereof (e.g., an Fab, F(ab′)₂, Fv, a single chain Fv fragment, a shark antibody, or a camelid variant) that binds to TLR14, e.g., a mammalian (e.g., human, TLR14 (or a functional variant thereof)). Typically, the antibody molecule is a human, humanized, chimeric, camelid, or in vitro generated antibody to human TLR14 (or functional fragment thereof). Typically, the antibody molecule inhibits, reduces or neutralizes one or more activities of TLR14 (e.g., one or more biological activities of TLR14 as described herein). In one embodiment, the antibody molecule binds to TLR14 polypeptide, or a portion thereof, e.g., the extracellular domain of TLR14 (e.g., about amino acids 1 to 695 or 696, or a fragment thereof, e.g., about amino acids 85 to 103, or 509 to 526, of FIG. 2 (SEQ ID NO:2). In other embodiments, the antibody molecule binds to a Leucine-rich domain of TLR14, e.g., a domain located at about amino acids 26 to 58, 82 to 105, 106 to 129, 130 to 153, 154 to 177, 178 to 201, 202 to 227, 228 to 251, 252 to 275, 276 to 299, 300 to 323, 324 to 347, or 359 to 315, of FIG. 2 (SEQ ID NO:2). In yet another embodiment, the antibody molecule binds to a fibronectin-type III domain (FN3) located at about amino acids 589 to 671, of FIG. 2 (SEQ ID NO:2), and/or the RGD integrin binding site located at about amino acids 640 to 660, 645 to 655, or 652 to 654, of FIG. 2 (SEQ ID NO:2). Human TLR14 protein may include a leader sequence about 20-21 amino acids long corresponding to about amino acids 1 to 20 of SEQ ID NO:2. In yet other embodiments, the antibody molecule binds to an intracellular domain of TLR14, e.g., at about amino acids 720 to 811, or a portion thereof (e.g., about amino acids 796 to 811, of FIG. 2 (SEQ ID NO:2)). In yet other embodiments, the antibody molecule competes for binding to TLR14 with any of antibodies 0540 (A), 0541 (B), and 0547 (C) describes herein (e.g., the antibody molecule binds to an epitope on TLR14 that is the same or substantially similar to the one recognized by antibodies 0540 (A), 0541 (B), and 0547 (C)). In other embodiments, the antibody molecule binds to an epitope as described herein and inhibits, reduces or neutralizes one or more activities of TLR14.

In certain embodiments, the antibody molecule binds to a TLR14-associated polypeptide, e.g., NgR1(e.g., a human NgR1); or p75^{NTR} (e.g., a human p75^{NTR}). For example, the antibody molecule binds to an NgR1 or a p75^{NTR} comprising an amino acid sequence identical to a mammalian, e.g., human NgR1 or p75^{NTR}, as shown in FIG. 22B (SEQ ID NO:8) or FIG. 24B (SEQ ID NO:12), respectively), or a sequence substantially homologous thereto, and inhibits, reduces or neutralizes one or more activities of TLR14.

The antibody molecule can be full-length (e.g., can include at least one, and typically two, complete heavy chains, and at least one, and typically two, complete light chains) or can be an antigen-binding fragment (e.g., a Fab, F(ab′)₂, Fv or a single chain Fv fragment). In yet other embodiments, the antibody molecule has a heavy chain constant region chosen from, e.g., the heavy chain constant regions of IgGi, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of Fc receptor binding, antibody glycosylation, the number of cysteine residues, effector cell function, and/or complement function).

In other embodiments, the TLR14 antagonist is a full length, or a fragment of a TLR14 polypeptide. For example, the antagonist can be a soluble form of TLR14-associated protein (e.g., a soluble form of mammalian (e.g., human) TLR14; e.g., a soluble form of an extracellular domain of mammalian (e.g., human) TLR14). For example, the TLR14 antagonist can include about amino acids 1 to 695, or a fragment thereof, of FIG. 2 (SEQ ID NO:2).

A soluble form of TLR14 can be used alone or functionally linked (e.g., by chemical coupling, genetic or polypeptide fusion, non-covalent association or otherwise) to a second moiety, e.g., an immunoglobulin Fc domain, serum albumin, pegylation, a GST, Lex-A or an MBP polypeptide sequence. The fusion proteins may additionally include a linker sequence joining the first moiety, e.g., a soluble TLR14, to the second moiety. In other embodiments, additional amino acid sequences can be added to the N- or C-terminus of the fusion protein to facilitate expression, steric flexibility, detection and/or isolation or purification. For example, a soluble form of a TLR14 can be fused to a heavy chain constant region of the various isotypes, including: IgG1, IgG2, IgG3, IgG4, IgM, IgA1, IgA2, IgD, and IgE). Typically, the fusion protein can include the extracellular domain of a human TLR14 (or a sequence homologous thereto), and, e.g., fused to, a human immunoglobulin Fc chain, e.g., human IgG (e.g., human IgG1 or human IgG2, or a mutated form thereof). The Fc sequence can be mutated at one or more amino acids to reduce effector cell function, Fc receptor binding and/or complement activity.

The antibody molecules and/or soluble or fusion proteins described herein can be functionally linked (e.g., by chemical coupling, genetic fusion, non-covalent association or otherwise) to one or more other molecular entities, such as an antibody (e.g., a bispecific or a multispecific antibody), toxins, radioisotopes, cytotoxic or cytostatic agents, among others.

In another embodiment, the TLR14 modulator, e.g., the TLR14 binding agent inhibits the expression of nucleic acid encoding a TLR14. Examples of such TLR14 binding agents, e.g., TLR14 antagonists, include nucleic acid molecules, such as antisense molecules, ribozymes, RNAi, and triple helix molecules. These nucleic acids hybridize to a nucleic acid encoding a TLR14, or a transcription regulatory region thereof, and block or reduce mRNA expression of TLR14.

In in vivo embodiments, the TLR14 modulator, e.g., the TLR14 antagonist, can be administered to a subject having, or at risk of having, a TLR14-associated disorder and/or condition. In such embodiments, the subject is a mammal, e.g., a human, suffering from a TLR14-associated neurodegenerative condition and/or disorder. The neurodegenerative condition and/or disorder can be an acute neurodegenerative condition, or a chronic or progressive condition. Particular selected conditions or disorders that can be treated, or prevented, using the methods and compositions of the invention, include but are not limited to, a condition or disorder involving a CNS injury and/or axonal degeneration; Parkinson's disease; dementia, including but not limited to Alzheimer's disease and age-related dementia; Huntington's disease; amyotrophic lateral sclerosis (ALS); traumatic brain injury; spinal cord injury; multiple sclerosis; neuropathy associated with medical treatments such as chemotherapy; ischemia or ischemia-induced injury; and stroke. The neurodegenerative condition or disorder can also include infections and neuroinflammatory conditions, such as CNS conditions caused by bacterial infections (e.g., Staphilococous, Streptococous and Pneumococous) and encephalitis. In certain embodiments, the TLR14 modulator reduces or alleviates one or more symptoms in a subject associated with a neurodegenerative condition. In certain embodiments, the TLR14 modulators diminishes or stabilizes progression of a neurodegenerative disorder.

The TLR14 modulator can be administered to the subject alone or in combination with one or more agents (e.g., a second therapeutic agent or therapeutic modality), which are useful for treating TLR14 associated neurodegenerative disorders and/or conditions. In one embodiment, the second agent or therapeutic modality is a NgR1antagonist, a p75^{NTR} antagonist, a beta blocker, a hormone antagonist, an endothelin antagonist, a calcium channel blocker, a cytokine inhibitor, a statin, and/or an anti-inflammatory agent. In other embodiments, this second agent may be an agent otherwise useful for treating symptoms of neuronal disorders (e.g., donepezil (Aricept™) and rivastigmine tartrate (Exelon™)).

In yet another aspect, the invention features a method of treating or preventing (e.g., curing, suppressing, ameliorating, alleviating one or more symptoms, delaying or preventing the onset or progression of, or preventing recurrence or relapse of) a TLR14-associated condition and/or disorder (e.g., a TLR14-associated neurodegenerative condition and/or disorder), in a subject. The method includes administering to the subject a TLR14 modulator, e.g., a TLR14 binding agent (e.g., a TLR14 antagonist as described herein), in an amount sufficient to modulate, e.g., inhibit or reduce, one or more TLR14 biological activities in a TLR14-responsive cell and/or tissue, e.g., a neural cell and/or tissue (e.g., a biological activity as described herein), thereby treating or preventing the disorder or condition. The subject can be a mammal, e.g., a human, suffering from, for example, a TLR14-associated condition and/or disorder as described herein. In some embodiments, the amount or dosage of the TLR14 modulator administered can be determined, e.g., prior to administration to the subject, by testing in vitro or ex vivo the amount of TLR14 antagonist required to inhibit or reduce one or more of the aforesaid TLR14 biological activities. The method can, optionally, include the step(s) of identifying (e.g., evaluating, diagnosing, screening, and/or selecting) a subject at risk of having, or having, one or more symptoms associated with the TLR14-associated condition and/or disorder (e.g., a neurodegenerative condition and/or disorder as described herein).

The neurodegenerative condition and/or disorder can be an acute neurodegenerative condition, or a chronic or progressive condition. Particular selected conditions or disorders that can be treated, or prevented, using the methods and compositions of the invention, include but are not limited to, a condition or disorder involving a CNS injury and/or axonal degeneration; Parkinson's disease; Alzheimer's disease; Huntington's disease; amyotrophic lateral sclerosis (ALS); traumatic brain injury; spinal cord injury; multiple sclerosis; neuropathy associated with medical treatments such as chemotherapy; ischemia or ischemia-induced injury; and stroke. The neurodegenerative condition or disorder can also include infections and neuroinflammatory conditions, such as CNS conditions caused by bacterial infections (e.g., Staphilococous, Streptococous and Pneumococous) and encephalitis.

The TLR14 modulator can be administered to the subject alone or in combination with one or more agents (e.g., a second therapeutic agent or therapeutic modality), which are useful for treating TLR14 associated neurodegenerative disorders and/or conditions. In one embodiment, the second agent or therapeutic modality is a NgR1antagonist, a p75^{NTR} antagonist, a beta blocker, a hormone antagonist, an endothelin antagonist, a calcium channel blocker, a cytokine inhibitor, a statin, and/or an anti-inflammatory agent. In other embodiments, this second agent may be an agent otherwise useful for treating symptoms of neuronal disorders (e.g., donepezil (Aricept™) and rivastigmine tartrate (Exelon™)).

In yet another aspect, the invention provides a method of stimulating one or more of neurite outgrowth, survival, and/or differentiation of a neuronal cell (e.g., a dopaminergic, cholinergic, cortical, cerebellar, hippocampal and/or spinal cord neuronal cell). The method includes contacting the cell with a TLR14 modulator, e.g., a TLR14 binding agent as described herein (e.g., a TLR14 antagonist). The antagonist can be an inhibitor of activity and/or expression of TLR14. In one embodiment, the inhibitor is an extracellular antagonist of TLR14, e.g., an antibody molecule that interacts with the extracellular domain of TLR14. In other embodiments, the TLR4 modulator is intracellular antagonist of TLR14, e.g., the TLR14 antagonist is an inhibitor of transcription of TLR14 mRNA, e.g., RNAi. Typically, the TLR14 modulator is administered in an amount sufficient to interact with TLR14 and/or TLR14-associated proteins, e.g., NgR1 and/or p75^{NTR}. The contacting step can be effected in vitro, e.g., in culture, or in vivo, e.g., by administration to a subject, as described herein.

In yet another aspect, the invention provides a method of regenerating and/or repairing neural tissue in a subject. The method includes contacting the neural tissue with a TLR14 modulator, e.g., a TLR14 binding agent (e.g., a TLR14 antagonist). The antagonist can be an inhibitor of activity and/or expression of TLR14. In one embodiment, the inhibitor is an extracellular antagonist of TLR14, e.g., an antibody molecule that interacts with the extracellular domain of TLR14. In other embodiments, the TLR14 modulator is intracellular antagonist of TLR14, e.g., the TLR14 antagonist is an inhibitor of transcription of TLR14 mRNA, e.g., RNAi. In certain embodiments, the TLR14 modulator is administered in an amount sufficient to interact with TLR14 and/or TLR14-associated proteins, e.g., NgR1 and/or p75^{NTR}. The contacting step can be effected in vitro, e.g., in culture; or in vivo, e.g., by administration to a subject as described herein.

In yet another aspect, the invention provides a method of modulating, e.g., reducing or inhibiting, in a subject, an immune response associated with a neurodegenerative disease, or an endotoxin-mediated inflammation response in the brain. The method includes administering to the subject a TLR14 modulator, e.g., a TLR14 binding agent (e.g., a TLR14 antagonist). The antagonist can be an inhibitor of activity and/or expression of TLR14. In one embodiment, the inhibitor is an extracellular antagonist of TLR14, e.g., an antibody molecule that interacts with the extracellular domain of TLR14. In other embodiments, the TLR4 modulator is intracellular antagonist of TLR14, e.g., the TLR14 antagonist is an inhibitor of transcription of TLR14 mRNA, e.g., RNAi. Typically, the TLR14 modulator is administered in an amount sufficient to interact with (e.g., bind to and/or inhibit the association and/or activity of) TLR14 and/or TLR14-associated proteins, e.g., NgR1 or p75^{NTR}. In other embodiments, the TLR14 modulator is administered in an amount sufficient to interact with (e.g., bind to and/or inhibit the association and/or activity of) TLR14 and the endotoxin, e.g., LPS.

In another aspect, the invention provides TLR14 modulators, e.g., TLR14 binding agents (e.g., antagonists of TLR14 expression and/or activity in a neural cell and/or tissue). For example, TLR14 binding agents (e.g., anti-TLR14 antibodies, soluble TLR14 or fusion proteins thereof, can be identified and/or generated using the methods disclosed herein. Compositions, such as pharmaceutical compositions, that include the TLR14 modulators are also disclosed. It is noted that the compositions may additionally include a second therapeutic agent, e.g., a second therapeutic agent as described herein.

Packaged pharmaceutical compositions that include the TLR14 modulators for use in treating a neurodegenerative disorder or condition described herein are also encompassed by the present invention. Optionally, the packaged pharmaceutical composition is labeled and/or contains some instructions for use in treating a neurodegenerative disorder or condition described herein.

In another aspect, the invention features TLR14 binding agents, e.g., antibody molecules, variant molecules, small molecules, antisense nucleic molecules which interact with, or more preferably specifically bind to TLR14 polypeptides or fragments thereof, or nucleic acids encoding TLR14. In one embodiment, the antibody molecules or the variant molecules bind to a mammalian, e.g., human, TLR14 polypeptide or a fragment thereof. In one embodiment, the antibody molecule binds to TLR14 polypeptide, or a portion thereof, e.g., the extracellular domain of TLR14 (e.g., about amino acids 1 to 695 or 696, or a fragment thereof, e.g., about amino acids 85 to 103, or 509 to 526, of FIG. 2 (SEQ ID NO:2). In other embodiments, the antibody molecule binds to a Leucine-rich domain of TLR14, e.g., a domain located at about amino acids 26 to 58, 82 to 105, 106 to 129, 130 to 153, 154 to 177, 178 to 201, 202 to 227, 228 to 251, 252 to 275, 276 to 299, 300 to 323, 324 to 347, or 359 to 315, of FIG. 2 (SEQ ID NO:2). In yet another embodiment, the antibody molecule binds to a fibronectin-type III domain (FN3) located at about amino acids 589 to 671, of FIG. 2 (SEQ ID NO:2), and/or the RGD integrin binding site located at about amino acids 640 to 660, 645 to 655, or 652 to 654, of FIG. 2 (SEQ ID NO:2). Human TLR14 protein may include a leader sequence about 20-21 amino acids long corresponding to about amino acids 1 to 20 of SEQ ID NO:2. In yet other embodiments, the antibody molecule or the variant molecule binds to an intracellular domain of TLR14, e.g., at about amino acids 720 to 811, or a portion thereof (e.g., about amino acids 796 to 811, of FIG. 2 (SEQ ID NO:2)). In yet other embodiments, the antibody molecule or the variant molecule binds competes for binding to TLR14 with any of antibodies 0540 (A), 0541 (B), and 0547 (C) describes herein (e.g., the antibody molecule binds to an epitope on TLR14 that is the same or substantially similar to the one recognized by antibodies 0540 (A), 0541 (B), and 0547 (C)). In other embodiments, the antibody molecule or the variant molecule binds to an epitope as described herein and inhibits, reduces or neutralizes one or more activities of TLR14. In one embodiment, the antibody molecules or the variant molecules modulate, e.g., antagonize, one or more TLR14-associated activities, e.g., an activity as described herein.

In one aspect, the invention features a method of providing an antibody molecule or a variant molecule that specifically binds to a human TLR14 protein. The method includes: providing a human TLR14 protein or fragment thereof (e.g., an antigen that comprises at least a portion of the TLR14 protein as described herein); obtaining an antibody molecule or the variant molecule that specifically binds to the human TLR14 protein or fragment thereof; and evaluating if the antibody molecule or the variant molecule specifically binds to the human TLR14 protein, or evaluating efficacy of the antibody molecule or the variant molecule in modulating, e.g., inhibiting, the activity of the human TLR14 protein. The method can further include administering the antibody molecule to a subject, e.g., a human or non-human animal.

In another aspect, the invention features a method of evaluating, diagnosing, and/or monitoring the progression of, a TLR14 associated disorder, e.g., a neurodegenerative disorder (e.g., a disorder as described herein) in a test sample. The method includes evaluating the expression or activity of a nucleic acid or polypeptide chosen from TLR14 or a TLR14-associated gene, such that a difference in the level of the nucleic acid or polypeptide relative to a reference sample, e.g., a sample obtained from normal subject or prior to treatment, is indicative of the presence or progression of the disorder. In certain embodiments, the TLR14-associated nucleic acid or polypeptide is characterized by altered expression in response to TLR14. In certain embodiments, an increase in the level of TLR14 or a TLR14-associated gene in the test sample, relative to a reference sample, is associated with the diagnosis of TLR14 disorder where antagonism of TLR14 function is desirable (e.g., a neurodegenerative disorder as described herein). In other embodiments, a decrease in the level of a TLR14 or a TLR14-associated gene in the test sample, relative to a reference sample, is associated with the diagnosis of TLR14 disorder where agonism of TLR14 function is desirable.

In one embodiment, the evaluating step occurs in vitro or ex vivo. For example, a sample, e.g., a serum sample, is obtained from the subject.

In another embodiment, the evaluating step occurs in vivo. For example, by administering to the subject a detectably labeled agent that interacts with the TLR14, or TLR14 associated, nucleic acid or polypeptide, such that a signal is generated relative to the level of activity or expression of the nucleic acid or polypeptide.

In yet another aspect, the invention provides a method, or an assay, for identifying a compound, e.g., a test compound, that modulates TLR14 function The method, or the assay, includes: providing or identifying a test agent that interacts with, e.g., binds to, TLR14 or a TLR14-associated protein. The method can further include the step of evaluating a change in an activity of a neural tissue in the presence of the test agent, relative to a reference, e.g., a reference sample.

The test compound can be an antibody molecule; peptide; a soluble TLR14 or a fusion thereof; a variant molecule; a small molecule, e.g., a member of a combinatorial or natural product library; a nucleic acid; an antisense molecule; a ribozyme;an RNAi; a triple helix molecule; or any combination thereof. In one embodiment, the test compound modulates (e.g., decreases or increases) the activity or expression of a TLR14 polypeptide or nucleic acid. For example, the expression of the TLR14 nucleic acid can be modulated by, e.g., altering mRNA transcription and/or altering mRNA stability.

In certain embodiments, the evaluating step includes contacting one or more of a TLR14 (e.g., a TLR14 as described herein), or a nucleic acid encoding the TLR14, with the test compound; and evaluating a change in one or more activities of the TLR14 polypeptide or nucleic acid, in the presence of the test compound, relative to a predetermined level, e.g., a control sample without the test compound. The contacting step can be effected in vitro (in cultured cells, e.g., neural cells, or a reconstituted system) or in vivo (e.g., by administering the test compound to a non-human subject, e.g., an animal model having a CNS inflammatory disorder, e.g., EAE or a mutation in a TLR14 or a gene encoding a TLR14 associated protein, e.g., NgR1 and/or p75^{NTR}). The contacting step(s) and/or the administration of the test compound can be repeated.

In other embodiments, the change in an activity of the TLR14-responsive cell is evaluated by measuring a change, in the presence of the test compound, relative to a reference, e.g., a reference sample (e.g., a control sample not exposed to the test compound), in one or more of: (i) modulation of NgR1 binding or activity; (ii) modulation of p75^{NTR} binding or activity; (iii) modulation of endotoxin signaling, e.g., modulation of one or more endotoxin- (e.g., LPS-) mediated inflammatory signals, in the brain (e.g., modulation of one or more of: endotoxin binding to TLR14, IκB degradation, phosphorylation of p38, pro-inflammatory cytokine or chemokine secretion, induction of nitric oxide production in, e.g., astrocytes, or modulation of neuronal viability and/or gliosis; (iv) modulation of one or more of: neuronal cell survival, differentiation, neurite outgrowth and/or axonal regeneration; (v) monitoring the activation of one or more of: RhoA, PKC, CdC42, Racl, PAK1, ROCK, LIM-kinase, cofilin, actin, cAMP levels, ERK activation, NF-kB, transcriptional activation of a large family of immune genes such as TNFα, IL-1b, MCP-1, IL-12 or the production of NO; and/or (vi) a combination of (i) to (iv). In certain embodiments, a decrease in one or more of (i)-(vi) is indicative of an antagonist of TLR14 function, which may be a candidate for treatment of a neurodegenerative disorder where TLR14 antagonism is desirable.

In certain embodiments, an interaction between the test compound, the TLR14 or the TLR14-associated polypeptide, e.g., NgR1 or p75^{NTR} is evaluated. In certain embodiments, such interaction can be evaluated by detecting a change in the formation and/or stability of the complex between the test compound and TLR14 and/or TLR14-associated polypeptide. For example, binding, e.g., the formation of a complex, between TLR14 and NgR1 or p75^{NTR} in the absence or a presence of a test compound can be evaluated. Such interaction can be determined by detecting one or more of a change in the binding or physical formation of the complex itself, e.g., by biochemical detection, affinity based detection (e.g., Western blot, affinity columns), immunoprecipitation, fluorescence resonance energy transfer (FRET)-based assays, spectrophotometric means (e.g., circular dichroism, absorbance, and other measurements of solution properties); a change, e.g., increase or decrease, in signal transduction, e.g., LPS binding to TLR14, IiB degradation, phosphorylation of p38, pro-inflammatory cytokine or chemokine secretion, induction of nitric oxide production in, e.g., astrocytes, or modulation of neuronal viability and/or gliosis; and/or modulation of one or more of: neuronal cell survival, differentiation and/or neurite outgrowth.

In one embodiment, the test compound is identified and evaluated in the same or a different assay. For example, a test compound is identified in an in vitro or cell-free system, and evaluated in an animal model or a cell-based assay. Any order or combination of assays can be used. For example, a high throughput assay can be used in combination with an animal model or tissue culture.

In other embodiments, the method, or assay, includes providing a step based on proximity-dependent signal generation, e.g., a two-hybrid assay that includes a first fusion protein (e.g., a fusion protein comprising a TLR14 portion) and a second fusion protein (e.g., a fusion protein comprising a TLR14-associated polypeptide), and contacting the two-hybrid assay with a test compound under conditions wherein said two hybrid assay detects a change in the formation and/or stability of the complex, e.g., the formation of the complex initiates transcription activation of a reporter gene.

In yet another aspect, the invention provides a host cell comprising one or more nucleic acids encoding one or more of the TLR14 or TLR14-associated polypeptide constituents of the complex disclosed herein.

In another aspect, the invention provides a composition, e.g., a pharmaceutical composition, comprising a TLR14 modulator e.g., a TLR14 binding agent (e.g., a TLR14 antagonist) and a for treating a pharmaceutically acceptable carrier. The composition can include one or more additional agents suitable for combination therapy with a TLR14 modulator. The composition can be used to treat a subject, e.g., a patient, having a neurodegenerative disease.

In yet another aspect, the invention provides the use of a TLR14 modulator, e.g., a TLR14 binding agent (e.g., a TLR14 antagonist) in the preparation of a medicament for one or more of: (i) the treatment of a neurodegenerative disorder or condition, e.g., a disorder or condition as described herein; (ii) inducing one or more of: neuronal cell survival, differentiation, neurite and/or axonal outgrowth, and/or neuronal regeneration; or (iii) reducing or inhibiting endotoxin-mediated signaling in the brain.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

The terms “proteins” and “polypeptides” are used interchangeably herein.

“About” and “approximately” shall generally mean an acceptable degree of error for the quantity measured given the nature or precision of the measurements. Exemplary degrees of error are within 20 percent (%), typically within 10%, and more typically, within 5% of a given value or range of values. In the context of a range value for an amino acid or nucleotide sequence, the term “about” includes a range that differs by 1, 2, 3, 4 or 5 residues or nucleotides at one or both end points. For example, the phrase “about amino acids 9 to 22” of a sequence can include amino acid sequences, such as 7 to 23 and 11 to 20 of the amino acid sequence specified.

All publications, patent applications, patents, and other references mentioned herein are incorporated by reference in their entirety.

Other features, objects, and advantages of the invention will be apparent from the description and drawings, and from the claims.

### DESCRIPTION OF DRAWINGS

FIG. 1 shows the nucleotide sequence of human TLR14 (SEQ ID NO:1).

FIG. 2 shows the amino acid sequence of human TLR14 (SEQ ID NO:2).

FIG. 3 shows the nucleotide sequence of murine TLR14 (SEQ ID NO:3).

FIG. 4 shows the amino acid sequence of murine TLR14 (SEQ ID NO:4).

FIG. 5 shows a phylogenetic analysis of TLR14 and other leucine rich repeat containing proteins.

FIG. 6 shows a sequence alignment of TLR14 and Nogo-66 receptor 1 (NgR). Shaded areas indicate regions of identity.

FIG. 7A shows the crystal structure of NgR.

FIG. 7B shows the predicted structure of TLR14 generated using the automated Swiss-model homology-modeling pipeline available from expasy.org.

FIG. 8 shows homology between TLR14 and members of the Fibronectin domain containing family of proteins. Fibronectin leucine rich transmembrane protein (FLRT (e.g., FLRT 1-3)) contains LRR (Leucine-rich repeat domain), FN3 (fibronectin type III domain), and transmembrane domains (TM). Bold font indicates a FN3 domain. Bold underlined font indicates an RGD integrin binding site. Double lined open box indicates a transmembrane domain. LRR domains are amino acids 26-58 (LRRNT); amino acids 82-105 (LRR TYP); amino acids 106-129 (LRR TYP); amino acids 130-153 (LRR TYP); amino acids 154-177 (LRR TYP); amino acids 178-201 (LRR TYP); amino acids 202-227 (LRR); amino acids 228-251 (LRR TYP); amino acids 252-275 (LRR TYP); amino acids 276-299 (LRR TYP); amino acids 300-323 (LRR TYP); amino acids 324-347 (LRR TYP); and amino acids 359-415 (LRRCT). The sequence shown is human TLR14 (SEQ ID NO: 2). Human TLR14 protein may include a leader sequence about 20-21 amino acids long corresponding to about amino acids 1 to 20 of SEQ ID NO:2. The domain were predicted according to SMART software as described in Schultz et al. (1998) Proc Natl Acad Sci USA 95:5857-5864 and Letunic et al. (2006) Nucleic Acid Res 34: D257-D260.

FIG. 9 is a table showing additional proteins that contain LRR and FN3 domains. Leucine rich and fibronectin containing (LRFN); neuronal leucine-rich repeat protein (NLRR/LRRN); photoreceptor-associated LRR superfamily (Pal); SLIT-like 2 (Homo Sapiens Vasorin) (SLITL). Other abbreviations: IgC2 (immmunoglobulin domain of C2 class), EGF (epiderimal growth factor domain), TyrK (tyrosin kinase domain).

FIG. 10 shows an mRNA expression profile for human TLR14 obtained from SymAtlas (Su, A. et al. (2002) PNAS 99(7):4465-70).

FIG. 11 is a scatter plot showing expression profiling of TLR14 in brain tissue.

FIG. 12 is a bar graph showing elevated TLR14 mRNA levels in Alzheimer's and Parkinson's disease patients. FIG. 12A shows TLR14 mean±standard deviation expression levels for normal, Alzheimer's, and Parkinson's brain samples measured by GeneChip™ in the Genelogic expression database. FIG. 12B shows individual measurements used for (A). FIG. 12C is a tabular summary of the data present in FIGS. 12A and 12B. “Frequency database” indicates the overall frequency of detection for TLR14. “% present” indicates fractions of normal, Alzheimer's, and Parkinson's samples in which TLR14 was detected.

FIG. 13 shows the epitopes on human TLR14 recognized by the anti-TLR14 antibodies 0540 (A), 0541 (B), and 0547 (C). The transmembrane domain is shown by an open box.

FIGS. 14A-14D are immunoblots showing anti-TLR14 0547 antibody peptide competition assays. Cell types used were CHO-K1 (1), TLR14/CHO-K1 (2), CHO-K1 (3), astrocyte (4), E18 CN (cortical neuron) (5), P4 CGN (cerebellar granule neuron) (6 and 7), P7 Cb (cerebellum lysate) (8), DIV (days in vitro). FIGS. 14A and 14B show TLR14 and NgR1 immunoblots performed using 0547 and NgR1 antibody, respectively, in the absence of a competing peptide. FIGS. 14C and 14D shown TLR14 and NgR1 immunoblots performed using 0547 and anti-NgR1 antibody, respectively, in the presence of the 0547 competing peptide. Arrows indicate the band corresponding to NgR.

FIGS. 15A-15C are scatter plots showing TLR14 expression in the brains of wild type (wt) mice. Three mice were analyzed (wt3-wt6). FIG. 15A shows data generated using the 0547 anti-TLR14 antibody. FIG. 15B shows data generated using the 0541 anti-TLR14 antibody. FIG. 15C shows data generated using an isotypic control antibody.

FIG. 16 is an immunoblot showing TLR14 (top), NgR1(second), p75 (third), and actin (bottom) expression. Star indicates bands corresponding to NgR1 and p75. Actin was used as a loading control. NgR, p75^{NTR}, or TLR14 construct was expressed alone or in combinations in CHO-K1 cells. Lysates were analyzed by Western blot with an antibody to TLR14, NgR, p75^{NTR}, or actin as indicated.

FIG. 17 is an immunoblot showing TLR14, NgR, and p75 following immunoprecipitation using 0547 antibody. Star indicates a band corresponding to NgR. Epsilon indicates a band corresponding to p75.

FIG. 18 is an immunoblot showing NgR, TLR14, p75 following immunoprecipitation using anti-NgR1 antibody. NgR, p75^{NTR}, or TLR14 construct was expressed alone or in combinations in CHO-K1 cells.

FIG. 19 is an immunoblot showing NgR, TLR14, p75 following immunoprecipitation using anti-p75 antibody.

FIG. 20 a bar graph showing neurocyte outgrowth in P4 CGN (cerebellar granule neuron) cells. NT indicates control cells. Y is a compound known to induce neurocyte outgrowth. (* p=,0.05; ** p=,0.01).

FIG. 21 is a photograph of a gel depicting the results from reverse transcriptase polymerase chain reaction (RT PCR) on rat and murine TLR14 (top panel). Control panel with primers without reverse transcriptase is shown in the middle panel. Bottom panel shows the actin RT-PCR control primers as positive controls. The lanes correspond from left-to-right to markers, E19-CN primary cells , P4-CGN cerebellar granule neurons, and primary astrocytes cells. C57B1/6 spleen served as a positive control.

FIGS. 22A and 22B show the nucleotide and amino acid sequence of human NgR1(SEQ ID NO:7 and SEQ ID NO:8), respectively.

FIGS. 23A and 23B show the nucleotide and amino acid sequence of mouse NgR1(SEQ ID NO:9 and SEQ ID NO:10), respectively.

FIGS. 24A and 24B show the nucleotide and amino acid sequence of human p75^{NTR} (SEQ ID NO:11 and SEQ ID NO:12), respectively.

FIGS. 25A and 25B show the nucleotide and amino acid sequence of mouse p75^{NTR} (SEQ ID NO:13 and SEQ ID NO:14), respectively.

FIGS. 26A and 26B show the nucleotide and amino acid sequence of rat NgR (SEQ ID NO:15 and SEQ ID NO:16), respectively.

### DETAILED DESCRIPTION

The present invention is based, at least in part, on the discovery that TLR14 binding agents, e.g., antibodies that bind to the extracellular domain of TLR14 protein, reverse myelin-induced inhibition of neurite outgrowth of cerebellar granule neurons in a dose-dependent manner. Applicants have also shown that TLR14 mRNA and protein are expressed in selected regions of the murine brain, such as hippocampus, substantia nigra, basal ganglia, and cerebral and cerebellar cortex. Elevated TLR14 mRNA expression has been detected in the brains of patients afflicted with Alzheimer's and Parkinson's Disease.

Applicants have also identified that the Leucine Rich Repeat (LRR) region of TLR14 has significant homology (about 37% identity, 47% homology) to the Nogo receptor family of proteins, in particular NgR. The Nogo family of proteins has been shown to regulate neuronal growth. NgR1 is a receptor for three proteins found in myelin, the insulating substance that ensheathes axons (Fournier et al. (2001) Nature 409:341-346, 2001). NgR1 and myelin inhibit nerve growth and prevent nerve regeneration after injury. Co-immunoprecitation studies further revealed that TLR14 interacts with modulators of neuronal growth and/or activity, such as NgR1 and neurotrophin receptor p75^{NTR}. NgR1 and p75^{NTR} have been implicated in modulating neuronal-myelin interactions and signal transduction pathways that ultimately control neurite outgrowth and inhibition (reviewed in Walmsley and Mir (2007) Curr Pharm Des 15:2470-2484, which is incorporated by reference herein). For example, p75^{NTR} expression is increased in response to CNS injury resulting from, e.g., focal ischemia, Alzheimer's disease, seizure and LPS (Hennigan et al. (2007) Brain Research 1130:158-166). p75^{NTR} also increases following more general CNS injury. In addition, NgR ectodomain-Fc has been shown to increase recovery rate following cortical brain injury (J Neuro 2004, 24: 6209-6217). In another example, antibodies to NogoA (NgR1 ligand) have been shown to be protective in MS (Nat Neuroscience (2004) 7: 736-744). In another example, a DNA vaccine designed to generate antibodies to NgR1 protects from spinal cord injury in an animal model (Brain Research (2007) 1147:66-76).

Endotoxins, such as LPS, have been shown to have a negative role on synaptic function presumably via the activation of pro-inflammatory cytokines, such as IL-1β, IL-17 and TNFα. LPS induces TLR14 mRNA and protein expression in the murine brain (Carpenter, S. et al. (April 2007) “A Novel Leucine Rich Repeat Containing Protein Involved in the TLR4 Signaling Pathway,” poster presented at Keystone Symposia entitled “The Macrophage: Homeostasis, Immunoregulation and Disease”). TLR14 also modulates LPS signaling in astrocytes. Id. For example, studies showing that siRNA inhibition of TLR14 reduced LPS-induced signaling events in astrocytoma cell lines confirm a functional role of TLR14 in mediating LPS responses in astrocytic cells. Id. In fact, TLR14 was shown to bind to LPS, and to interact with TLR4, a known mediator of LPS-signaling. Id.

Several publications implicate TLR receptors, such as TLR4 and LPS signaling in mediating responses to neuronal insults and impaired neuronal function. For example, TLR4 expression is increased in microglia and astrocytes following ischemia. In addition, TLR4 deficient mice exhibit reduced cerebral ischemia-reperfusion injury in the MCAO stroke model (Cao et al (2007) BBRC 353: 509-514; Caso et al. (2007) Circulation 115 (12):1599-1608), implicating a role of LPS in stroke. Additional evidence supporting the role of LPS in impaired neuronal differentiation is suggested by the enhanced proliferation and neuronal differentiation in TLR4 deficient mice (Rolls et al (2007) Nat Cell Biol 9 (9):1081-8). LPS has also been shown to induce nitric oxide and MMP-9 in rat primary astrocytes. In addition, LPS injection into the striatum (basal ganglia) reduced neuronal viability by increasing the expression of P2X7R in microglia (J. Neuroscience (2007) 27 (18) 4957-68). These findings confirm Applicants' invention that antagonizing TLR14 function (and/or TLR4 function) may provide neuroprotection of the inflamed and diseased brain.

Lastly, CD14 and TLR4 are constitutively expressed in the choroid plexus and circumventricular organs in the brain. Endotoxemia causes rapid CD14 gene expression with a delayed response in regions surrounding the circumventricular organs (Glezer al at 2007 Neuroscience 147: 867-883). LPS has also been shown to upregulate TLR2 in the brain. TLR2 can be modulated by CD14 on astrocytes to produce proinflammatory cytokines such as CXCL8, IL-6, and IL-12p40 (Bsibsi et al 2007 Glia 55:473-482). Interfering with one or more of these processes by modulating LPS down stream effects would be beneficial at inhibiting neural inflammation.

The findings disclosed herein implicate TLR14 and TLR4 in the regulation of neuronal and glial cell activity and/or growth, and as a mediator of at least some of the endotoxin-induced inflammatory and degenerative events in the brain. TLR14 function may be mediated via an interaction with neuronal modulators, such as NgR1 and/or p75^{NTR}, or by its interaction with TLR4 and LPS signaling. TLR14, therefore, represents an attractive target for therapies designed to improve the regenerative capacity of the CNS. Thus, the present invention provides, in part, methods and compositions for modulating TLR14 function (e.g., neural function) using TLR14 binding agents, e.g., antagonists of TLR14 function. In particular, methods for treating, preventing and/or diagnosing TLR14-associated conditions and/or disorders (e.g., TLR14-associated neurodegenerative conditions and disorders) are disclosed. Screening methods for evaluating TLR14 modulators, e.g., agonists and antagonists of TLR14 function or expression, are also disclosed.

In order that the present invention may be more readily understood, certain terms are first defined. Additional definitions are set forth throughout the detailed description.

The term “Toll-like receptor-14” or “TLR14” refers to a member of the TLR family of receptors that recognize a range of ligands and activate a series of signaling pathways that lead to the induction of immune and inflammatory genes. Members of the TLR family are characterized by a cytosolic domain termed the Toll-IL-IR (TIR) domain and an extracellular region consisting of a series of leucine rich repeats. Occupation of toll-like receptors during infection leads to the expression of a large number of pro-inflammatory proteins, such as inducible cyclooxygenase, adhesion molecules, cytokines, chemokines, and the activation of immune effector cells.

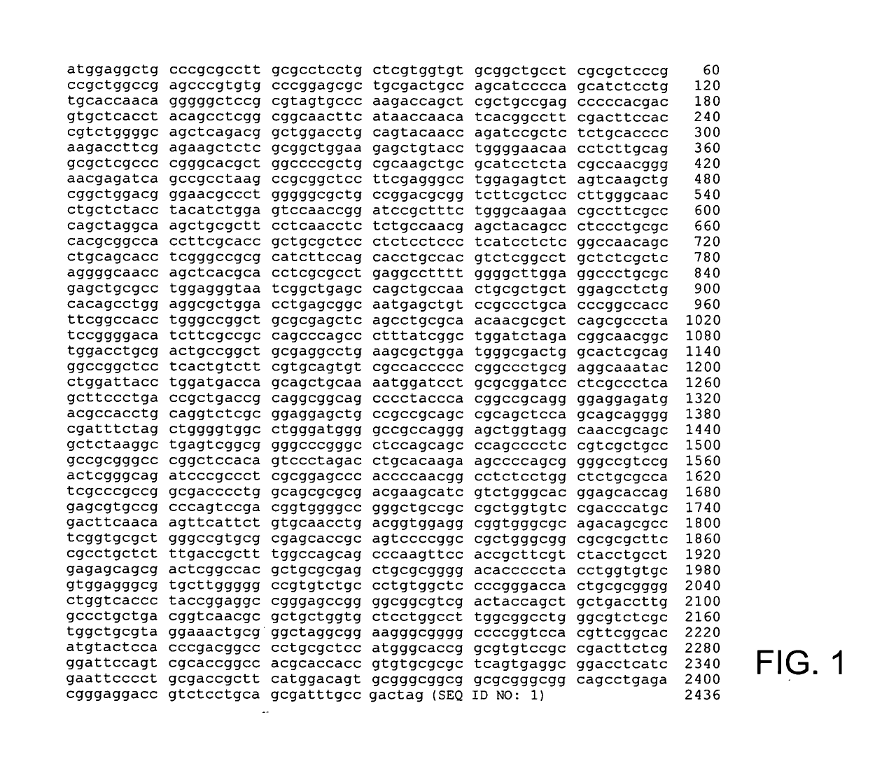

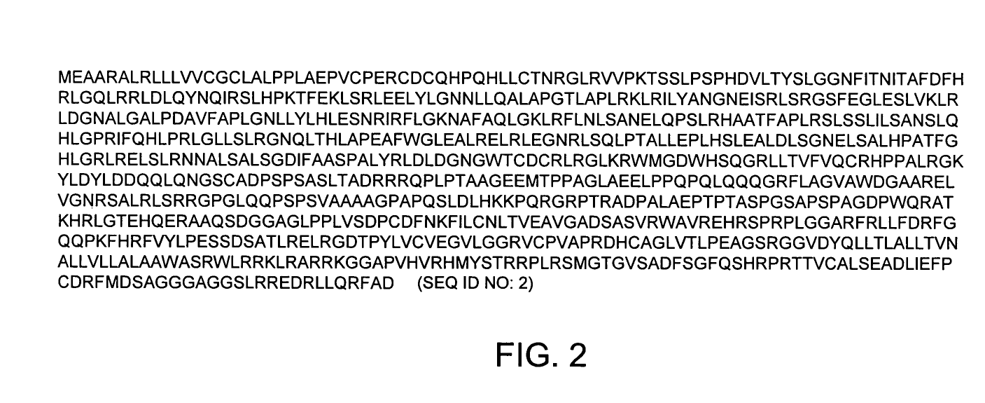

As used herein, “TLR14” refers to a member of the TLR family from any species (e.g., mouse, rat, chicken, dog, bovine, human and non-human primate, but typically of mammalian, e.g., murine, or human or non-human primate origin), as well as any variants thereof (including mutants, fragments, chimerics, and peptidomimetic forms) that retain a TLR14 activity (e.g., TLR14 activity as described herein). Typically, the TLR14 has a biological activity as described herein and one of the following features: (i) an amino acid sequence of a naturally occurring mammalian TLR14 polypeptide or a fragment thereof, e.g., an amino acid sequence shown as FIG. 2 (SEQ ID NO:2) (human) or a fragment thereof (e.g., an extracellular domain of TLR14, e.g., about amino acids 1 to 695 of FIG. 2 (SEQ ID NO:2)); (ii) an amino acid sequence substantially homologous to, e.g., at least 85%, 90%, 95%, 98%, 99% homologous to, an amino acid sequence shown as FIG. 2 (SEQ ID NO:2) (human) or a fragment thereof (e.g., an extracellular domain of TLR14, e.g., about amino acids 1 to 695 of FIG. 2 (SEQ ID NO:2)); (iii) an amino acid sequence which is encoded by a naturally occurring mammalian TLR14 nucleotide sequence or a fragment thereof (e.g., FIG. 1 (SEQ ID NO:1) (human) or a fragment thereof); (iv) an amino acid sequence encoded by a nucleotide sequence which is substantially homologous to, e.g., at least 85%, 90%, 95%, 98%, 99% homologous to, a nucleotide sequence shown as FIG. 1 (SEQ ID NO:1) (human) or a fragment thereof; (v) an amino acid sequence encoded by a nucleotide sequence degenerate to a naturally occurring TLR14 nucleotide sequence or a fragment thereof, e.g., FIG. 1 (SEQ ID NO:1) (human) or a fragment thereof; or (vi) a nucleotide sequence that hybridizes to one of the foregoing nucleotide sequence sequences under stringent conditions, e.g., highly stringent conditions.

The phrase “a biological activity of TLR14 refers to one or more of the biological activities of TLR14, including, but not limited to, (i) modulation of NgR1 binding or activity; (ii) modulation of p75^{NTR} binding or activity; (iii) modulation of LPS signaling, e.g., modulation of one or more endotoxin- (e.g., LPS-) mediated inflammatory signals, in the brain (e.g., modulation of one or more of: LPS binding to TLR14, IκB degradation, phosphorylation of p38, pro-inflammatory cytokine or chemokine secretion, induction of nitric oxide production in, e.g., astrocytes, or modulation of neuronal viability and/or gliosis; and/or (iv) modulation of one or more of: neuronal cell survival, differentiation and/or neurite outgrowth.

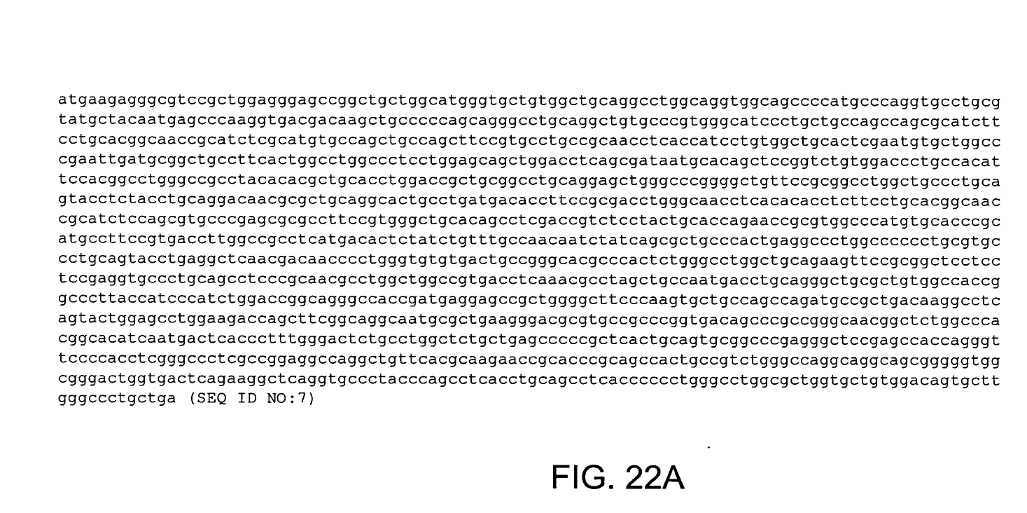

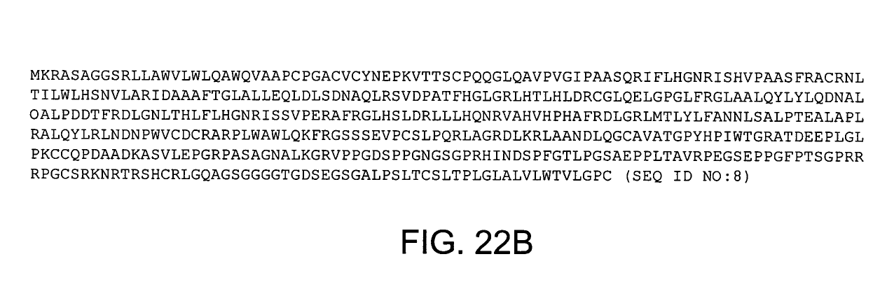

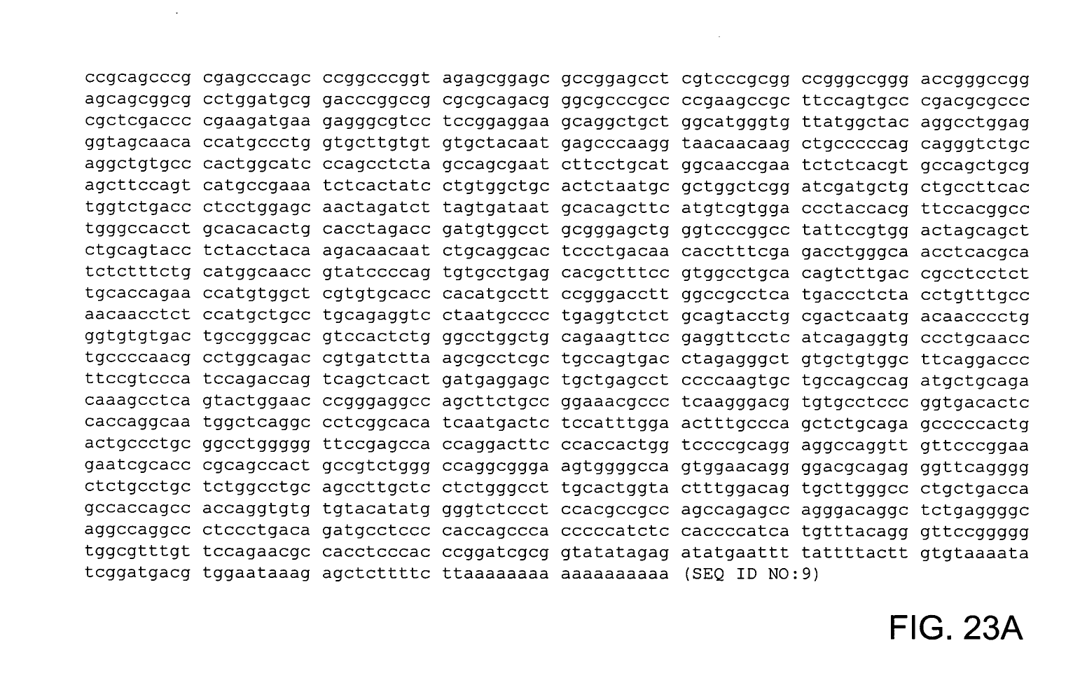

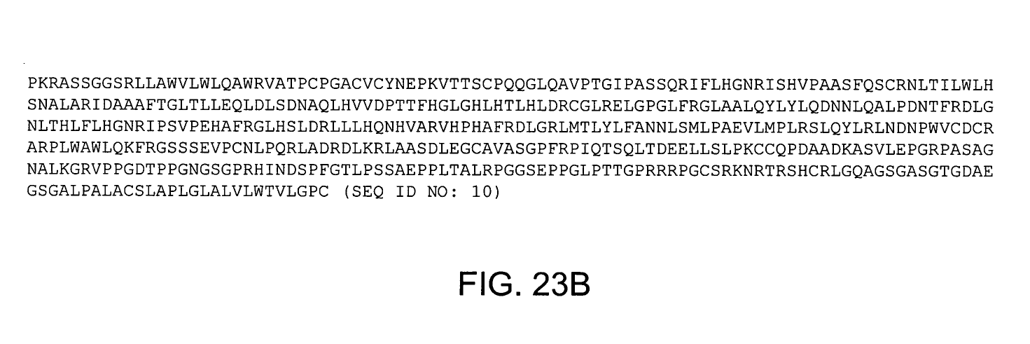

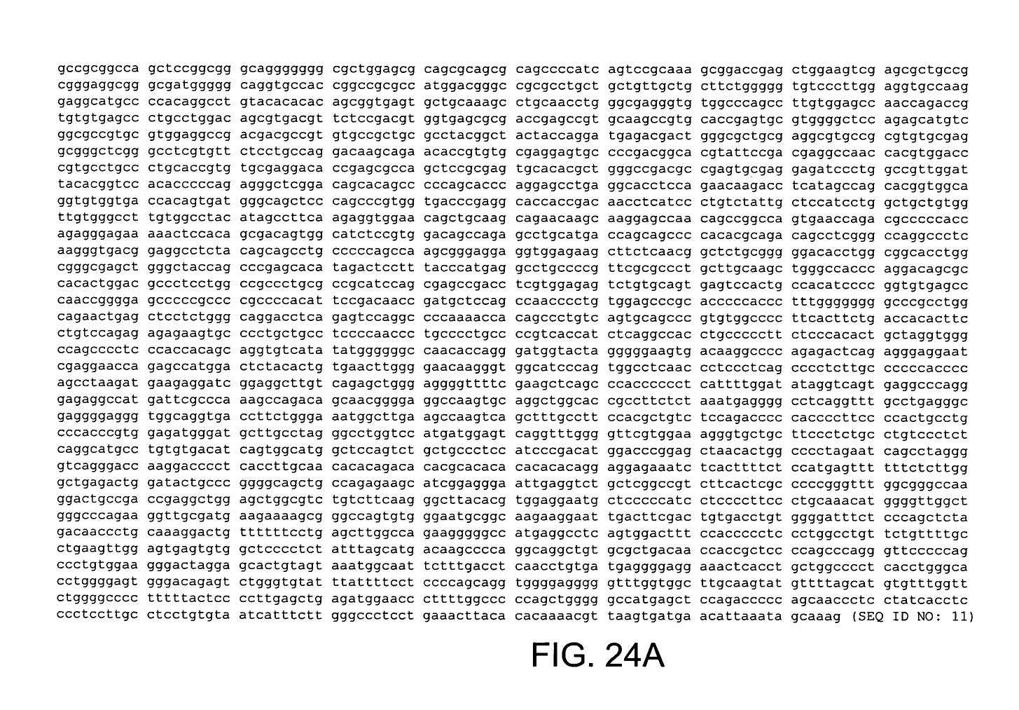

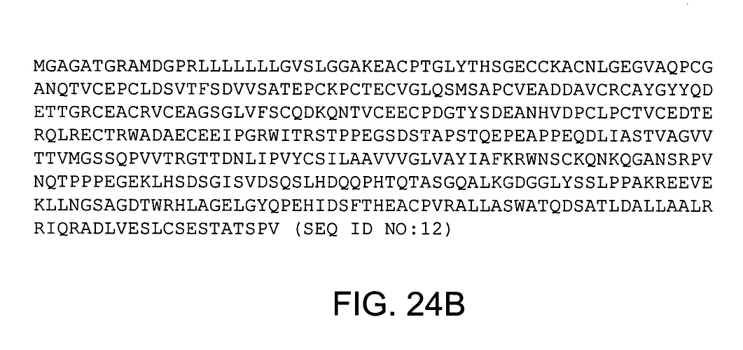

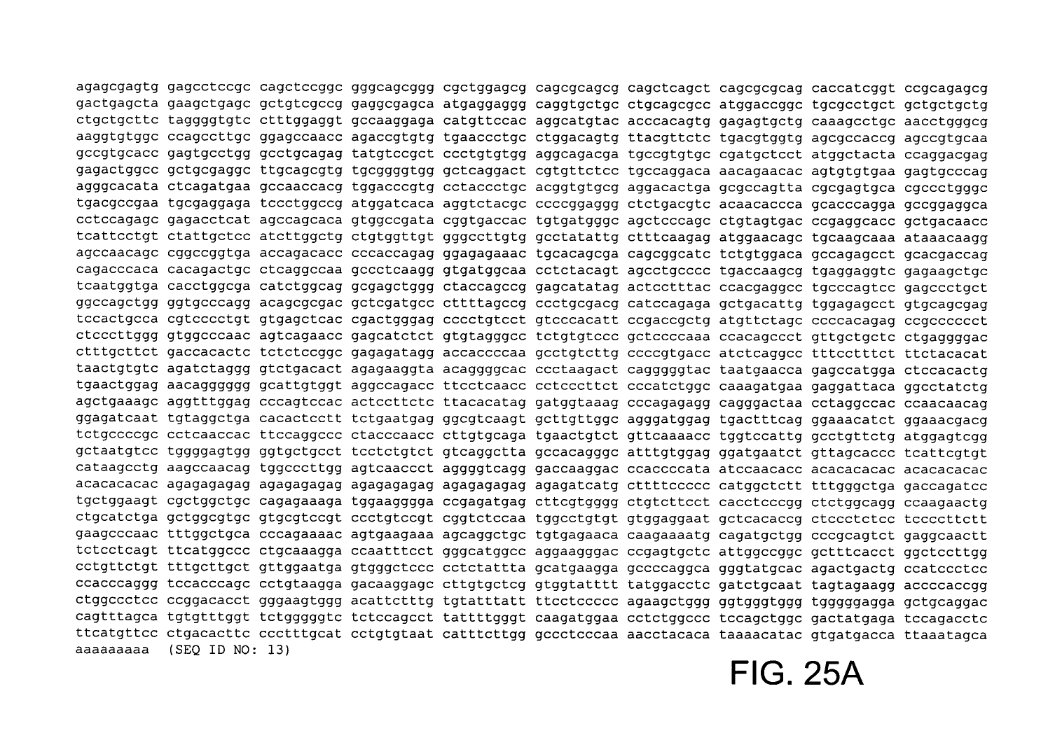

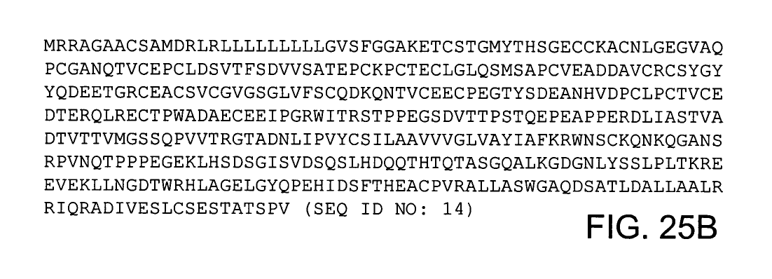

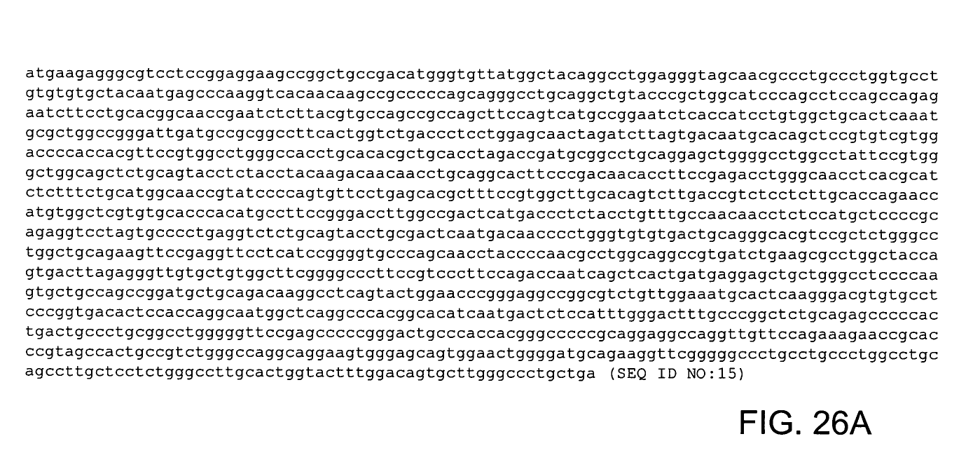

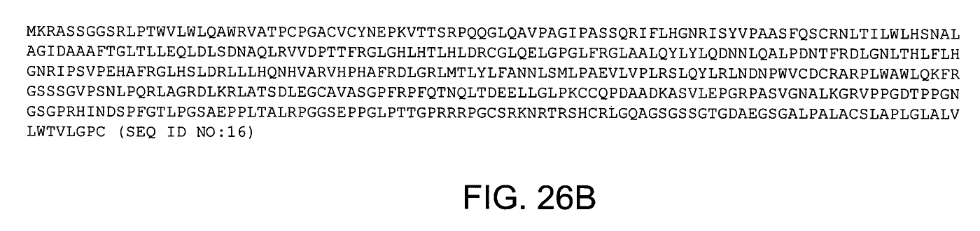

As used herein, the term “TLR1-associated” protein or polypeptide refers to a protein or polypeptide from any species that is associated physically and/or functionally to TLR14. For example, this term includes NgR1 or p75^{NTR}, which have been shown herein to interact with TLR14. The nucleotide and amino acid sequence of human and mouse NgR1 are depicted herein as FIGS. 22A-22B (SEQ ID NO:7 and SEQ ID NO:8) (human), and as FIGS. 23A-23B (SEQ ID NO:9 and SEQ ID NO:10) (mouse); and FIGS. 24A-24B (SEQ ID NO:11 and SEQ ID NO:12) (human) and as FIGS. 25A-25B (SEQ ID NO:13 and SEQ ID NO:14) (see also, Barton et al., EMBO., 22:3291-3302, 2003; Lauren et al., Neurosci., 24:581-594, 2003; Ai et al., Development, 1343:3327-3338, 2007; and McGregor et al., Am. J. Med. Genet. B. Neuropsychiatr. Genet., 144:696-700, 2007, for a description and characterization of NgR1 and p75^{NTR}).

The methods and compositions of the present invention encompass TLR14 and TLR14-associated polypeptides, e.g., NgR1 or p75^{NTR}, and nucleic acids having the sequences specified, or sequences substantially identical or similar thereto, e.g., sequences at least 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%. 91%, 92%, 93%, 94%, 95%, 96%, 97%, 98% or 99% identity to SEQ ID NO:2, SEQ ID NO:4, SEQ ID NO:8, or SEQ ID NO:12 are termed substantially identical.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%. 91%, 92%, 93%, 94%, 95%, 96%, 97%, 98% or 99% identity to SEQ ID NO:1 and 3 are termed substantially identical.

The term “functional variant” refers to polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In some embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two amino acid sequences is determined using the Needleman and Wunsch ((1970) J. Mol. Biol. 48:444-453) algorithm which has been incorporated into the GAP program in the GCG software package (available at gcg.com), using either a Blossum 62 matrix or a PAM250 matrix, and a gap weight of 16, 14, 12, 10, 8, 6, or 4 and a length weight of 1, 2, 3, 4, 5, or 6. In some embodiments, the percent identity between two nucleotide sequences is determined using the GAP program in the GCG software package (available at gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and)(BLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to TLR14/TLR14-associated protein nucleic acid (SEQ ID NO:1) molecules of the invention. BLAST protein searches can be performed with the XBLAST program, score=50, wordlength=3 to obtain amino acid sequences homologous to TLR14/TLR14-associated protein (SEQ ID NO:1) protein molecules of the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See ncbi.nlm.nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C.

It is understood that the TLR14 and TLR14 modulators, e.g., TLR14 binding agents (e.g., TLR14 antagonists) of the present invention may have additional conservative or non-essential amino acid substitutions, which do not have a substantial effect on their functions.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Various aspects of the invention are described in further detail below.

### Methods of Treatment

The invention provides methods for modulating, e.g., antagonizing TLR14 function (e.g., by reducing, attenuating, or otherwise decreasing the amplitude and intensity of one or more biological activities of TLR14 in, for example, a neural cell and/or tissue. The method includes contacting the cell and/or tissue with a TLR14 modulator, e.g., a TLR14 binding agent that antagonizes human TLR14, in an amount sufficient to modulate (e.g., decrease), the function of the TLR14-responsive cell and/or tissue (or the biological activity of TLR14 in the cell or tissue). The term “TLR14-responsive cell and/or tissue” refers to any cell and/or tissue capable of transducing a signal, e.g., IκB degradation or p38 phosphorylation, in response to TLR14.

As used herein, a “TLR14 antagonist” that is useful in the method of the invention, refers to an agent which reduces, inhibits or otherwise diminishes one or biological activities of TLR14 or of a TLR14-associated polypeptide. The antagonist can interact with, e.g., binds to, a TLR14/TLR14-associated polypeptide. Antagonism using a TLR14/TLR14-associated polypeptide antagonist does not necessarily indicate a total elimination of the TLR14/TLR14-associated polypeptide biological activity. For example, the TLR14 antagonist interacts with, e.g., binds to, and/or the ligand binding site of TLR14, such that activation and/or downstream signaling of TLR14 is reduced or inhibited. In certain embodiments, the TLR14 antagonist interferes with LPS binding to TLR14 and/or activation of LPS-induced signaling (e.g., LPS-induced IkB degradation and phosphorylation of p38).

In some embodiments, the TLR14 responsive cell or tissue is, e.g., a neuronal cell (e.g., a dopaminergic, cholinergic, cortical, cerebellar, hippocampal, spinal cord neuronal cell) and/or a glial cell (e.g., microglia, astrocytes and oligodendrocytes). As herein defined, the cells of the central nervous system (CNS) include, but are not limited to; neuronal cells and human glial cells. The particular human glial cells can be chosen from one or more of microglia, astrocytes and oligodendrocytes.

Exemplary TLR14 activities that can be modulated (e.g., attenuated) using the methods and compositions of the present invention include one or more of: (i) modulation of NgR1 binding or activity; (ii) modulation of p75^{NTR} binding or activity; (iii) modulation of LPS signaling, e.g., modulation of one or more endotoxin—(e.g., LPS-) mediated inflammatory signals, in the brain (e.g., modulation of one or more of endotoxin binding to TLR14, IκB degradation, phosphorylation of p38, pro-inflammatory cytokine or chemokine secretion, induction of nitric oxide production and MMP-9 expression in, e.g., astrocytes, or modulation of neuronal viability and/or gliosis; and/or (iv) modulation of one or more of: neuronal cell survival, differentiation and/or neurite outgrowth.

The methods of the invention can be performed on cells (e.g., neural cells and/or tissue) present in a subject, e.g., as part of an in vivo (e.g., therapeutic or prophylactic) protocol, or in an animal subject (e.g., an in vivo animal model, such as a middle cerebral artery occlusion (MCAO) or stroke model, a CNS inflammatory model (e.g., EAE, lymphocytic meningeal encephalitis models, Venezuelan equine encephalitis infection, meningeal encephalitis, Pneumococous CNS inflammation, or a genetically modified model, e.g., an animal model having overexpressed TLR14 or a mutation in a TLR receptor (e.g., a TLR14 deficient animal). For in vivo methods, the TLR14 antagonist, alone or in combination with another agent, can be administered to a subject, e.g., a mammal, suffering from a neurodegenerative condition and/or disorder, or suffering from the symptoms of a neurodegenerative condition and/or disorder, in an amount sufficient to antagonize TLR14 function, or one or more TLR14 activities in the subject, and/or diminish or alleviate one or more symptoms of a neurodegenerative condition or disorder. The neurodegenerative condition to be treated may be an acute neurodegenerative condition, or it may be a chronic or progressive condition.

TLR14 modulations can also be used as a regenerative therapy for neurological diseases and disorders. This extends to pharmaceutical compositions and methods for the regeneration or repair of neural tissue. Although axonal regeneration in damaged peripheral nerves does occur, axonal regeneration in the CNS, for example, is minimal, and as such, neural damage or trauma resulting from disease or injury to the brain or spinal cord can be severe and prolonged. Molecules which inhibit axonal re-growth have been identified by the inventors and TLR14 modulators, e.g., TLR14 binding agents, have surprisingly identified that TLR14 modulators (e.g., TLR14 binding agents) that have utility in inducing axonal regeneration have been identified herein.

Accordingly in a further embodiment, the invention provides a method of regenerating neurons. The method comprises administering to a subject in need thereof a TLR14 modulator (e.g., a TLR14 binding agent as described herein) in an amount effective to induce neuronal regeneration.

As herein defined, the term ‘neuronal regeneration’ relates to neurite outgrowth. In particular, this term comprises inhibition of the collapse of neuron growth cones and/or a decrease in the inhibition of neurite outgrowth and sprouting in a neuron. The outgrowth and sprouting can be axonal growth, and the neuron can be a CNS neuron.

In practicing the method of treatment or use of the present invention, a therapeutically effective amount of a TLR14 modulator, e.g., TLR14 binding agent (e.g., TLR14 antagonist) may be administered either alone or in combination with other therapies such as treatments.

The subject to whom the TLR14 modulator, e.g., the TLR14 binding agent, e.g., antagonist, is administered can be a mammal, e.g., a human, suffering from, for example, a neurodegenerative condition and/or disorder characterized by aberrant TLR14 activity. In certain embodiments, the neurodegenerative disorder or condition is an acute neurodegenerative condition, or a chronic or progressive neurodegenerative condition. As herein defined, the term “neurodegenerative disorder” (or condition) is an inclusive term encompassing acute and chronic conditions, disorders or diseases of the central or peripheral nervous system. A neurodegenerative condition may be age-related, or it may result from injury or trauma, or it may be related to a specific disease or disorder.

Acute neurodegenerative conditions include, but are not limited to, conditions associated with neuronal cell death or compromise including cerebrovascular insufficiency, focal or diffuse brain trauma, diffuse brain damage, spinal cord injury or peripheral nerve trauma, e.g., resulting from physical or chemical burns, deep cuts or limb severance. Examples of acute neurodegenerative disorders are: cerebral ischemia or infarction including embolic occlusion and thrombotic occlusion, reperfusion following acute ischemia, perinatal hypoxic-ischemic injury, cardiac arrest, intracranial and intravertebral lesions (such as contusion, penetration, shear, compression and laceration). Chronic neurodegenerative conditions include, but are not limited to, Alzheimer's disease, Pick's disease, diffuse Lewy body disease, progressive supranuclear palsy (Steel-Richardson syndrome), multisystem degeneration (Shy-Drager syndrome), chronic epileptic conditions associated with neurodegeneration, motor neuron diseases including amyotrophic lateral sclerosis, degenerative ataxias, cortical basal degeneration, ALS-Parkinson's-Dementia complex of Guam, subacute sclerosing panencephalitis, Huntington's disease, Parkinson's disease, synucleinopathies (including multiple system atrophy), primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, familial dysautonomia (Riley-Day syndrome), and prion diseases (including, but not limited to Creutzfeldt-Jakob disesase (CJD), Gerstmann-Sträussler-Scheinker disease, Kuru and fatal familial insomnia), demyelination diseases and disorders including multiple sclerosis and hereditary diseases such as leukodystrophies.

Other neurodegenerative conditions include various neuropathies, such as multifocal neuropathies, sensory neuropathies, motor neuropathies, sensory-motor neuropathies, infection-related neuropathies, autonomic neuropathies, sensory-autonomic neuropathies, demyelinating neuropathies (including, but not limited to, Guillain-Barre syndrome and chronic inflammatory demyelinating polyradiculoneuropathy), other inflammatory and immune neuropathies, neuropathies induced by drugs, neuropathies induced by pharmacological treatments, neuropathies induced by toxins, traumatic neuropathies (including, but not limited to, compression, crush, laceration and segmentation neuropathies), metabolic neuropathies, endocrine and paraneoplastic neuropathies, among others.

Further neurodegenerative conditions include dementias, regardless of underlying etiology, including age-related dementia and other dementias and conditions with memory loss including dementia associated with Alzheimer's disease, vascular dementia, diffuse white matter disease (Binswanger's disease), dementia of endocrine or metabolic origin, dementia of head trauma and diffuse brain damage, dementia pugilistica and frontal lobe dementia.

Exemplary particular disorders include, but are not limited to, CNS disorders characterized by CNS injury and/or axonal degeneration, Alzheimer's disease, Parkinson's disease, olivopontocerebellar atrophy, multiple sclerosis (MS), Pick's disease, mild cognitive impairment (MCI), amyotrophic lateral sclerosis (ALS), Huntington's disease, prion diseases such as CJD, AIDS-related dementia, encephalitis, stroke, traumatic brain injury; spinal cord injury, multiple sclerosis, neuropathy associated with medical treatments such as chemotherapy, and ischemia or ischemia-induced injury. The neurodegenerative condition or disorder can also include infections and neuroinflammatory conditions, such as CNS conditions caused /by bacterial infections (e.g., Staphilococous, Streptococous and pneumococous) and encephalitis.

As used herein, the term “therapeutically effective amount” means the total amount of each active component of the pharmaceutical composition or method that is sufficient to show a meaningful patient benefit, e.g., amelioration of symptoms of, healing of, or increase in rate of healing of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

When co-administered with one or more agents, a TLR14 antagonist and/or a TLR14-associated polypeptide -antagonist may be administered either simultaneously with the second agent, or sequentially. If administered sequentially, the attending physician will decide on the appropriate sequence of administering a TLR14 antagonist/TLR14-associated polypeptide-antagonist in combination with other agents. The term “in combination” in this context means that the agents are given substantially contemporaneously, either simultaneously or sequentially. If given sequentially, at the onset of administration of the second compound, the first of the two compounds is preferably still detectable at effective concentrations at the site of treatment.

Therapies that can be used in combination with TLR14 modulators to treat Parkinson's Disease include, but are not limited to, one or more of the following: antioxidants (e.g., alpha-tocopherol); anticholinergic agents; dopamine precursor (e.g., levodopa/carbidopa); direct acting dopamine agonists (e.g., ergot derivatives, for example, bromocriptine and pergolide; and non-ergolines, for example, pramipexole and ropinirole); indirect-acting dopamine agonists (e.g., selegiline and amantadine); or catechol-O-methyltransferase (COMT) inhibitors (e.g., tolcapone and entacapone).

Therapies that can be used in combination with TLR14 modulators to treat Alzheimer's Disease include, but are not limited to, one or more of the following: antioxidants (e.g., alpha-tocopherol); cholinesterase inhibitors (e.g., tacrine, donepezil, rivastigmine, galantamine, and metrifonate); N-Methyl-D-Aspartate (NMDA) antagnosists (e.g., memantine, amantadine, rimantadine, and ketamine); anti-inflammatory agents (e.g., propentifyline and selective COX2 inhibitors, for example, celecoxib and rofecoxib); chelating agents (e.g., cliquinol); oestrogens (e.g., selective oestrogen receptor modulators); or secretase inhibitors.

Therapies that can be used in combination with TLR14 modulators to treat spinal cord injuries include, but are not limited to, one or more of the following: methylprednisolone sodium succinate; antioxidants; membrane stabilizers; glutamate antagonists; anti-inflammatories; caspase inhibitors; or calpain inhibitors.

The TLR14 modulator, e.g., antagonists, and TLR14-associated polypeptide antagonists (also referred to herein as “active compounds”) of the invention can be incorporated into pharmaceutical compositions. Such compositions typically include the nucleic acid molecule, protein, or antibody and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

When a therapeutically effective amount of a TLR14 modulator, e.g., antagonist, TLR14-associated polypeptide antagonist is administered orally, the binding agent will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% binding agent, and preferably from about 25 to 90% binding agent. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of the binding agent, and preferably from about 1 to 50% the binding agent.

When a therapeutically effective amount of a TLR14 modulator, e.g., antagonists or a TLR14-associated polypeptide- antagonist is administered by intravenous, cutaneous or subcutaneous injection, binding agent will be in the form of a pyrogen-free, parenterally acceptable aqueous solution. The preparation of such parenterally acceptable protein solutions, having due regard to pH, isotonicity, stability, and the like, is within the skill in the art. A preferred pharmaceutical composition for intravenous, cutaneous, or subcutaneous injection should contain, in addition to binding agent an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additive known to those of skill in the art.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The TLR14 modulator may be delivered to the brain or CNS by means of an intracerebroventricular injection (icy / ICV), or through delivery by a minipump, or by using a viral vector targeted to the brain or neural tissue, or by directly administering naked plasmid DNA encoding for the modulator. Alternatively any other suitable delivery mechanism may be employed which results in the modulator being delivered to the brain or CNS. Examples of further suitable routes of delivery are provided hereinafter.

In particular embodiments, the TLR14 modulator is delivered to glia cells, in particular microglia, but also astrocytes and/or oligodendrocytes. In certain embodiments, the modulator is administered with at least one other cell type, such as an astrocyte, oligodendrocyte, neuron, neural progenitor, neural stem cell or other multipotent or pluripotent stem cell. In these embodiments, the other cell type can be administered simultaneously with, before or after the modulator. Likewise, in these or other embodiments, the TLR14 modulator is administered with at least one other agent, such as a drug for neural therapy, or another beneficial adjunctive agent such as an anti-inflammatory agent, anti-apoptotic agents, antioxidant or growth factor. In these embodiments, the other agent can be administered simultaneously with, before or after the modulator.

In further embodiments, the cells are administered at a pre-determined site in the central or peripheral nervous system of the subject, e.g., a patient. They can be administered by injection or infusion, or encapsulated within an implantable device, or by implantation of a matrix or scaffold containing the cells, or by any other means.

In some embodiments, the active compounds will be administered directly to the brain or another suitable site of the central nervous system (CNS). Using these methods, the TLR14 antagonists will be delivered directly to the hippocampus, for example, to microglial cells and neuronal cells. In some embodiments, the TLR14 antagonist may be administered by means of intracerebroventricular injection (ICV), intrathecal administration (e.g., using an intrathecal drug delivery pump). In some embodiments, the active compounds may be administered by means of a catheter and pump system, such as a fully implantable pump system or an external pump system.

Suitable pump and catheter systems are commercially available, e.g., SynchroMed® pump and InDura® intrathecal catheters (both from Medtronic Sofamor Danek, Memphis, Tenn.).

Alternatively a viral vector may be used to target delivery of the TLR14 modulator to the brain, CNS or neural tissue. Such a vector will include a construct which contains a gene encoding for the modulator in cases where the modulator is a protein, such as an antibody, in particular a monoclonal antibody or other similar binding fragment. The construct may further contain a promoter which is provided adjacent to the gene and which controls expression of the gene. Viral vectors which may be suitable for such delivery and targeting may be nonreplicative herpes simplex type 1 viruses (see for example Poliani et al. Hum Gene Ther. 2001 20;12(8):905-20.); Semliki Forest virus, (see Jerusalmi et al. Mol. Ther. 2003 8(6):886-94.) and adenovirus, (for example, see Braciack et al. J. Immunol. 2003 Jan. 15; 170(2):765-74.)

Different neurological conditions, such as neurodegenerative diseases affect the brain in different ways and also affect different areas of the brain tissue. Accordingly, in certain further aspects, the compositions of the invention may be administered to distinct areas of the brain directly in order to optimize the therapeutic effect.

In certain embodiments, the compound which inhibits the expression or biological function of Toll-like Receptor 14 is administered to the hippocampus, and in particular to microglial cells.

In some embodiments, the TLR14 modulator are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In those embodiments where the modulator is a biologic, e.g., an antibody molecule, about 1 μg/kg to 15 mg/kg of antagonist is an initial candidate dosage for administration to the patient depending on the type and severity of the disease, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is repeated until a desired suppression of disease symptoms occurs. However, other dosage regimens may be useful. The progress of this therapy is easily monitored by conventional techniques and assays, including, for example, radiographic tumor imaging.

The duration of therapy using the pharmaceutical composition of the present invention will vary, depending on the severity of the disease being treated and the condition and potential idiosyncratic response of each individual patient.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, preferably between 2 to 8 weeks, more preferably between about 3 to 7 weeks, and even more preferably for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, a series of treatments.

For antibodies, the preferred dosage is 0.1 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

For small molecules, exemplary doses include milligram or microgram amounts of the small molecule per kilogram of subject or sample weight, e.g., about 1 microgram per kilogram to about 500 milligrams per kilogram, about 100 micrograms per kilogram to about 5 milligrams per kilogram, or about 1 microgram per kilogram to about 50 micrograms per kilogram. It is further understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, half-life, any drug combination, and the degree of expression or activity to be modulated.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

### TLR14 Modulators

### Antibody Molecules

In some embodiments, the TLR14 modulators, e.g., binding agents, are antibody molecules against TLR14. In some embodiments, the antibody molecule is a monoclonal or single specificity antibody, or an antigen-binding fragment thereof (e.g., an Fab, F(ab′)₂, Fv, a single chain Fv fragment, a shark variant or a camelid variant) that binds to TLR14, e.g., a mammalian (e.g., human, TLR14 (or a functional variant thereof)).

Typically, the antibody molecule is a human, humanized, chimeric, camelid, or an in vitro generated antibody to human TLR14 (or functional fragment thereof). Typically, the antibody inhibits, reduces or neutralizes one or more activities of TLR14 (e.g., one or more biological activities of TLR14 as described herein). In some embodiments, the antibody molecule binds to TLR14 (e.g., about amino acids 1-811 of FIG. 2 (SEQ ID NO: 2), or an epitope comprising fragments thereof, or a sequence substantially homologous thereto). For example, the antibody molecule can bind to the extracellular domain of TLR14 (e.g., about amino acids 1 to 695 or 696, or a fragment thereof, e.g., about amino acids 85 to 103, or 509 to 526, of FIG. 2 (SEQ ID NO:2). In other embodiments, the antibody molecule binds to a Leucine-rich domain of TLR14, e.g., a domain located at about amino acids 26 to 58, 82 to 105, 106 to 129, 130 to 153, 154 to 177, 178 to 201, 202 to 227, 228 to 251, 252 to 275, 276 to 299, 300 to 323, 324 to 347, or 359 to 315, of FIG. 2 (SEQ ID NO:2). In yet another embodiment, the antibody molecule binds to a fibronectin-type III domain (FN3) located at about amino acids 589 to 671, of FIG. 2 (SEQ ID NO:2), and/or the RGD integrin binding site located at about amino acids 640 to 660, 645 to 655, or 652 to 654, of FIG. 2 (SEQ ID NO:2). Human TLR14 protein may include a leader sequence about 20-21 amino acids long corresponding to about amino acids 1 to 20 of SEQ ID NO:2. In yet other embodiments, the antibody molecule binds to an intracellular domain of TLR14, e.g., at about amino acids 720 to 811, or a portion thereof (e.g., about amino acids 796 to 811 of FIG. 2 (SEQ ID NO:2)). In yet other embodiments, the antibody molecule competes for binding to TLR14 with any of antibodies 0540 (A), 0541 (B), and 0547 (C) describes herein (e.g., the antibody molecule binds to an epitope on TLR14 that is the same or substantially similar to the one recognized by antibodies 0540 (A), 0541 (B), and 0547 (C). In other embodiments, the antibody molecule binds to an epitope as described herein and inhibits, reduces or neutralizes one or more activities of TLR14.

In certain embodiments, the antibody molecule binds to a TLR14-interacting polypeptide, e.g., NgR, e.g., a human NgR; or p75^{NTR}, e.g., a human p75^{NTR} (e.g., an NgR, or p75^{NTR} comprising an amino acid sequence identical to a mammalian, e.g., human, NgR, or p75^{NTR} as shown in FIG. 22B (SEQ ID NO:8) or FIG. 24B (SEQ ID NO:12), respectively), or a sequence substantially homologous thereto, and inhibits, reduces or neutralizes one or more activities of TLR14.

As used herein, the term “antibody molecule” refers to a protein comprising at least one immunoglobulin variable domain sequence. The term antibody molecule includes, for example, full-length, mature antibodies and antigen-binding fragments of an antibody. For example, an antibody molecule can include a heavy (H) chain variable domain sequence (abbreviated herein as VH), and a light (L) chain variable domain sequence (abbreviated herein as VL). In another example, an antibody molecule includes two heavy (H) chain variable domain sequences and two light (L) chain variable domain sequence, thereby forming two antigen binding sites. Examples of antigen-binding fragments include: (i) a Fab fragment, a monovalent fragment consisting of the VL, VH, CL and CH1 domains; (ii) a F(ab')2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment, which consists of a VH domain; (vi) a camelid or camelized variable domain; and (vii) a single chain Fv (scFv), see e.g., Bird et al. (1988) Science 242:423-426; and Huston et al. (1988) Proc. Natl. Acad. Sci. USA 85:5879-5883). These antibody fragments are obtained using conventional techniques known to those with skill in the art, and the fragments are screened for utility in the same manner as are intact antibodies.

The VH and VL regions can be subdivided into regions of hypervariability, termed “complementarity determining regions” (CDR), interspersed with regions that are more conserved, termed “framework regions” (FR). The extent of the framework region and CDRs has been precisely defined by a number of methods (see, Kabat, E. A., et al. (1991) Sequences of Proteins of Immunological Interest, Fifth Edition, U.S.

Department of Health and Human Services, NIH Publication No. 91-3242; Chothia, C. et al. (1987) J. Mol. Biol. 196:901-917; and the AbM definition used by Oxford Molecular's AbM antibody modelling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). Generally, unless specifically indicated, the following definitions are used: AbM definition of CDR1 of the heavy chain variable domain and Kabat definitions for the other CDRs. In addition, embodiments of the invention described with respect to Kabat or AbM CDRs may also be implemented using Chothia hypervariable loops. Each VH and VL typically includes three CDRs and four FRs, arranged from amino-terminus to carboxy-terminus in the following order: FR1, CDR1, FR2, CDR2, FR3, CDR3, FR4.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding site” refers to the part of an antibody molecule that comprises determinants that form an interface that binds to TLR14, or an epitope thereof. With respect to proteins (or protein mimetics), the antigen-binding site typically includes one or more loops (of at least four amino acids or amino acid mimics) that form an interface that binds to TLR14. Typically, the antigen-binding site of an antibody molecule includes at least one or two CDRs, or more typically at least three, four, five or six CDRs.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

The anti- TLR14 antibody can be a polyclonal or a monoclonal antibody. In other embodiments, the antibody can be recombinantly produced, e.g., produced by phage display or by combinatorial methods.

Phage display and combinatorial methods for generating anti-TLR14 antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibody Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the TLR14 antibody is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), or camel antibody. Preferably, the non-human antibody is a rodent (mouse or rat antibody). Method of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur Jlmmunol 21:1323-1326).

An anti-TLR14 antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Patent No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immuoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to a TLR14/TLR14-associated protein or a fragment thereof. Preferably, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). The acceptor framework is a naturally-occurring (e.g., a human) framework or a consensus framework, or a sequence about 85% or higher, preferably 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art. Humanized antibodies can be generated by replacing sequences of the Fv variable region which are not directly involved in antigen binding with equivalent sequences from human Fv variable regions. General methods for generating humanized antibodies are provided by Morrison, S. L., 1985, Science 229:1202-1207, by Oi et al., 1986, BioTechniques 4:214, and by Queen et al. U.S. Pat. No. 5,585,089, U.S. Pat. No. 5,693,761 and U.S. Pat. No. 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acid are known to those skilled in the art and, for example, may be obtained from a hybridoma producing an antibody against a TLR14 polypeptide or fragment thereof. The recombinant DNA encoding the humanized antibody, or fragment thereof, can then be cloned into an appropriate expression vector.

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare the humanized antibodies of the present invention (UK Patent Application GB 2188638A, filed on Mar. 26, 1987; Winter U.S. Pat. No. 5,225,539), the contents of which is expressly incorporated by reference.

Also within the scope of the invention are humanized antibodies in which specific amino acids have been substituted, deleted or added. Preferred humanized antibodies have amino acid substitutions in the framework region, such as to improve binding to the antigen. For example, a humanized antibody will have framework residues identical to the donor framework residue or to another amino acid other than the recipient framework residue. To generate such antibodies, a selected, small number of acceptor framework residues of the humanized immunoglobulin chain can be replaced by the corresponding donor amino acids. Preferred locations of the substitutions include amino acid residues adjacent to the CDR, or which are capable of interacting with a CDR (see e.g., U.S. Pat. No. 5,585,089). Criteria for selecting amino acids from the donor are described in U.S. Pat. No. 5,585,089, e.g., columns 12-16 of U.S. Pat. No. 5,585,089, the e.g., columns 12-16 of U.S. Pat. No. 5,585,089, the contents of which are hereby incorporated by reference. Other techniques for humanizing antibodies are described in Padlan et al. EP 519596 Al, published on Dec. 23, 1992.

In one embodiment, an antibody can be made by immunizing with purified TLR14/TLR14 associated-antigen, or a fragment thereof, e.g., a fragment described herein, membrane associated antigen, tissue, e.g., crude tissue preparations, whole cells, preferably living cells, lysed cells, or cell fractions, e.g., membrane fractions.

The anti-TLR14 antibody can be a single chain antibody, or a fragment thereof. A single-chain antibody (scFV) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target TLR14 protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has effector function and can fix complement. In other embodiments the antibody does not recruit effector cells or fix complement. In another embodiment, the antibody has reduced or no ability to bind an Fc receptor. For example., it is a isotype or subtype, fragment or other mutant, which does not support binding to an Fc receptor, e.g., it has a mutagenized or deleted Fc receptor binding region.

An anti-TLR14 antibody (e.g., monoclonal antibody) can be used to isolate TLR14 by standard techniques, such as affinity chromatography or immunoprecipitation. Moreover, an TLR14 antibody can be used to detect TLR14 protein (e.g., in a cellular lysate or cell supernatant) in order to evaluate the abundance and pattern of expression of the protein. Anti-TLR14 antibodies can be used diagnostically to monitor protein levels in tissue as part of a clinical testing procedure, e.g., to determine the efficacy of a given treatment regimen. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H. The invention also includes a nucleic acid which encodes an TLR14 antibody, e.g., an anti- TLR14 antibody described herein. Also included are vectors which include the nucleic acid and cells transformed with the nucleic acid, particularly cells which are useful for producing an antibody, e.g., mammalian cells, e.g. CHO or lymphatic cells.

The invention also includes cell lines, e.g., hybridomas, which make an anti-TLR14 antibody, e.g., and antibody described herein, and method of using said cells to make a TLR14 antibody.

Also featured are nucleic acids encoding the TLR14 sequence and variants thereof. The polypeptide can be used to provide a TLR14 binding agent that binds TLR14, and optionally, also a TLR14 from another species.

In one aspect, the invention features a method of providing a target binding molecule that specifically binds to TLR14. For example, the target binding molecule is an antibody molecule. The method includes: providing a target protein that comprises at least a portion of non-human protein, the portion being homologous to (at least 70, 75, 80, 85, 87, 90, 92, 94, 95, 96, 97, 98% identical to) a corresponding portion of a human target protein, but differing by at least one amino acid (e.g., at least one, two, three, four, five, six, seven, eight, or nine amino acids); obtaining a binding agent that specifically binds to the antigen; and evaluating efficacy of the binding agent in modulating activity of the target protein. The method can further include administering the binding agent (e.g., antibody molecule) or a derivative (e.g., a humanized antibody molecule) to a human subject. In some embodiments, the target protein is TLR14.

In one embodiment, the step of obtaining comprises using a protein expression library, as described above. For example, the library displays antibody molecules such as Fab's of scFv's. In one embodiment, the step of obtaining comprises immunizing an animal using the antigen as an immungen. For example, the animal can be a rodent, e.g., a mouse or rat. The animal can be a transgenic animal.

### TLR14 Peptides

In some embodiments, the TLR14 antagonist is a TLR14 antagonistic pro-peptide (e.g., a truncated or variant form of TLR14 (e.g., a truncated or variant form of the TLR14, e.g., comprising about amino acids 1 to 616 of FIG. 2 (SEQ ID NO:2)) that is capable of forming an inhibitory complex with TLR14). Methods for generating TLR14 propeptides and variants thereof are known in the art and are disclosed in US 2006/0024783, the contents of which are incorporated by reference herein.

### Soluble TLR14

A soluble form of a TLR14 antagonist can be used alone or functionally linked (e.g., by chemical coupling, genetic or polypeptide fusion, non-covalent association or otherwise) to a second moiety, e.g., an immunoglobulin Fc domain, serum albumin, pegylation, a GST, Lex-A or an MBP polypeptide sequence. As used herein, a “fusion protein” refers to a protein containing two or more operably associated, e.g., linked, moieties, e.g., protein moieties. Typically, the moieties are covalently associated. The moieties can be directly associate, or connected via a spacer or linker.

The fusion proteins may additionally include a linker sequence joining the first moiety, e.g., a soluble TLR14, to the second moiety. For example, the fusion protein can include a peptide linker, e.g., a peptide linker of about 4 to 20, more preferably, 5 to 10, amino acids in length; the peptide linker is 8 amino acids in length. Each of the amino acids in the peptide linker is selected from the group consisting of Gly, Ser, Asn, Thr and Ala; the peptide linker includes a Gly-Ser element. In some embodiments, the fusion protein includes a peptide linker and the peptide linker includes a sequence having the formula (Ser-Gly-Gly-Gly-Gly)y wherein y is 1, 2, 3, 4, 5, 6, 7, or 8.

In some embodiments, additional amino acid sequences can be added to the N- or C-terminus of the fusion protein to facilitate expression, detection and/or isolation or purification. For example, TLR14 fusion protein may be linked to one or more additional moieties, e.g., GST, His6 tag, FLAG tag. For example, the fusion protein may additionally be linked to a GST fusion protein in which the fusion protein sequences are fused to the C-terminus of the GST (i.e., glutathione S-transferase) sequences. Such fusion proteins can facilitate the purification of the TLR14 fusion protein.

In some embodiments, the fusion protein is includes a heterologous signal sequence (i.e., a polypeptide sequence that is not present in a polypeptide encoded by a TLR14 nucleic acid) at its N-terminus. For example, in some embodiments, the native TLR14 signal sequence can be removed and replaced with a signal sequence from another protein. In certain host cells (e.g., mammalian host cells), expression and/or secretion of TLR14 can be increased through use of a heterologous signal sequence.

A chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In some embodiments, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) Current Protocols in Molecular Biology, John Wiley & Sons, 1992). Moreover, many expression vectors are commercially available that encode a fusion moiety (e.g., an Fc region of an immunoglobulin heavy chain). A TLR14 encoding nucleic acid can be cloned into such an expression vector such that the fusion moiety is linked in-frame to the immunoglobulin protein.

In some embodiments, TLR14 fusion polypeptides exist as oligomers, such as dimers or trimers.

In some embodiments, the TLR14 polypeptide moiety is provided as a variant TLR14 polypeptide having a mutation in the naturally-occurring TLR14 sequence (wild type) that results in higher affinity (relative to the non-mutated sequence) binding of the TLR14 polypeptide to a TLR14 associated protein, a ligand or a co-receptor.

In some embodiments, the TLR14 polypeptide moiety is provided as a variant TLR14 polypeptide having mutations in the naturally-occurring TLR14 polypeptide sequence (wild type) that results in a TLR14 sequence more resistant to proteolysis (relative to the non-mutated sequence).

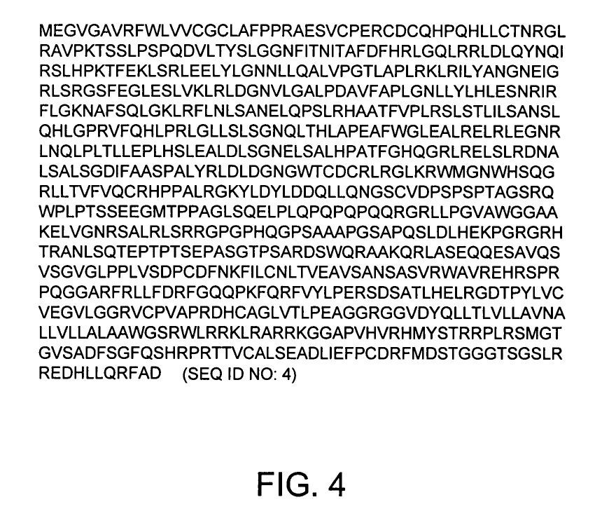

In some embodiments, the first polypeptide includes full-length TLR14 polypeptide. Alternatively, the first polypeptide comprise less than full-length TLR14 polypeptide. For example, the antagonist can be a soluble form of a TLR14 (e.g., a soluble form of an extracellular domain of mammalian (e.g., human) TLR14). For example, the TLR14 antagonist can include about amino acids 1 to 697 of human TLR14 (FIG. 2; SEQ ID NO: 2); or about amino acids 1 to 697 of murine TLR14 (FIG. 4; SEQ ID NO: 4).

In some embodiments, additional amino acid sequences can be added to the N- or C-terminus of the fusion protein to facilitate expression, steric flexibility, detection and/or isolation or purification. The second polypeptide is preferably soluble. In some embodiments, the second polypeptide enhances the half-life, (e.g., the serum half-life) of the linked polypeptide. In some embodiments, the second polypeptide includes a sequence that facilitates association of the fusion polypeptide with a second TLR14 polypeptide. In some embodiments, the second polypeptide includes at least a region of an immunoglobulin polypeptide. Immunoglobulin fusion polypeptides are known in the art and are described in e.g., U.S. Pat. Nos. 5,516,964; 5,225,538; 5,428,130; 5,514,582; 5,714,147; and 5,455,165. For example, a soluble form of a TLR14 or a TLR14 antagonistic propeptide can be fused to a heavy chain constant region of the various isotypes, including: IgG1, IgG2, IgG3, IgG4, IgM, IgA1, IgA2, IgD, and IgE). Typically, the fusion protein can include the extracellular domain of a human TLR14 and, e.g., fused to, a human immunoglobulin Fc chain, e.g., human IgG (e.g., human IgG1 or human IgG2, or a mutated form thereof).

The Fc sequence can be mutated at one or more amino acids to reduce effector cell function, Fc receptor binding and/or complement activity. Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. No. 5,624,821 and U.S. Pat. No. 5,648,260). Similar type of alterations could be described which if applied to the murine, or other species immunoglobulin would reduce or eliminate these functions. For example, it is possible to alter the affinity of an Fc region of an antibody (e.g., an IgG, such as a human IgG) for an FcR (e.g., Fc gamma R1), or for C1q binding by replacing the specified residue(s) with a residue(s) having an appropriate functionality on its side chain, or by introducing a charged functional group, such as glutamate or aspartate, or perhaps an aromatic non-polar residue such as phenylalanine, tyrosine, tryptophan or alanine (see e.g., U.S. Pat. No. 5,624,821).

In some embodiments, the second polypeptide has less effector function that the effector function of a Fc region of a wild-type immunoglobulin heavy chain. Fc effector function includes for example, Fc receptor binding, complement fixation and T cell depleting activity (see for example, U.S. Pat. No. 6,136,310). Methods for assaying T cell depleting activity, Fc effector function, and antibody stability are known in the art. In some embodiments, the second polypeptide has low or no detectable affinity for the Fc receptor. In an alternative embodiment, the second polypeptide has low or no detectable affinity for complement protein C1q.

The antibody molecules and soluble receptor and/or fusion proteins described herein can be functionally linked (e.g., by chemical coupling, genetic fusion, non-covalent association or otherwise) to one or more other molecular entities, such as an antibody (e.g., a bispecific or a multispecific antibody), toxins, radioisotopes, cytotoxic or cytostatic agents, among others.

### TLR14 Variant Molecules and Small Molecules

In yet another embodiment, the TLR14 modulator, e.g., binding agent, antagonist is a variant molecule or a small molecule. An example of a variant molecule typically includes a binding domain polypeptide that is fused or otherwise connected to a hinge or hinge-acting region polypeptide, which in turn is fused or otherwise connected to a region comprising one or more native or engineered constant regions from a heavy chain, other than CH1, for example, the CH2 and CH3 regions of IgG and IgA, or the CH3 and CH4 regions of IgE (see e.g., U.S. Ser. No. 05/0,136,049 by Ledbetter, J. et al. for a more complete description). The binding domain-fusion protein can further include a region that includes a native or engineered heavy chain CH2 constant region polypeptide (or CH3 in the case of a construct derived in whole or in part from IgE) that is fused or otherwise connected to the hinge region polypeptide and a native or engineered heavy chain CH3 constant region polypeptide (or CH4 in the case of a construct derived in whole or in part from IgE) that is fused or otherwise connected to the CH2 constant region polypeptide (or CH3 in the case of a construct derived in whole or in part from IgE). Typically, such binding domain-fusion proteins are capable of at least one activity selected from the group consisting of fusion protein-dependent cell-mediated cytotoxicity, complement fixation, and/or binding to a target, for example, a TLR14.

Typically, the variant or small molecule will bind to a mammalian, e.g., human, TLR14 with an affinity of at least about 10⁷ M⁻¹, typically about 10⁸M⁻¹, and more typically, about 10⁹ M⁻¹ to 10¹⁰ M⁻¹ or stronger; and will reduce and/or inhibit one or more TLR14 biological activities, as described herein. In some embodiments, the variant or small molecule binds to a TLR14 sequence (e.g., a TLR14 sequence comprising the extracellular domain of TLR14 (e.g., In one embodiment, the antibody molecule binds to TLR14 polypeptide, or a portion thereof, e.g., the extracellular domain of TLR14 (e.g., about amino acids 1 to 695 or 696, or a fragment thereof, e.g., about amino acids 85 to 103, or 509 to 526, of FIG. 2 (SEQ ID NO:2). In other embodiments, the antibody molecule binds to a Leucine-rich domain of TLR14, e.g., a domain located at about amino acids 26 to 58, 82 to 105, 106 to 129, 130 to 153, 154 to 177, 178 to 201, 202 to 227, 228 to 251, 252 to 275, 276 to 299, 300 to 323, 324 to 347, or 359 to 315, of FIG. 2 (SEQ ID NO:2). In yet another embodiment, the antibody molecule binds to a fibronectin-type III domain (FN3) located at about amino acids 589 to 671, of FIG. 2 (SEQ ID NO:2), and/or the RGD integrin binding site located at about amino acids 640 to 660, 645 to 655, or 652 to 654, of FIG. 2 (SEQ ID NO:2). Human TLR14 protein may include a leader sequence about 20-21 amino acids long corresponding to about amino acids 1 to 20 of SEQ ID NO:2. In yet other embodiments, the variant or small molecule binds to an intracellular domain of TLR14, e.g., at about amino acids 720 to 811, or a portion thereof (e.g., about amino acids 796 to 811, of FIG. 2 (SEQ ID NO:2)). In yet other embodiments, the variant or small molecule competes for binding to TLR14 with any of antibodies 0540 (A), 0541 (B), and 0547 (C) describes herein (e.g., the antibody molecule binds to an epitope on TLR14 that is the same or substantially similar to the one recognized by antibodies 0540 (A), 0541 (B), and 0547 (C). In other embodiments, the variant or small molecule binds to an epitope as described herein and inhibits, reduces or neutralizes one or more activities of TLR14.

In certain embodiments, the variant or small molecule binds to a TLR14-interacting polypeptide, e.g., NgR, e.g., a human NgR; or p75^{NTR}, e.g., a human p75^{NTR} (e.g., an NgR, or p75^{NTR} comprising an amino acid sequence identical to a mammalian, e.g., human, NgR, or p75^{NTR} as shown in FIG. 22B (SEQ ID NO:8) or FIG. 24B (SEQ ID NO:12), respectively), or a sequence substantially homologous thereto, and inhibits, reduces or neutralizes one or more activities of TLR14.

### Other TLR14 Antagonists

In another embodiment, the TLR14 antagonist is a naturally-occurring antagonists, or a functional fragment or variant thereof. In embodiments, the antagonist is an RGD peptide/peptidomimetic. Examples of RGD peptides/peptidomimetics are described in Dunehoo, A. et al. (2006) Journal of Pharmaceutical Sciences 95(9);1856-1867).

In yet another embodiment, the TLR14 antagonist inhibits the expression of nucleic acid encoding TLR14 or a TLR14-associated polypeptide, e.g., NgR1or p75^{NTR}. Examples of such TLR14 antagonists include nucleic acid molecules, for example, antisense molecules, ribozymes, RNAi, small interfering RNA (siRNA), short hairpin RNA (shRNA), triple helix molecules that hybridize to a nucleic acid encoding TLR14, or a transcription regulatory region, and blocks or reduces mRNA expression of TLR14.

In some embodiments, nucleic acid antagonists are used to decrease expression of an endogenous gene encoding TLR14. In some embodiments, the nucleic acid antagonist is an siRNA that targets mRNA encoding TLR14. Other types of antagonistic nucleic acids can also be used, e.g., shRNA, dsRNA, a ribozyme, a triple-helix former, or an antisense nucleic acid. Accordingly, isolated nucleic acid molecules that are nucleic acid inhibitors, e.g., antisense, RNAi, to TLR14-encoding nucleic acid molecule are provided.

An “antisense” nucleic acid can include a nucleotide sequence which is complementary to a “sense” nucleic acid encoding a protein, e.g., complementary to the coding strand of a double-stranded cDNA molecule or complementary to an mRNA sequence. The antisense nucleic acid can be complementary to an entire TLR14 coding strand, or to only a portion thereof. In some embodiments, the antisense nucleic acid molecule is antisense to a “noncoding region” of the coding strand of a nucleotide sequence encoding TLR14 (e.g., the 5′ and 3′ untranslated regions). Anti-sense agents can include, for example, from about 8 to about 80 nucleobases (i.e. from about 8 to about 80 nucleotides), e.g., about 8 to about 50 nucleobases, or about 12 to about 30 nucleobases. Anti-sense compounds include ribozymes, external guide sequence (EGS) oligonucleotides (oligozymes), and other short catalytic RNAs or catalytic oligonucleotides which hybridize to the target nucleic acid and modulate its expression. Anti-sense compounds can include a stretch of at least eight consecutive nucleobases that are complementary to a sequence in the target gene. An oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

Hybridization of antisense oligonucleotides with mRNA can interfere with one or more of the normal functions of mRNA. The functions of mRNA to be interfered with include all key functions such as, for example, translocation of the RNA to the site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA.

Exemplary antisense compounds include DNA or RNA sequences that specifically hybridize to the target nucleic acid, e.g., the mRNA encoding TLR14. The complementary region can extend for between about 8 to about 80 nucleobases. The compounds can include one or more modified nucleobases. Modified nucleobases may include, e.g., 5-substituted pyrimidines such as 5-iodouracil, 5-iodocytosine, and C5-propynyl pyrimidines such as C5-propynylcytosine and C5-propynyluracil. Other suitable modified nucleobases include N⁴—(C₁-C₁₂)alkylaminocytosines and N⁴,N⁴—(C₁-C₁₂)dialkylaminocytosines. Modified nucleobases may also include 7-substituted-8-aza-7-deazapurines and 7-substituted-7-deazapurines such as, for example, 7-iodo-7-deazapurines, 7-cyano-7-deazapurines, 7-aminocarbonyl-7-deazapurines. Examples of these include 6-amino-7-iodo-7-deazapurines, 6-amino-7-cyano-7-deazapurines, 6-amino-7-aminocarbonyl-7-deazapurines, 2-amino-6-hydroxy-7-iodo-7-deazapurines, 2-amino-6-hydroxy-7-cyano-7-deazapurines, and 2-amino-6-hydroxy-7-aminocarbonyl-7-deazapurines. Furthermore, N⁶—(C₁-C₁₂)alkylaminopurines and N⁶,N⁶—(C₁-C₁₂)dialkylaminopurines, including N⁶-methylaminoadenine and N⁶,N⁶-dimethylaminoadenine, are also suitable modified nucleobases. Similarly, other 6-substituted purines including, for example, 6-thioguanine may constitute appropriate modified nucleobases. Other suitable nucleobases include 2-thiouracil, 8-bromoadenine, 8-bromoguanine, 2-fluoroadenine, and 2-fluoroguanine. Derivatives of any of the aforementioned modified nucleobases are also appropriate. Substituents of any of the preceding compounds may include C₁-C₃₀ alkyl, C₂-C₃₀ alkenyl, C₂-C₃₀ alkynyl, aryl, aralkyl, heteroaryl, halo, amino, amido, nitro, thio, sulfonyl, carboxyl, alkoxy, alkylcarbonyl, alkoxycarbonyl, and the like. Descriptions of other types of nucleic acid agents are also available. See, e.g., U.S. Pat. Nos. 4,987,071; 5,116,742; and 5,093,246; Woolf et al. (1992) Proc Natl Acad Sci USA; Antisense RNA and DNA, D. A. Melton, Ed., Cold Spring Harbor Laboratory, Cold Spring Harbor, N.Y. (1988); 89:7305-9; Haselhoff and Gerlach (1988) Nature 334:585-59; Helene, C. (1991) Anticancer Drug Des. 6:569-84; Helene (1992) Ann. N.Y. Acad. Sci. 660:27-36; and Maher (1992) Bioassays 14:807-15.

The antisense nucleic acid molecules of the invention are typically administered to a subject (e.g., by direct injection at a tissue site), or generated in situ such that they hybridize with or bind to cellular mRNA and/or genomic DNA encoding a TLR14 protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

siRNAs are small double stranded RNAs (dsRNAs) that optionally include overhangs. For example, the duplex region of an siRNA is about 18 to 25 nucleotides in length, e.g., about 19, 20, 21, 22, 23, or 24 nucleotides in length. Typically, the siRNA sequences are exactly complementary to the target mRNA. dsRNAs and siRNAs in particular can be used to silence gene expression in mammalian cells (e.g., human cells). siRNAs also include short hairpin RNAs (shRNAs) with 29-base-pair stems and 2-nucleotide 3′ overhangs. See, e.g., Clemens et al. (2000) Proc. Natl. Acad. Sci. USA 97:6499-6503; Billy et al. (2001) Proc. Natl. Sci. USA 98:14428-14433; Elbashir et al. (2001) Nature. 411:494-8; Yang et al. (2002) Proc. Natl. Acad. Sci. USA 99:9942-9947; Siolas et al. (2005), Nat. Biotechnol. 23(2):227-31; 20040086884; U.S. 20030166282; 20030143204; 20040038278; and 20030224432.

In some embodiments, an antisense nucleic acid of the invention is a ribozyme. A ribozyme having specificity for TLR14-encoding nucleic acid can include one or more sequences complementary to the nucleotide sequence of TLR14 cDNA disclosed herein (i.e., SEQ ID NO:1 or SEQ ID NO:3), and a sequence having known catalytic sequence responsible for mRNA cleavage (see U.S. Pat. No. 5,093,246 or Haselhoff and Gerlach (1988) Nature 334:585-591). For example, a derivative of a Tetrahymena L-19 IVS RNA can be constructed in which the nucleotide sequence of the active site is complementary to the nucleotide sequence to be cleaved in a TLR14-encoding mRNA. See, e.g., Cech et al. U.S. Pat. No. 4,987,071; and Cech et al. U.S. Pat. No. 5,116,742. Alternatively, TLR14 mRNA can be used to select a catalytic RNA having a specific ribonuclease activity from a pool of RNA molecules. See, e.g., Bartel, D. and Szostak, J. W. (1993) Science 261:1411-1418.

TLR14 gene expression can be inhibited by targeting nucleotide sequences complementary to the regulatory region of the TLR14 receptor (e.g., the TLR14 promoter and/or enhancer regions) to form triple helical structures that prevent transcription of the TLR14 gene in target cells. See generally, Helene, C. (1991) Anticancer Drug Des. 6:569-84; Helene, C. i (1992) Ann. N.Y. Acad. Sci. 660:27-36; and Maher, L. J. (1992) Bioassays 14:807-15. The potential sequences that can be targeted for triple helix formation can be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

The invention also provides detectably labeled oligonucleotide primer and probe molecules. Typically, such labels are chemiluminescent, fluorescent, radioactive, or colorimetric.

A TLR14 nucleic acid molecule can be modified at the base moiety, sugar moiety or phosphate backbone to improve, e.g., the stability, hybridization, or solubility of the molecule. For non-limiting examples of synthetic oligonucleotides with modifications see Toulmé (2001) Nature Biotech. 19:17 and Faria et al. (2001) Nature Biotech. 19:40-44. Such phosphoramidite oligonucleotides can be effective antisense agents.

For example, the deoxyribose phosphate backbone of the nucleic acid molecules can be modified to generate peptide nucleic acids (see Hyrup B. et al. (1996) Bioorganic & Medicinal Chemistry 4: 5-23). As used herein, the terms “peptide nucleic acid” or “PNA” refers to a nucleic acid mimic, e.g., a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. The synthesis of PNA oligomers can be performed using standard solid phase peptide synthesis protocols as described in Hyrup B. et al. (1996) supra and Perry-O'Keefe et al. Proc. Natl. Acad. Sci. 93: 14670-675.

PNAs of TLR14 nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of TLR14 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. et al. (1996) supra)); or as probes or primers for DNA sequencing or hybridization (Hyrup B. et al. (1996) supra; Perry-O'Keefe supra).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; W088/09810) or the blood-brain barrier (see, e.g., W0 89/10134). In addition, oligonucleotides can be modified with hybridization-triggered cleavage agents (see, e.g., Krol et al. (1988) Bio-Techniques 6:958-976) or intercalating agents (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

### Recombinant Expression Vectors, Host Cells and Genetically Engineered Cells

In another aspect, the invention includes vectors, preferably expression vectors, containing a nucleic acid encoding polypeptides described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a TLR14 nucleic acid in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., TLR14 proteins, mutant forms of TLR14 proteins, fusion proteins, and the like).

The term “recombinant host cell” (or simply “host cell”), as used herein, is intended to refer to a cell into which a recombinant expression vector has been introduced. It should be understood that such terms are intended to refer not only to the particular subject cell, but to the progeny of such a cell. Because certain modifications may occur in succeeding generations due to either mutation or environmental influences, such progeny may not, in fact, be identical to the parent cell, but are still included within the scope of the term “host cell” as used herein.

The recombinant expression vectors of the invention can be designed for expression of TLR14 proteins in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be used in TLR14 activity assays, (e.g., direct assays or competitive assays described in detail below), or to generate antibodies specific for (i.e., against) TLR14 proteins. In a preferred embodiment, a fusion protein expressed in a retroviral expression vector of the present invention can be used to infect bone marrow cells which are subsequently transplanted into irradiated recipients. The pathology of the subject recipient is then examined after sufficient time has passed (e.g., six weeks).

To maximize recombinant protein expression in E. coli is to express the protein in a host bacteria with an impaired capacity to proteolytically cleave the recombinant protein (Gottesman, S., (1990) Gene Expression Technology: Methods in Enzymology 185, Academic Press, San Diego, Calif. 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

The TLR14 expression vector can be a yeast expression vector, a vector for expression in insect cells, e.g., a baculovirus expression vector or a vector suitable for expression in mammalian cells.

When used in mammalian cells, the expression vector's control functions can be provided by viral regulatory elements. For example, commonly used promoters are derived from polyoma, Adenovirus 2, cytomegalovirus and Simian Virus 40.

In another embodiment, the promoter is an inducible promoter, e.g., a promoter regulated by a steroid hormone, by a polypeptide hormone (e.g., by means of a signal transduction pathway), or by a heterologous polypeptide (e.g., the tetracycline-inducible systems, “Tet-On” and “Tet-Off”; see, e.g., Clontech Inc., CA, Gossen and Bujard (1992) Proc. Natl. Acad. Sci. USA 89:5547, and Paillard (1989) Human Gene Therapy 9:983).

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus.

Another aspect the invention provides a host cell which includes a nucleic acid molecule described herein, e.g., a TLR14 nucleic acid molecule within a recombinant expression vector or a TLR14 nucleic acid molecule containing sequences which allow it to homologously recombine into a specific site of the host cell's genome. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a TLR14 protein can be expressed in bacterial cells (such as E. coli), insect cells, yeast or mammalian cells (such as Chinese hamster ovary cells (CHO) or COS cells e.g., COS-7 cells, CV-1 origin SV40 cells; Gluzman (1981) Cell 23:175-182). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell of the invention can be used to produce (i.e., express) a TLR14 protein. Accordingly, the invention further provides methods for producing a TLR14 protein using the host cells of the invention. In some embodiments, the methods include producing (i.e., expressing) full-length TLR14 using the host cells of the invention. In some embodiments, the methods include producing (i.e., expressing) only a soluble TLR14 domain. In some embodiments, the methods include producing (i.e., expressing) a TLR14 ectodomain and/or a TLR14 transmembrane domain. In some embodiments, the methods include producing (i.e., expressing) a TLR14 antigenic fragment, e.g., a TLR14 fragment that is capable of interaction with an antibody.

In some embodiments, the method includes culturing the host cell of the invention (into which a recombinant expression vector encoding a TLR14 protein has been introduced) in a suitable medium such that a TLR14 protein is produced. In another embodiment, the method further includes isolating a TLR14 protein from the medium or the host cell.

In another aspect, the invention features, a cell or purified preparation of cells which include a TLR14 transgene, or which otherwise misexpress TLR14. The cell preparation can consist of human or non-human cells, e.g., rodent cells, e.g., mouse or rat cells, rabbit cells, or pig cells. In preferred embodiments, the cell or cells include a TLR14 transgene, e.g., a heterologous form of a TLR14, e.g., a gene derived from humans (in the case of a non-human cell). The TLR14 transgene can be misexpressed, e.g., overexpressed or underexpressed. In other preferred embodiments, the cell or cells include a gene that mis-expresses an endogenous TLR14, e.g., a gene the expression of which is disrupted, e.g., a knockout. Such cells can serve as a model for studying disorders that are related to mutated or mis-expressed TLR14 alleles, or for use in drug screening.

Also provided are cells, preferably human cells, e.g., fibroblast cells, in which an endogenous TLR14 is under the control of a regulatory sequence that does not normally control the expression of the endogenous TLR14 gene. The expression characteristics of an endogenous gene within a cell, e.g., a cell line or microorganism, can be modified by inserting a heterologous DNA regulatory element into the genome of the cell such that the inserted regulatory element is operably linked to the endogenous TLR14 gene. For example, an endogenous TLR14 gene which is “transcriptionally silent,” e.g., not normally expressed, or expressed only at very low levels, may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published in May 16, 1991.

In some embodiments, the implanted recombinant cells express and secrete an antibody specific for a TLR14/TLR14-associated protein polypeptide. The antibody can be any antibody or any antibody derivative described herein.

### Screening Assays

The invention provides methods (also referred to herein as “screening assays”) for identifying modulators, i.e., candidate or test compounds or agents (e.g., proteins, peptides, peptidomimetics, peptoids, small molecules or other drugs) which bind to TLR14 proteins, have an inhibitory effect on, for example, TLR14 expression or TLR14 activity, or have an inhibitory effect on, for example, the expression or activity of a TLR14-associated protein, e.g., a ligand, co-receptor, or a neuronal protein as described herein, e.g., p75^{NTR} or NgR1. Compounds thus identified can be used to modulate the activity of target gene products (e.g., TLR14 genes) in a therapeutic protocol, to elaborate the biological function of the target gene product, or to identify compounds that disrupt normal target gene interactions. In some embodiments, the invention provides assays for screening candidate or test compounds that bind to or modulate an activity of a TLR14 protein or polypeptide or a biologically active portion thereof

Accordingly, the invention provides a method, or an assay, for identifying a compound, e.g., a test compound, that modulates TLR14. The method, or the assay, includes: (i) (optionally) providing or identifying a test agent that interacts with, e.g., binds to, TLR14; and/or (ii) evaluating a change in an activity of a TLR14-expressing cell (e.g., a cell that tests positive for TLR14 expression using, for example, the antibodies described herein, and other neuronal cells including but not limited to, for example, a neuron, a neural progenitor, a neural stem cell, a multipotent or pluripotent cell, a glia cell, a microglia, an astrocyte, and an oligodendrocyte) in the presence of the test agent, relative to a reference, e.g., a reference sample.

The test compound or agent can be an antibody molecule; peptide; a soluble TLR14 or a fusion thereof; a variant molecule; a small molecule, e.g., a member of a combinatorial or natural product library; a nucleic acid; an antisense molecule; a ribozyme; an RNAi; a triple helix molecule; or any combination thereof. In some embodiments, the test compound antagonizes (e.g., decreases) the activity or expression of a TLR14 polypeptide or nucleic acid. For example, the expression of the TLR14 nucleic acid can be antagonized by e.g., altering mRNA transcription, mRNA stability, etc.

In some embodiments, the evaluating step includes contacting one or more of: a TLR14 polypeptide (e.g., a TLR14 as described herein), or a nucleic acid encoding TLR14, with the test compound; and evaluating a change in one or more activities of the TLR14 polypeptide or nucleic acid, in the presence of the test compound, relative to a predetermined level, e.g., a control sample without the test compound. The contacting step can be effected in vitro (in cultured cells, e.g., neuronal or glial cells, or a reconstituted system) or in vivo (e.g., by administering the test compound to a non-human subject, e.g., an animal model having a mutation in a TLR14 gene). The contacting step(s) and/or the administration of the test compound can be repeated.

In some embodiments, an interaction between the test compound and TLR14 is evaluated. In some embodiments, such interaction can be evaluated by detecting a change in the formation and/or stability of the complex between the test compound and TLR14 can be determined by detecting one or more of a change in the binding or physical formation of the complex itself, e.g., by biochemical detection, affinity based detection (e.g., Western blot, affinity columns), immunoprecipitation, fluorescence resonance energy transfer (FRET)-based assays, spectrophotometric means (e.g., circular dichroism, absorbance, and other measurements of solution properties); a change, e.g., increase or decrease, in signal transduction, e.g., p38 phosphorylation, IkB degradation and/or transcription activity of a TLR14-associated gene; and/or (vii) a change, e.g., increase or decrease in neurite outgrowth.

In one embodiment, the test compound is identified and re-tested in the same or a different assay. For example, a test compound is identified in an in vitro or cell-free system, and re-tested in an animal model or a cell-based assay. Any order or combination of assays can be used. For example, a high throughput assay can be used in combination with an animal model or tissue culture.

The test compounds of the present invention can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In some embodiments, an assay is a cell-based assay in which a cell which expresses a TLR14 protein or biologically active portion thereof is contacted with a test compound, and the ability of the test compound to antagonize TLR14 activity is determined. Determining the ability of the test compound to antagonize TLR14 activity can be accomplished by monitoring, for example, neurite outgrowth, activation of RhoA, PKC, CdC42, Rac1, PAK1, ROCK, LIM-kinase, cofilin, actin, cAMP levels, ERK activation, NF-kB, transcriptional activation of a large family of immune genes such as TNFa, IL-1b, MCP-1, IL-12 and the production of NO.

In some embodiments, a cell-free assay is provided in which a TLR14 protein or biologically active portion thereof is contacted with a test compound and the ability of the test compound to bind to the TLR14 protein or biologically active portion thereof is evaluated. Preferred biologically active portions of the TLR14 proteins to be used in assays of the present invention include fragments which participate in interactions with non-TLR14 molecules, e.g., fragments with high surface probability scores.

Soluble and/or membrane-bound forms of isolated proteins (e.g., TLR14 proteins or biologically active portions thereof) can be used in the cell-free assays of the invention. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence resonance energy transfer (FRET). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A FRET binding event can be conveniently measured through standard fluorometric detection means known in the art (e.g., using a fluorimeter).

In another embodiment, determining the ability of the TLR14 protein to bind to a target molecule can be accomplished using real-time Biomolecular Interaction Analysis (BIA) (see, e.g., Sjolander, S. and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize either TLR14 or an anti-TLR14 antibody to facilitate separation of complexed from uncomplexed forms of one or both of the proteins, as well as to accommodate automation of the assay. Binding of a test compound to a TLR14 protein, or interaction of a TLR14 protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/TLR14 fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and either the non-adsorbed target protein or TLR14 protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of TLR14 binding or activity determined using standard techniques.

Other techniques for immobilizing either a TLR14 protein or a target molecule on matrices include using conjugation of biotin and streptavidin. Biotinylated TLR14 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with TLR14 protein or target molecules but which do not interfere with binding of the TLR14 protein to its target molecule. Such antibodies can be derivatized to the wells of the plate, and unbound target or TLR14 protein trapped in the wells by antibody conjugation. Methods for detecting such complexes, in addition to those described above for the GST-immobilized complexes, include immunodetection of complexes using antibodies reactive with the TLR14 protein or target molecule, as well as enzyme-linked assays which rely on detecting an enzymatic activity associated with the TLR14/TLR14-associated protein protein or target molecule.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., (1993) Trends Biochem Sci 18:284-7); chromatography (gel filtration chromatography, ion-exchange chromatography); electrophoresis (see, e.g., Ausubel, F. et al., eds. Current Protocols in Molecular Biology 1999, J. Wiley: New York.); and immunoprecipitation (see, for example, Ausubel, F. et al., eds. (1999) Current Protocols in Molecular Biology, J. Wiley: New York). Such resins and chromatographic techniques are known to one skilled in the art (see, e.g., Heegaard, N. H., (1998) J Mol Recognit 11:141-8; Hage, D. S., and Tweed, S. A. (1997) J Chromatogr B Biomed Sci Appl. 699:499-525). Further, fluorescence energy transfer may also be conveniently utilized, as described herein, to detect binding without further purification of the complex from solution.

The target gene products of the invention can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target gene product. Such compounds can include, but are not limited to molecules such as antibodies, peptides, and small molecules. The preferred target genes/products for use in this embodiment are the TLR14 genes herein identified. In an alternative embodiment, the invention provides methods for determining the ability of the test compound to modulate the activity of a TLR14 protein through modulation of the activity of a downstream effector of a TLR14 target molecule. For example, the activity of the effector molecule on an appropriate target can be determined, or the binding of the effector to an appropriate target can be determined, as previously described.

To identify compounds that interfere with the interaction between the target gene product and its cellular or extracellular binding partner(s), a reaction mixture containing the target gene product and the binding partner is prepared, under conditions and for a time sufficient, to allow the two products to form complex. In order to test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

These assays can be conducted in a heterogeneous or homogeneous format. Heterogeneous assays involve anchoring either the target gene product or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner, is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target gene product and the interactive cellular or extracellular binding partner product is prepared in that either the target gene products or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

In yet another aspect, the TLR14 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with TLR14 (“TLR14-binding proteins” or “TLR14-bp”) and are involved in TLR14 activity. Such TLR14-bps can be inhibitors of signals by TLR14 proteins or TLR14 targets as, for example, downstream elements of a TLR14-mediated signaling pathway.

The two-hybrid system is based on the modular nature of most transcription factors, which consist of separable DNA-binding and activation domains. Briefly, the assay utilizes two different DNA constructs. In one construct, the gene that codes for a TLR14 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. (Alternatively the: TLR14 protein can be the fused to the activator domain.) If the “bait” and the “prey” proteins are able to interact, in vivo, forming a TLR14-dependent complex, the DNA-binding and activation domains of the transcription factor are brought into close proximity. This proximity allows transcription of a reporter gene (e.g., lacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the TLR14 protein.

In another embodiment, modulators of TLR14 expression are identified. For example, a cell or cell free mixture is contacted with a candidate compound and the expression of TLR14 mRNA or protein evaluated relative to the level of expression of TLR14 mRNA or protein in the absence of the candidate compound. When expression of TLR14 mRNA or protein is less (statistically significantly less) in the presence of the candidate compound than in its absence, the candidate compound is identified as an inhibitor of TLR14 mRNA or protein expression. The level of TLR14 mRNA or protein expression can be determined by methods described herein for detecting TLR14 mRNA or protein.

In another aspect, the invention pertains to a combination of two or more of the assays described herein. For example, a modulating agent can be identified using a cell-based or a cell free assay, and the ability of the agent to modulate the activity of a TLR14 protein can be confirmed in vivo, e.g., in an animal model.

In general, activity of a given test compound in the nervous system can be assayed by detecting the compound's ability to affect one of more of: promote neurite outgrowth, protect neurons from damage by chemical treatments, promote the growth of neurons or neuronal cells, recover lost or damaged motor, functional or cognitive ability associated with nervous tissue or organs of the nervous system, or regenerate neurons. For example, isolated neuronal cell cultures (e.g., dopaminergic, cortical, DRG cell cultures) can be isolated and cultured by methods known in the art (see e.g., Pong et al. (1997) J. Neurochem. 69:986-994; Pong et al. (2001) Exp Neurol. 171(1):84-97). Changes in neuronal activity, differentiation, survival can be detected and quantified using art recognized techniques as described in, e.g., US 2005/0197356 (describing examples showing measuring changes in 3H-dopamine uptake and neurofilament content in cultured dopaminergic neurons and cortical neurons, respectively). Alternatively, neuronal activities can be characterized in cultured neural cell lines, e.g., neuroblastoma cell lines, pheochromocytoma cells (PC12 cells), F11. Activities in vitro can be useful in identifying agents that can be used to treat and/or ameliorate a number of human neurodegenerative conditions, including but not limited to, Parkinson's disease; Alzheimer's disease; amyotrophic lateral sclerosis (ALS); traumatic brain injury; spinal cord injury; multiple sclerosis; diabetic neuropathy; neuropathy associated with medical treatments such as chemotherapy; ischemia or ischemia-induced injury; stroke, among others.

Methods for detecting neuronal activity include, for example, neuroprotective assays where a compound is tested for its ability to protect against glutamate neurotoxicity. Sensory neuronal cultures (DRG) can also be assayed for neurite outgrowth, and assayed for neurotrophic activity. Cultured cells are treated with an immunophilin ligand and later assayed for the presence of new neurite fibers. Immunohistochemistry can aid in the visualization and quantitation of neurites as compared to control.

Examples of animal models that can be used to evaluate the effects of the test compound include middle cerebral artery occlusion (MCAO) or stroke model; a CNS inflammatory model, e.g., EAE, lymphocytic meningeal encephalitis models; Venezuelan equine encephalitis infection meningeal encephalitis, pneumococous CNS inflammation; or a genetically modified model, e.g., an animal model having overexpressed TLR14 or a mutation in a TLR receptor (e.g., a TLR14 deficient animal).

A number of additional animal models and cell culture assays have been developed and can be relied on for their clinical relevance to disease treatments, including the human diseases noted above. Each of the following references can be used as a source for these assays, and all of them are specifically incorporated herein by reference in their entirety for that purpose: Steiner, et al., Proc. Natl. Acad. Sci. U.S.A. 94: 2019-2024 (1997); Hamilton, et al., Bioorgan. Med. Chem. Lett. 7:1785-1790 (1997); McMahon, et al., Curr. Opin. Neurobiol. 5:616-624 (1995); Gash, et al., Nature 380:252-255 (1996); Gerlach, et al., Eur. J. Pharmacol.—Mol. Pharmacol. 208:273-286 (1991); Apfel, et al., Brain Res. 634:7-12 (1994); Wang, et al., J. Pharmacol. Exp. Therap. 282:1084-1093 (1997); Gold, et al., Exp. Neurol. 147:269-278 (1997); Hoffer et al., J. Neural Transm. [Suppl.] 49:1-10 (1997); and Lyons, et al., PNAS 91:3191-3195 (1994).

This invention further pertains to novel agents identified by the above-described screening assays. Accordingly, it is within the scope of this invention to further use an agent identified as described herein (e.g., a TLR14 antagonizing agent, an antisense TLR14 nucleic acid molecule, a TLR14-specific antibody, or a TLR14-binding partner) in an appropriate animal model to determine the efficacy, toxicity, side effects, or mechanism of action, of treatment with such an agent. Furthermore, novel agents identified by the above-described screening assays can be used for treatments as described herein.

The screening methods of the invention are performed either in vitro (for example by monitoring TLR14 activity in a cell-based assay or in an enzymatic activity assay) or in vivo (for example by monitoring TLR14 activity or expression in tissue samples after administering a test agent to a mammal). Exemplary mammals include without limitation, human, mouse, rat, and dog.

### Diagnostic and Prognostic Assays

In another aspect, the invention provides methods for evaluating, diagnosing, and/or monitoring the progression of, a TLR14-associated neurodegenerative disorder (e.g., a disorder as described herein) in a test sample. The method includes evaluating the expression or activity of a nucleic acid or polypeptide chosen from TLR14-associated gene, such that, a difference in the level of the nucleic acid or polypeptide relative to a reference sample, e.g., a sample obtained from normal subject or prior to treatment, is indicative of the presence or progression of the disorder, wherein the differential expression of the TLR14 protein or gene can be used as a marker to indicate the onset and/or progress of the disease. Exemplary TLR14-associated genes include, but are not limited to, p75^{NTR}, TNF, NO, and MMP-9. In certain embodiments, an increase in the level of TLR14 or a TLR14-associated gene in the test sample, relative to a reference sample, is associated with the diagnosis of TLR14 neurodegenerative disorder where antagonism of TLR14 function is desirable. In other embodiments, a decrease in the level of a TLR14 or a TLR14-associated gene in the test sample, relative to a reference sample, is associated with the diagnosis of TLR14 disorder where activation of TLR14 function is desirable.

In one embodiment, the evaluating step occurs in vitro or ex vivo. For example, a sample, e.g., a serum sample, is obtained from the subject. In certain embodiments, a method of monitoring the progression of a neurodegenerative disease from a first timepoint to a later timepoint is provided. The method includes: measuring the concentration of the TLR14 protein or nucleic acid in a first sample obtained at a first time point; measuring the concentration of TLR14 protein or nucleic acid in a second sample obtained at a later timepoint than the first biological sample; determining the difference in concentration of TLR14 between the first and second samples, wherein a higher concentration at the second timepoint is indicative of the onset or progression of a neurodegenerative disease.

In another embodiment, the evaluating step occurs in vivo. For example, by administering to the subject a detectably labeled agent that interacts with the TLR14, or TLR14 associated, nucleic acid or polypeptide, such that a signal is generated relative to the level of activity or expression of the nucleic acid or polypeptide.

A further aspect of the invention provides for the use of the TLR14 protein as a predictive marker for a neurodegenerative disease.

In a yet further aspect, the invention includes a method for diagnosis a neurodegenerative disease, or the onset of a neurodegenerative disease, in an individual. The method includes:

measuring the amount of a TLR14 protein or nucleic acid in a sample obtained from an individual; and

comparing the amount of a TLR14 protein or nucleic acid to a predetermined value;

wherein the presence of TLR14 protein at an amount greater than a predetermined value is indicative of the onset or development of a neurodegenerative disease.

In one embodiment, the predetermined value is the amount of TLR14 protein or nucleic measured in a sample from the same individual at an earlier timepoint.

In one embodiment the TLR14 protein or nucleic acid in the sample is measured by means as described herein, e.g., radioimmunoassay (RIA), enzyme-linked immunosorbent assay (ELISA), and other techniques of protein or nucleic acid detection described herein.

In a yet further aspect, the invention provides a method for detecting a neurodegenerative disorder or susceptibility to a neurodegenerative disorder in a subject. The method includes:

(a) providing a sample of nucleic acids and/or polypeptides from the subject; and

(b) detecting the presence of differential expression of a TLR14 gene, or a fragment thereof.

In one embodiment the detection step of comprises evaluating the amount of TLR14 protein in the sample.

### Expression Monitoring and Profiling.

The presence, level, or absence of TLR14/TLR14-associated protein (e.g., NgR1 or p75^{NTR}) or nucleic acid in a sample, e.g., a biological sample, can be evaluated by obtaining a sample from a test subject and contacting the biological sample with a compound or an agent capable of detecting TLR14/TLR14-associated protein or nucleic acid (e.g., mRNA, genomic DNA) that encodes TLR14/TLR14-associated protein such that the presence of TLR14/TLR14-associated protein or nucleic acid is detected in the biological sample. The term “biological sample” includes tissues, cells and biological fluids isolated from a subject, as well as tissues, cells and fluids present within a subject. A preferred biological sample is serum. The level of expression of the TLR14/TLR14-associated protein gene can be measured in a number of ways, including, but not limited to: measuring the mRNA encoded by the TLR14/TLR14-associated protein genes; measuring the amount of protein encoded by the TLR14/TLR14-associated protein genes; or measuring the activity of the protein encoded by the TLR14/TLR14-associated protein genes.

The level of mRNA corresponding to the TLR14/TLR14-associated protein gene in a cell can be determined both by in situ and by in vitro formats.

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length TLR14/TLR14-associated protein nucleic acid, such as the nucleic acid of SEQ ID NO:1, or a portion thereof, such as an oligonucleotide of at least 7, 15, 30, 50, 100, 250 or 500 nucleotides in length and sufficient to specifically hybridize under stringent conditions to TLR14/TLR14 associated protein mRNA or genomic DNA. The probe can be disposed on an address of an array, e.g., an array described below. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array described below. A skilled artisan can adapt known mRNA detection methods for use in detecting the level of mRNA encoded by the TLR14/TLR14-associated protein genes.

The level of mRNA in a sample that is encoded by one of TLR14/TLR14-associated can be evaluated with nucleic acid amplification, e.g., by rtPCR (Mullis (1987) U.S. Pat. No. 4,683,202), ligase chain reaction (Barany (1991) Proc. Natl. Acad. Sci. USA 88:189-193), self sustained sequence replication (Guatelli et al., (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al. U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the TLR14/TLR14-associated protein gene being analyzed.

In another embodiment, the method further includes contacting a control sample with a compound or agent capable of detecting TLR14/TLR14-associated protein mRNA, or genomic DNA, and comparing the presence of TLR14/TLR14-associated protein mRNA or genomic DNA in the control sample with the presence of TLR14/TLR14-associated protein mRNA or genomic DNA in the test sample. In still another embodiment, serial analysis of gene expression, as described in U.S. Pat. No. 5,695,937, is used to detect TLR14/TLR14-associated protein transcript levels.

A variety of methods can be used to determine the level of protein encoded by TLR14/TLR14-associated protein. In general, these methods include contacting an agent that selectively binds to the protein, such as an antibody with a sample, to evaluate the level of protein in the sample. In a preferred embodiment, the antibody bears a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect TLR14/TLR14-associated protein protein in a biological sample in vitro as well as in vivo. In vitro techniques for detection of TLR14/TLR14-associated protein protein include enzyme linked immunosorbent assays (ELISAs), immunoprecipitations, immunofluorescence, enzyme immunoassay (EIA), radioimmunoassay (RIA), and Western blot analysis. In vivo techniques for detection of TLR14/TLR14-associated protein include introducing into a subject a labeled anti-TLR14/TLR14-associated protein antibody. For example, the antibody can be labeled with a radioactive marker whose presence and location in a subject can be detected by standard imaging techniques. In another embodiment, the sample is labeled, e.g., biotinylated and then contacted to the antibody, e.g., an anti-TLR14/TLR14-associated protein antibody positioned on an antibody array (as described below). The sample can be detected, e.g., with avidin coupled to a fluorescent label.

In another embodiment, the methods further include contacting the control sample with a compound or agent capable of detecting TLR14/TLR14-associated protein, and comparing the presence of TLR14/TLR14-associated protein in the control sample with the presence of TLR14/TLR14-associated protein in the test sample.

The invention also includes kits for detecting the presence of TLR14/TLR14-associated protein in a biological sample. For example, the kit can include a compound or agent capable of detecting TLR14/TLR14-associated protein or mRNA in a biological sample; and a standard. The compound or agent can be packaged in a suitable container. The kit can further comprise instructions for using the kit to detect TLR14/TLR14-associated protein or nucleic acid.

For antibody-based kits, the kit can include: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent.

For oligonucleotide-based kits, the kit can include: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also includes a buffering agent, a preservative, or a protein stabilizing agent. The kit can also includes components necessary for detecting the detectable agent (e.g., an enzyme or a substrate). The kit can also contain a control sample or a series of control samples which can be assayed and compared to the test sample contained. Each component of the kit can be enclosed within an individual container and all of the various containers can be within a single package, along with instructions for interpreting the results of the assays performed using the kit.

The diagnostic methods described herein can identify subjects having, or at risk of developing, a disease or disorder associated with misexpressed or aberrant or unwanted TLR14/TLR14-associated protein expression or activity. As used herein, the term “unwanted” includes an unwanted phenomenon involved in a biological response such as airway inflammation.

In one embodiment, a disease or disorder associated with aberrant or unwanted TLR14/TLR14-associated protein expression or activity is identified. A test sample is obtained from a subject and TLR14/TLR14-associated protein or nucleic acid (e.g., mRNA or genomic DNA) is evaluated, wherein the level, e.g., the presence or absence, of TLR14/TLR14-associated protein or nucleic acid is diagnostic for a subject having or at risk of developing a disease or disorder associated with aberrant or unwanted TLR14/TLR14-associated protein expression or activity. As used herein, a “test sample” refers to a biological sample obtained from a subject of interest, including a biological fluid (e.g., serum), cell sample, or tissue.

The prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant or unwanted TLR14/TLR14-associated protein expression or activity. For example, such methods can be used to determine whether a subject can be effectively treated with an agent to antagonize or otherwise inhibit TLR14/TLR14-associated protein expression or activity.

In another aspect, the invention features a computer medium having a plurality of digitally encoded data records. Each data record includes a value representing the level of expression of TLR14/TLR14-associated protein in a sample, and a descriptor of the sample. The descriptor of the sample can be an identifier of the sample, a subject from which the sample was derived (e.g., a patient), a diagnosis, or a treatment (e.g., a preferred treatment). In a preferred embodiment, the data record further includes values representing the level of expression of genes other than TLR14/TLR14-associated protein (e.g., other genes associated with a TLR14/TLR14-associated protein-disorder, or other genes on an array). The data record can be structured as a table, e.g., a table that is part of a database such as a relational database (e.g., a SQL database of the Oracle or Sybase database environments).

Also featured is a method of evaluating a sample. The method includes providing a sample, e.g., from the subject, and determining a gene expression profile of the sample, wherein the profile includes a value representing the level of TLR14/TLR14-associated protein expression. The method can further include comparing the value or the profile (i.e., multiple values) to a reference value or reference profile. The gene expression profile of the sample can be obtained by any of the methods described herein (e.g., by providing a nucleic acid from the sample and contacting the nucleic acid to an array). The method can be used to diagnose a airway inflammatory disorder in a subject wherein an increase in TLR14/TLR14-associated protein expression or activity is an indication that the subject has or is disposed to having a chronic airway inflammatory disorder, including, for example, asthma. The method can be used to monitor a treatment for asthma in a subject. For example, the gene expression profile can be determined for a sample from a subject undergoing treatment. The profile can be compared to a reference profile or to a profile obtained from the subject prior to treatment or prior to onset of the disorder (see, e.g., Golub et al. (1999) Science 286:531).

In yet another aspect, the invention features a method of evaluating a test compound (see also, “Screening Assays”, above). The method includes providing a cell (e.g., a neural cell) and a test compound; contacting the test compound to the cell; obtaining a subject expression profile for the contacted cell; and comparing the subject expression profile to one or more reference profiles. The profiles include a value representing the level of TLR14/TLR14-associated protein expression. In a preferred embodiment, the subject expression profile is compared to a target profile, e.g., a profile for a normal cell or for desired condition of a cell. The test compound is evaluated favorably if the subject expression profile is more similar to the target profile than an expression profile obtained from an cell not contacted with the test compound.

In another aspect, the invention features, a method of evaluating a subject. The method includes: a) obtaining a sample from a subject, e.g., from a caregiver, e.g., a caregiver who obtains the sample from the subject; b) determining a subject expression profile for the sample. Optionally, the method further includes either or both of steps: c) comparing the subject expression profile to one or more reference expression profiles; and d) selecting the reference profile most similar to the subject reference profile. The subject expression profile and the reference profiles include a value representing the level of TLR14/TLR14-associated protein expression. A variety of routine statistical measures can be used to compare two reference profiles. One possible metric is the length of the distance vector that is the difference between the two profiles. Each of the subject and reference profile is represented as a multi-dimensional vector, wherein each dimension is a value in the profile.

The method can further include transmitting a result to a caregiver. The result can be the subject expression profile, a result of a comparison of the subject expression profile with another profile, a most similar reference profile, or a descriptor of any of the aforementioned. The result can be transmitted across a computer network, e.g., the result can be in the form of a computer transmission, e.g., a computer data signal embedded in a carrier wave.

Also featured is a computer medium having executable code for effecting the following steps: receive a subject expression profile; access a database of reference expression profiles; and either i) select a matching reference profile most similar to the subject expression profile or ii) determine at least one comparison score for the similarity of the subject expression profile to at least one reference profile. The subject expression profile, and the reference expression profiles each include a value representing the level of TLR14/TLR14-associated protein expression.

### Examples

The Examples that follow are set forth to aid in the understanding of the inventions but are not intended to, and should not be construed to, limit its scope in any way.

### Example 1

### Structural Analysis of Human TLR14

The non-redundant (NR) protein database and the three dimensional structural database (PDB) were searched for homologies between the human TLR14 nucleotide sequence (FIG. 1; SEQ ID NO:1), or protein sequence (FIG. 2; SEQ ID NO:2) and other proteins using BLASTP at the NCBI (http://www.ncbi.nlm.nih.gov).

The homology between TLR14 and NgR1 was principally confined to the first 400 amino acids of the sequence. Phylogenetic analysis was performed using the Phylogeny Inference package (PHYLIP) available from http://evolution.genetics.washington.edu/phylip.html. PHYLIP is a free package of programs for inferring phylogenies. It is distributed as source code, documentation files, and a number of different types of executables. These Web pages, by Joe Felsenstein of the Department of Genome Sciences and the Department of Biology at the University of Washington, contain information on PHYLIP and ways to transfer the executables, source code and documentation to your computer. PHYLIP 3.67 was released on 24 Jul. 2004.

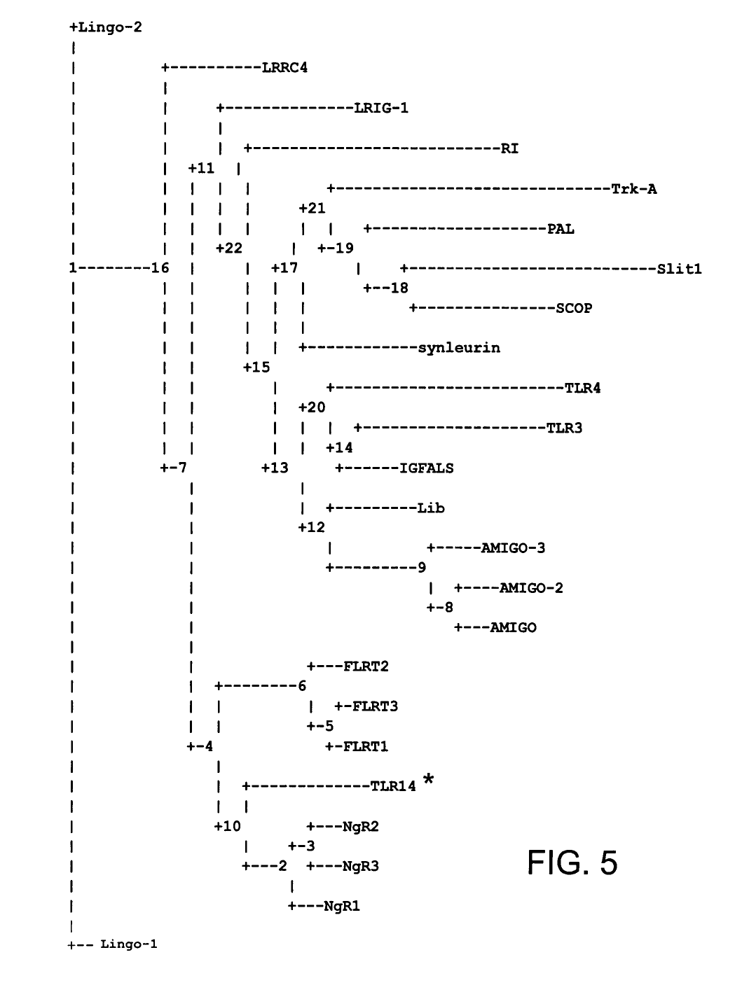

TLR14 is indicated as “*.” Included in this analysis were members of the Nogo-receptor family and other leucine-rich repeat containing proteins. The results from the phylogenetic analysis are shown in FIG. 5. This analysis revealed that TLR14 is closely related to the NgR1 family of neuronal proteins.

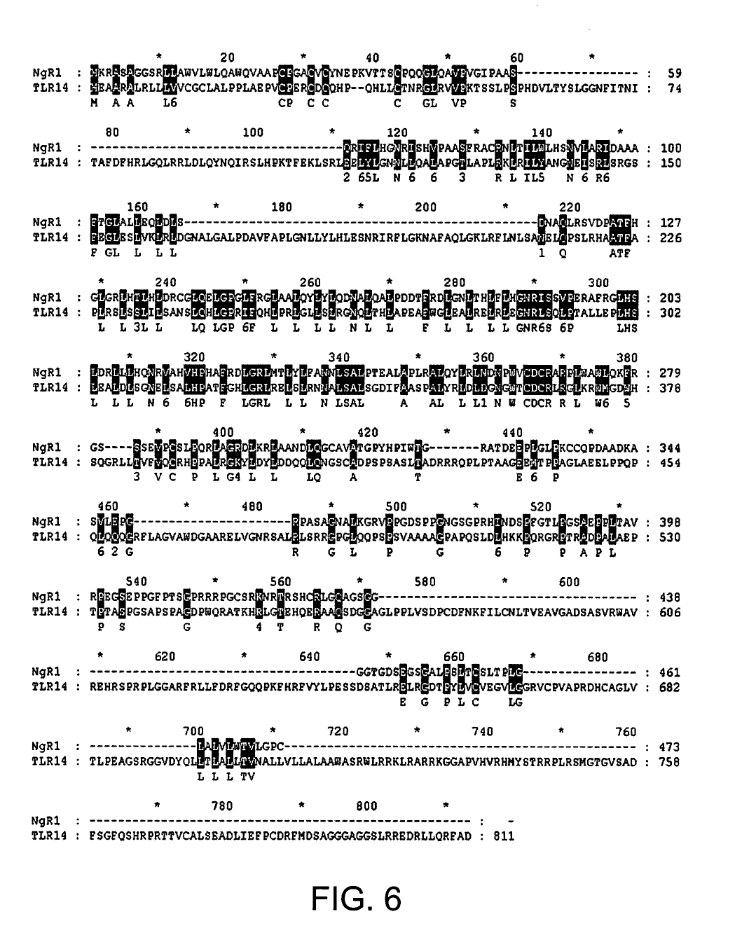

A significant homology (37% identity, 47% homology) was found between TLR14 and NgR1 in the Leucine Rich Repeat (LRR) region. The default parameters of ClustalW (http://www.ebi.ac.uk/clustalW/) were then used to generate alignments between TLR14 and NgR1(Barton et. al. (2003) Embo J. 22, 3291) as shown in FIG. 6.

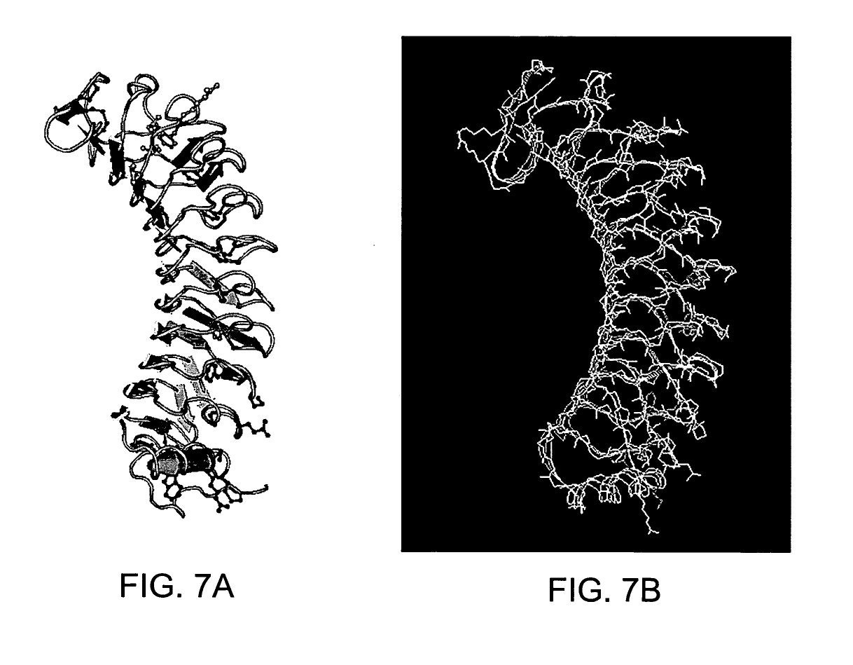

The crystal structure of NgR1 (Barton et. al. (2003) supra) was annotated and viewed using CN3D version 4.1 at the NCBI available from (ncbi.nlm.nih.gov) as shown in FIG. 7A.

A three dimensional model of TLR14 was generated (FIG. 7B) using the automated Swiss-model homology-modelling pipeline referenced via the UniProt database at Expasy (www.expasy.org). The automated model used NgR1 (Acc. no. 10ZNA, 18PT) and Decorin (Acc. No. 1×CDA, 1×ECB) structures as a template. The model was annotated using DeepView Swiss-PDB viewer (http://www.expasy.org/spdbv).

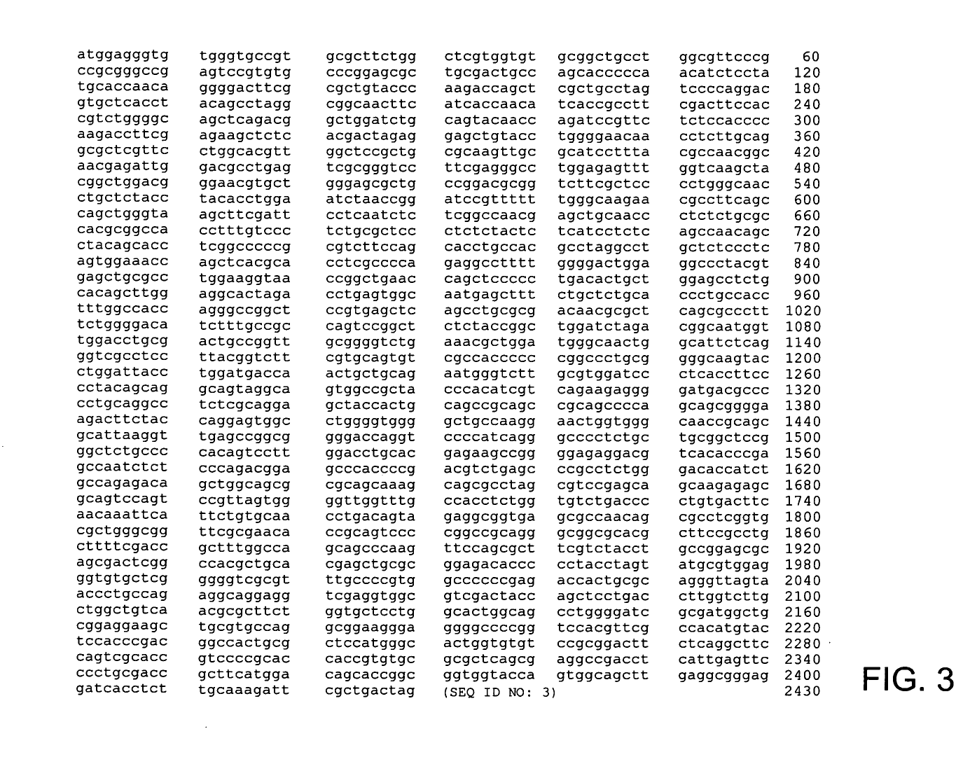

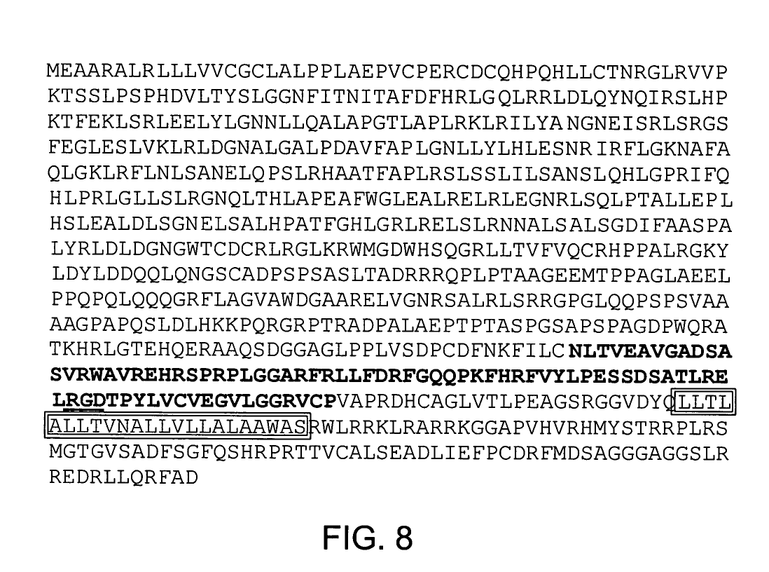

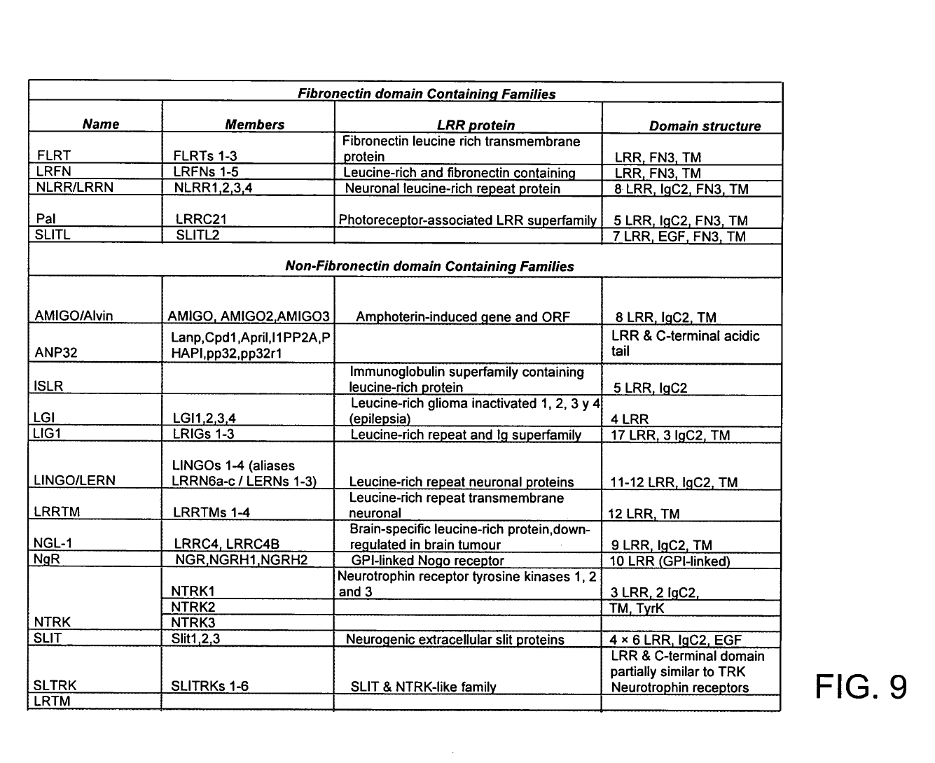

The TLR14 protein nucleotide and amino acid sequence and its murine counter part (FIGS. 3-4 (SEQ ID NOs: 3-4), respectively, for murine nucleotide and amino acid sequence) were shown to contain a leucine rich repeat (LRR) region a Fibronectin III like domain (FN3) and a transmembrane domain (FIG. 8). These domains can be used to classify this protein as being similar to other proteins expressed in the brain that contain LRR and FN3 domains such as: LRFN (leucine rich and fibronectin containing), NLRR/LRRN (Neuronal leucine-rich repeat protein), Pal (Photoreceptor-associated LRR superfamily) and SLITL (SLIT-like 2 (Homo Sapiens Vasorin)) (FIG. 9). There are also a number of other LRR-containing proteins expressed in the brain (FIG. 9).

### Example 2

### Expression of TLR14 mRNA in the Normal and Diseased Brain

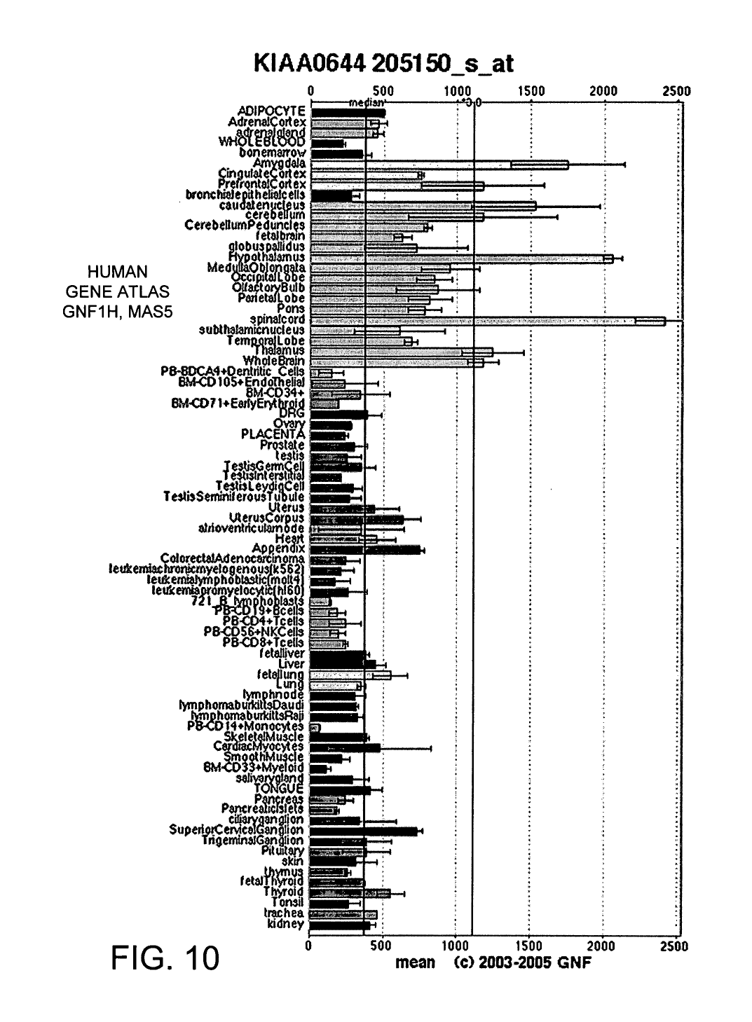

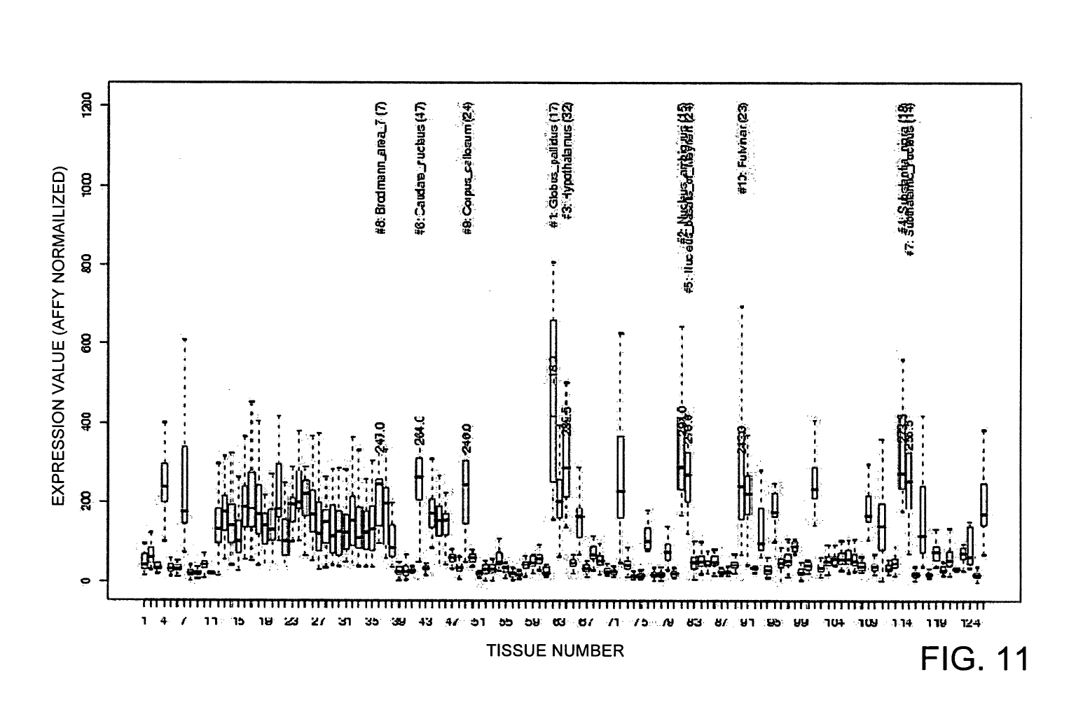

Analysis of TLR14 expression in the brain was carried out on TLR14 using SymAtlas (Su, A. et al. (2002) PNAS 99(7):4465-70). This analysis revealed high neuronal expression as shown in FIG. 10. Additional expression profiling from normal human brain samples further supported this data (FIG. 11).

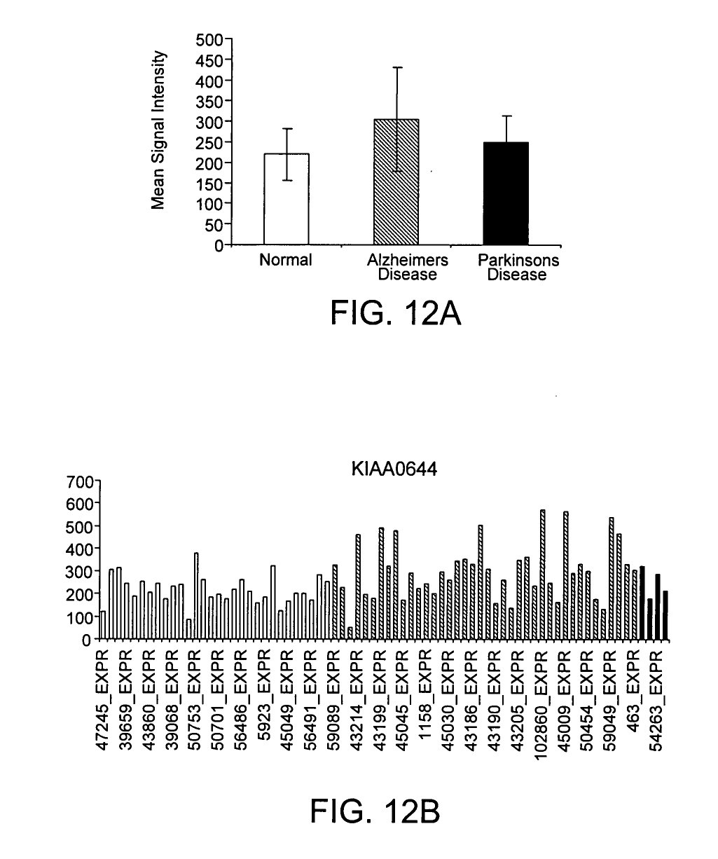

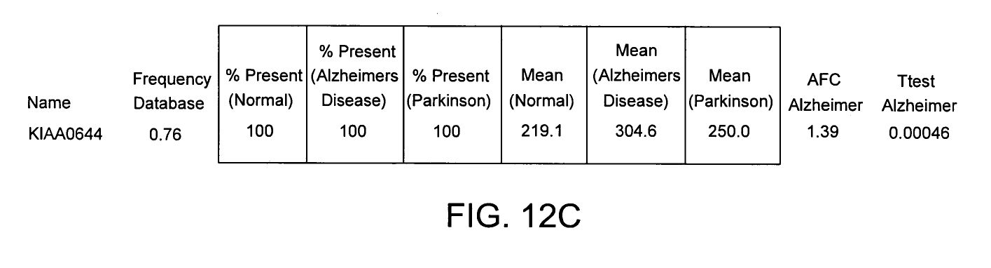

TLR14 mRNA levels were shown to be elevated in Alzheimer's and Parkinson's disease patients (FIG. 12). TLR14 expression was analyzed in various tissues using Affymetrix® Hg_U133 GeneChip™ oligonucleotide microarrays. FIG. 12A shows TLR14 mean±standard deviation expression levels for normal, Alzheimer's, and Parkinson's brain samples measured by GeneChip™ in the genelogic expression database. FIG. 12B shows individual measurements used for (A). FIG. 12C is a tabular summary of the data present in FIGS. 12A and 12B. “Frequency database” indicates the overall frequency of detection for TLR14. “% present” indicates fractions of normal, Alzheimer's, and Parkinson's samples in which TLR14 was detected.

### Example 3

### Generation and Characterization of Antibodies Against TLR14

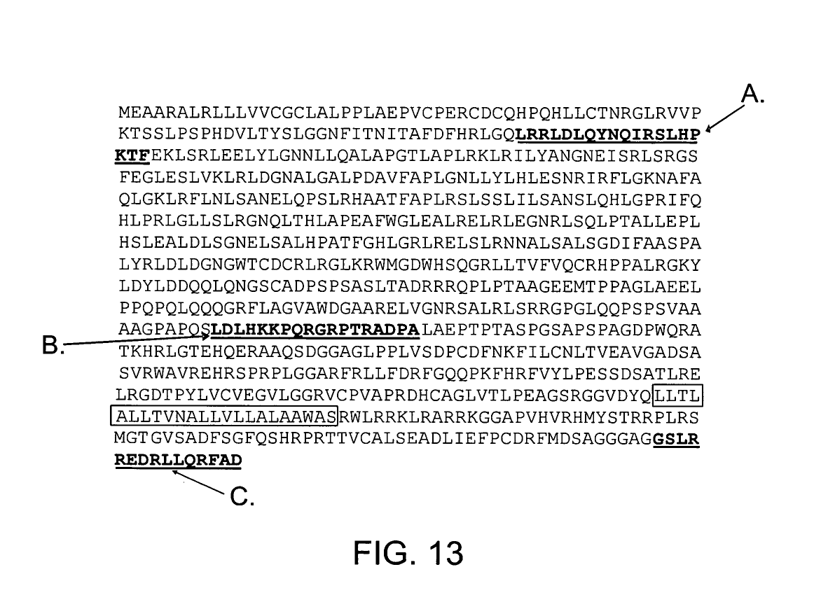

Three peptide purified polyclonal rabbit antibodies were generated against TLR14. Two of the antibodies recognize peptides in the ectodomain (0541 and 0540) and one recognizes an intracellular region of the protein (0547) (FIG. 13). 0547 antibody is 100% conserved among several species including human, mouse, and rat. The 0547 antibody was used to identify expression of TLR14 in primary rat astrocyte culture, embryonic day 14 rat cortical neuron, postnatal day 4 rat cerebellar granule neurons (CGN), and postnatal day 7 rat cerebellum lysate. The expected size of TLR14 protein is 88 Kd. TLR14 specific bands were competed with 0547 peptide used to generate the antibody; one band was ˜88 Kd in size and several other bands were smaller suggesting the existence of lower MW species of the protein.

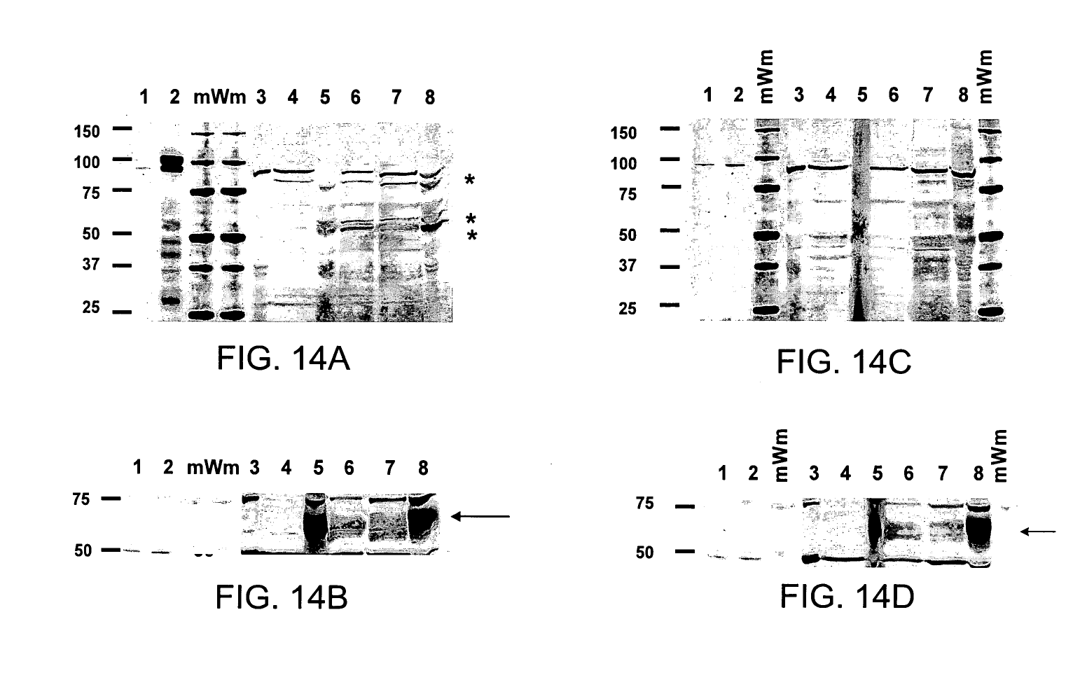

FIGS. 14A-14D depict immunoblots showing anti-TLR14 0547 antibody peptide competition assays. FIGS. 14A and 14B show TLR14 and NgR1 immunoblots performed using 1.8 μg/mL 0547 and NgR1 antibody (R&D systems, goat anti-mouse NgR1 antibody), respectively, in the absence of a competing peptide. FIGS. 14C and 14D shown TLR14 and NgR1 immunoblots performed using 1.8 ug/mL 0547 and NgR1 antibody (R&D systems, goat anti-mouse NgR1 antibody), respectively, in the presence of the 0547 competing peptide. Arrows indicate the band corresponding to NgR.

Comparable amounts of samples from 1) & 3) CHO-K1 cells, 2) CHO-K1 cells over-expressing TLR14, 4) primary rat astrocyte culture, 5) embryonic day14 rat cortical neuron 2-day in vitro culture, 6) postnatal day4 rat cerebellar granule neuron 2-day in vitro culture, 7) postnatal day 4 rat cerebellar granule neuron 1-day in vitro culture, and 8) postnatal day7 rat cerebellum lysate were subjected to Western blots and detected by a TLR14 antibody (0547/2434) (FIG. 14A) with or without (FIG. 14C) pre-incubation with its blocking peptide. TLR14 antibody detected two bands at about 100 kDa in TLR14/CHO-K1 cells. The upper band is specific to this TLR14 antibody since it is competed out when the antibody is pre-treated with its blocking peptide. The lower band at about 100 kDa in TLR14/CHO-K1 and a band in similar location in other samples are not affected by TLR14 blocking peptide suggesting they might not be specific to this TLR14 antibody. Blots were also incubated with NgR1 antibody (FIGS. 14B and 14D) and NgR1 was strongly detected in cortical neuron culture and in cerebellum, and less in cerebellar granule neuron cultures. * indicates bands displaying different patterns after peptide treatment.

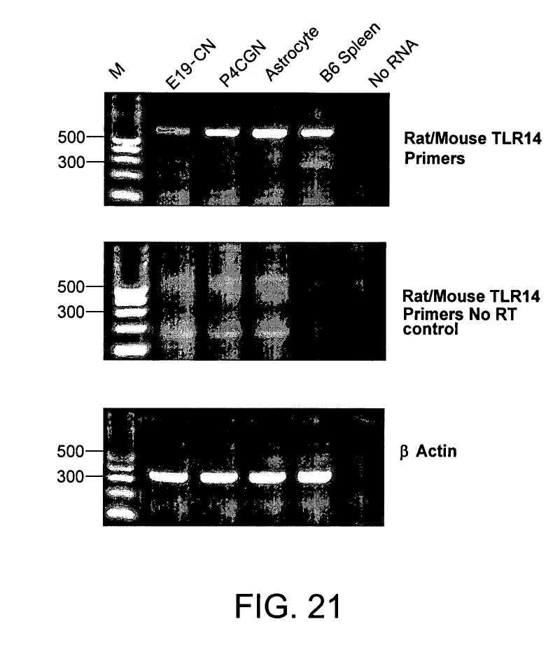

RT-PCR data using primers that can amplify both murine and rat TLR14 mRNA confirmed that TLR14 is expressed in cortical neurons, cerebellar granule neurons and astrocytes (FIG. 21). Briefly, RT PCR on rat and murine TLR14 was performed with Qiagen OneStep RT-PCR kit (Qiagen) following manufacturer's instructions. Around 0.2-0.4 μg of RNA was used and reactions without reverse transcriptase enzyme were used as negative controls. The rat and murine TLR14 primers used were 5′-AGCAGGCAGTAGGCAG-3′ (SEQ ID NO:5) and 5′-GGAACTTGGGCTGCT-3′ (SEQ ID NO:6). b actin RT-PCR control primers (obtained from Invitrogen) were positive controls. The lanes correspond to the following: E19-CN rat primary cells, P4-CGN rat cerebellar granule neurons, and rat primary rat astrocytes. C57B1/6 spleen served as a positive control.

### Example 4

### Expression of TLR14 Protein in Murine Brains

Using two antibodies (0547 and 0541), TLR14 expression was observed in specific regions of the brain in wild type mice.

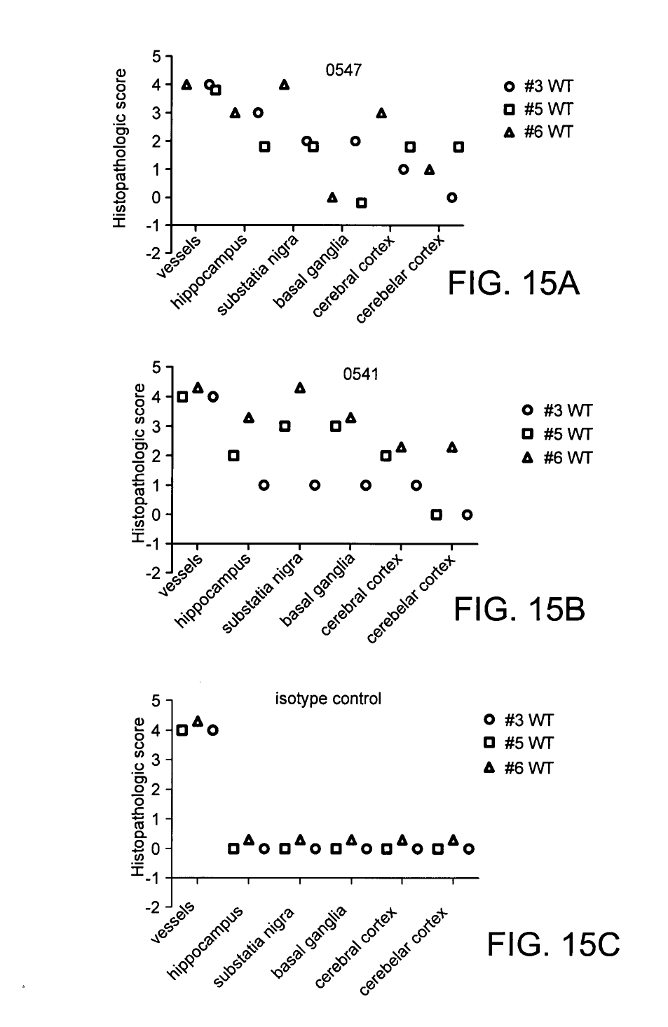

FIG. 15A-15C are scatter plots showing TLR14 expression in the brains of wild type (wt) mice. Three mice were analyzed (wt3-wt6). FIG. 15A shows data generated using the 0547 anti-TLR14 antibody. FIG. 15B shows data generated using the 0541 anti-TLR14 antibody. FIG. 15C shows data generated using an isotypic control antibody.

Staining by immunohistochemistry of 4% paraformaldehyde murine brain sections revealed TLR14 expression in hippocampus, substantia nigra, basal ganglia, cerebral cortex, and the cerebelar cortex and not in any other region of the brain. Both anti-TLR14 antibodies produced the same staining pattern and an isotype control antibody did not stain these regions. In agreement, analysis of Allen Brain Atlas, data show that expression of TLR14 is most prominent in the cortex, hippocampus, and cerebellum Purkinje cell layer (data not shown). The observed localized expression pattern suggests that TLR14 plays a role in these areas of the brain.

### Example 6

### TLR14 Co-Immunoprecipitation Studies

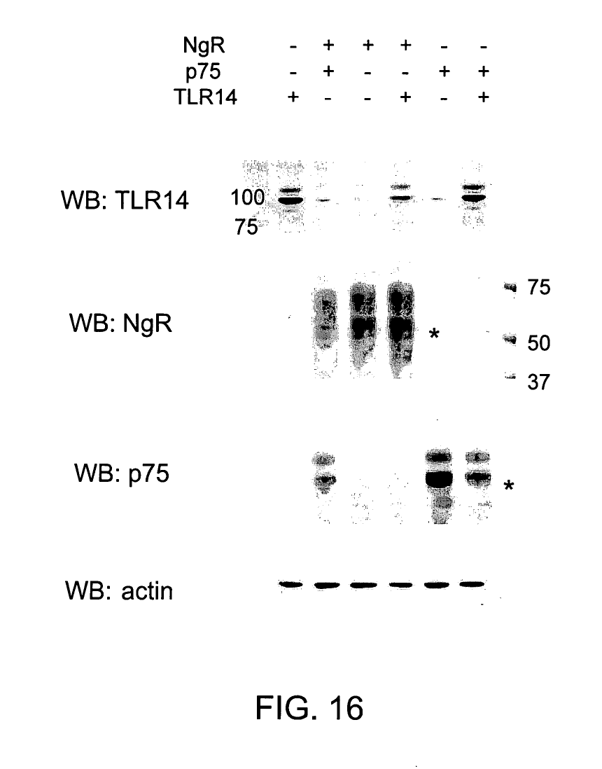

Since TLR14 shares a high sequence homology with NgR1, it was tested whether TLR14 can interact with NgR1 or p75^{NTR} in immunoprecipitation experiments of co-expressed proteins. TLR14 was expressed alone or in combinations with NgR1 and p75 (FIG. 16).

FIG. 16 is an immunoblot showing TLR14 (top), NgR1 (second), p75 (third), and actin (bottom) expression. Star indicates bands corresponding to NgR1 and p75. Actin was used as a loading control. NgR1, p75NTR, or TLR14 construct was expressed alone or in combinations in CHO-K1 cells. Lysates were western blotted with an antibody to TLR14, NgR1 (R&D systems, goat anti-mouse NgR1 antibody), p75NTR (Promega, rabbit anti-human p75 antibody), or actin (Sigma) as indicated. Detection of each protein by its own antibody confirmed expression in the cells. Loading was equal as indicated by similar expression of actin in each samples.

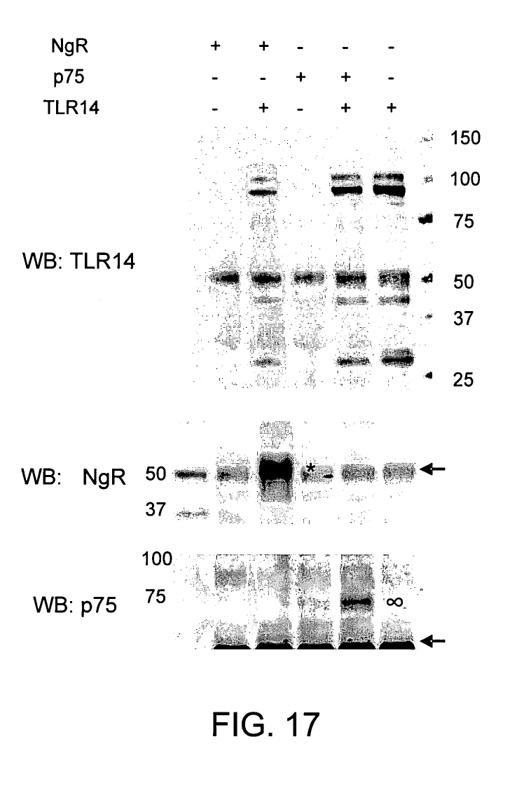

Following immunoprecipitation of TLR14 with the 0547 antibody, both NgR1 and p75NTR were observed to interact with TLR14 (FIG. 17). FIG. 17 depicts an immunoblot showing TLR14, NgR1, and p75NTR following immunoprecipitation using 2 ug of TLR14 0547 antibody. Immunoprecipitations were performed using 0.5 mg cell lysate. Star indicates a band corresponding to NgR1. Epsilon indicates a band corresponding to p75NTR. Arrow indicates the immunoglobulin light chain of antibody. Molecular weight markers are shown on the right of the immunoblots. NgR1, p75NTR, or TLR14 construct was expressed alone or in combinations in CHO-K1 cells. Immunoprecipitates by the TLR14 antibody (0547/2434) have been analyzed by Western blots with the same TLR14 antibody, or with an antibody to NgR1 or p75NTR as indicated. TLR14 was immunoprecipitated by its antibody when expressed in the cells. The lower band of NgR1 or p75NTR was also co-precipitated with TLR14.

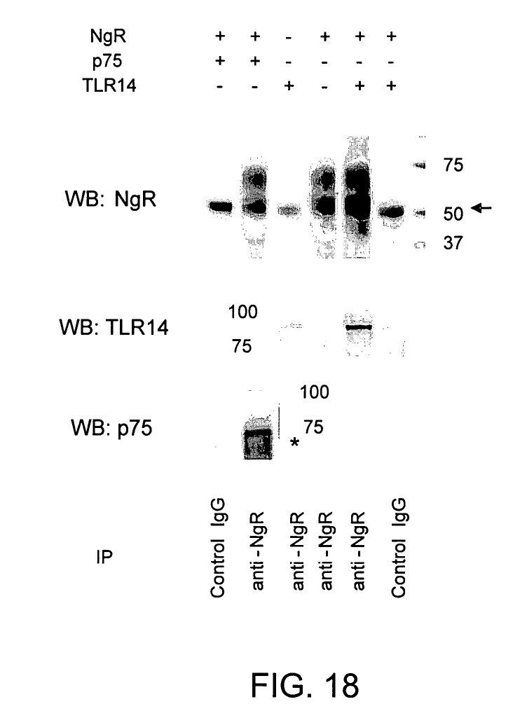

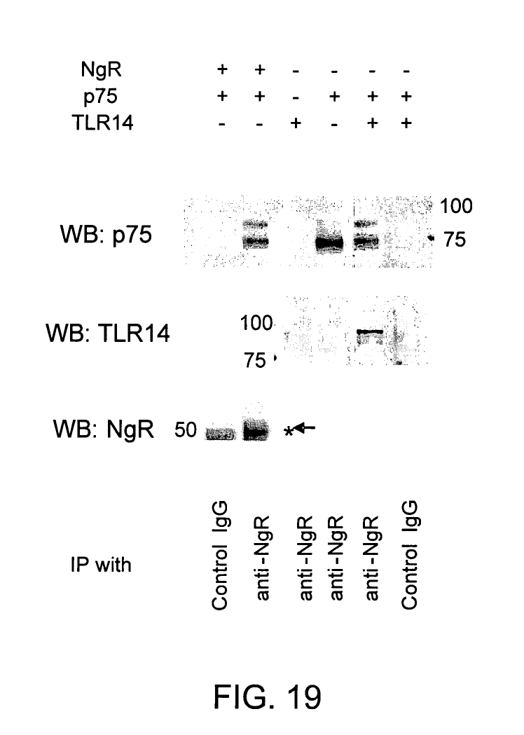

Similarly, immunoprecipitation with anti-NgR1 antibody (FIG. 18) or anti-p75 antibody (FIG. 19) revealed an interaction between TLR14 and NgR1 or TLR14 and p75 when the proteins were co-expressed.

More specifically, FIG. 18 is an immunoblot showing NgR1, TLR14, p75NTR following immunoprecipitation using anti-NgR1 antibody (R&D system, goat anti-human NgR1 antibody) (note this antibody is different from the one used for Western immunoblot above). Star indicates a band corresponding to p75NTR. Arrow indicates the immunoglobulin light chain of antibody. NgR1, p75NTR, or TLR14 construct was expressed alone or in combinations in CHO-K1 cells. Immunoprecipitates by the NgR1 or a control goat IgG antibody have been analyzed by Western blot with NgR1 antibody, or with an antibody to TLR14 or p75NTR as indicated. NgR1 was immunoprecipitated by its antibody when expressed in the cells. The lower band of TLR14 or p75NTR was also co-precipitated with NgR1. Control goat IgG was not able to precipitate NgR1.

FIG. 19 depicts an immunoblot showing NgR1, TLR14, p75NTR following immunoprecipitation using anti-p75NTR antibody. Star indicates a band corresponding to NgR1. Arrow indicates the immunoglobulin light chain of antibody. An NgR1, p75NTR, or TLR14 construct was expressed alone or in various combinations in CHO-K1 cells. Immunoprecipitates by the p75NTR or a control rabbit IgG antibody have been analyzed by Western blot with p75NTR antibody, or with an antibody to TLR14 or NgR1 as indicated. p75NTR was immunoprecipitated by its antibody when expressed in the cells. The lower band of TLR14 or NgR1 was also co-precipitated with p75NTR. Control rabbit IgG was not able to precipitate p75NTR.

### Example 7

### Antibodies Against the Extracellular Domain of TLR14 Reverse Myelin Inhibition of Neurite Outgrowth

To test the function of TLR14, postnatal day 4 rat cerebellar granule neurons were cultured over rat myelin substrate in the presence or absence of Y (10 uM), or the three different purified TLR14 antibodies: 0540, 0541, 0547.

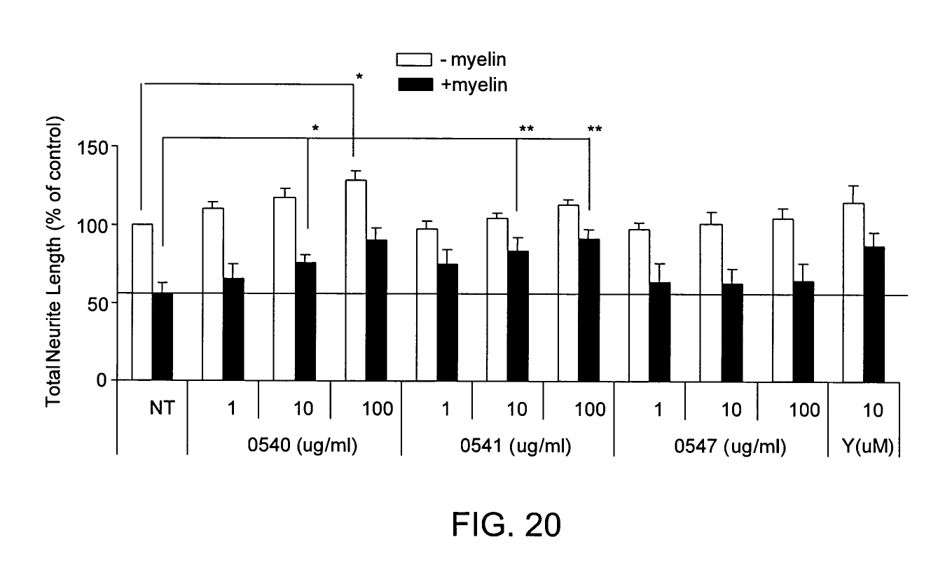

FIG. 20 is a bar graph showing the effect of purified TLR14 antibodies: 0540, 0541, 0547 in neurite outgrowth of P4 CGN (cerebellar granule neuron) cells. NT indicates control cells with no treatment. Y-27632 is a propriety compound known to induce neurite outgrowth in the presence of myelin. (* p=,0.05; ** p=,0.01). Postnatal day4 rat cerebellar granule neurons were cultured over rat myelin substrate in the presence or absence of Y-27632 (10 uM), or 3 different purified TLR14 antibodies: 0540, 0541, 0547 at 1, 10 or 100 ug/ml as indicated. TLR14 antibodies 0540 and 0541 reverse myelin inhibition in a dose-dependent manner. In addition, 0540 at 100 ug/ml promotes basal neurite outgrowth significantly. The cultures were maintained for 22 hrs before being fixed and stained for neuronal class III β-tubulin (rabbit polyclonal Tuj1, Covance). Neuron images were captured by ImageXpress Micro and quantitated by MetaXpress's neurite outgrowth module. Mean total neurite length was determined from more than 200 neurons in triplicate under each culture condition. The results are pooled from three independent experiments and the bars show the SEM.

Extracellular TLR14 antibodies 0540 and 0541 reverse myelin inhibition of neurite outgrowth in a dose-dependent manner. In addition, 0540 at 100 ug/ml promotes basal neurite outgrowth significantly. The internal 0547 antibody does not reverse myelin inhibition of neurite outgrowth. These data suggest that the antibodies, by binding surface TLR14 are able to block the interaction of myelin ligands and block the myelin mediated neurite outgrowth inhibition. This effect may be through modulation of the NgR/p75^{NTR} complex.

All together, these results implicate TLR14 in playing a role in disorders associated with brain injury and suggest that TLR14 can be a useful therapeutic target for enhancing neuronal growth.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of reducing one or more biological activities of Toll like receptor 14 (TLR14) in a neural cell or tissue, comprising:
contacting the neural cell or tissue with an antagonist of TLR14 activity or expression, in an amount sufficient to reduce one or more biological activities of TLR14 in the cell or tissue.

2. The method of claim 1, wherein the TLR14 antagonist is selected from the group consisting of an anti-TLR14 antibody molecule, a soluble form of TLR14, and a nucleic acid molecule that inhibits TLR14 expression.

3. The method of claim 1, wherein the neural cell or tissue comprises one or more cells chosen from dopaminergic, cholinergic, cortical, cerebellar, hippocampal, spinal cord neuronal cells, microglia, astrocytes or oligodendrocytes.

4. The method of claim 1, wherein the contacting step occurs in a cell lysate, a reconstituted system, cells in culture, or cells present in a subject.

5. (canceled)

6. The method of claim 4, wherein the subject is a human patient having, or at risk of having, a neurodegenerative disorder or condition.

7. The method of claim 1, wherein the antagonist reduces one or more of:
(i) modulation of NgR1 binding or activity;
(ii) modulation of p75^{NTR} binding or activity;
(iii) modulation of one or more endotoxin-mediated inflammatory signals in the brain; or (iv) modulation of one or more of: neuronal cell survival, differentiation, neurite outgrowth and/or axonal regeneration.

8. (canceled)

9. (canceled)

10. The method of claim 2, wherein the TLR14 antagonist is an antibody molecule that binds to human TLR14.

11. The method of claim 10, wherein the antibody molecule is a human, humanized, chimeric, camelid, or in vitro generated antibody to human TLR14.

12. The method of claim 11, wherein the antibody molecule comprises two complete heavy chains, and two complete light chains, or an antigen-binding fragment thereof.

13. The method of claim 10, wherein the TLR14 antagonist binds to the extracellular domain of human TLR14.

14. The method of claim 13, wherein the TLR14 antagonist binds to an epitope on TLR14 comprising about amino acids 85 to 103, or 509 to 526 of SEQ ID NO:2.

15. The method of claim 1, wherein the TLR14 antagonist inhibits the expression of nucleic acid encoding TLR14.

16. The method of claim 15, wherein the TLR14 antagonist is selected from the group consisting of an antisense molecule, a ribozyme, RNAi, and a triple helix molecule.

17. The method of claim 6, wherein the neurodegenerative disorder or condition is chosen from one or more of: Parkinson's disease, Alzheimer's disease, Huntington's disease, amyotrophic lateral sclerosis (ALS), traumatic brain injury, spinal cord injury, multiple sclerosis, ischemia or ischemia-induced injury, stroke, or a neurodegenerative condition or disorder caused by a bacterial infection.

18. (canceled)

19. A method of treating or preventing a neurodegenerative disorder or condition in a subject, comprising:
administering to the subject a TLR14 antagonist, in an amount sufficient to inhibit or reduce one or more TLR14 biological activities in a neural cell or tissue, thereby treating the disorder.

20. The method of claim 19, wherein the TLR14 antagonist is selected from the group consisting of an anti-TLR14 antibody molecule, a soluble form of TLR14, and a nucleic acid molecule that inhibits TLR14 expression.

21. The method of claim 19, wherein the neurodegenerative disorder or condition is associated with disorder chosen from one or more of: Parkinson's disease, Alzheimer's disease, Huntington's disease, amyotrophic lateral sclerosis (ALS), traumatic brain injury, spinal cord injury, multiple sclerosis, ischemia or ischemia-induced injury, stroke, or a neurodegenerative condition or disorder caused by a bacterial infections.

22. The method of claim 19, wherein the TLR14 antagonist binds to the extracellular domain of human TLR14.

23. A method for stimulating one or more of neurite outgrowth, survival, or differentiation of a neuronal cell chosen from a dopaminergic, cholinergic, cortical, cerebellar, hippocampal or spinal cord neuronal cell, the method comprising contacting the cell with an antibody molecule that interacts with the extracellular domain of TLR14, thereby stimulating one or more of neurite outgrowth, survival, or differentiation of the neuronal cell.

24. A method for regenerating or repairing neural tissue in a subject, comprising administering a TLR14 antagonist to the subject in an amount sufficient to reduce the association of activity or expression of TLR14 in the subject.

25. A method of reducing or inhibiting an immune response associated with a neurodegenerative disease, in a subject, the method comprising administering to the subject a TLR14 antagonist in an amount sufficient to decrease the association or activity of TLR14 and LPS in the CNS, thereby reducing or inhibiting the immune response.

26. A method of evaluating, diagnosing, or monitoring the progression of, a TLR14 associated neurodegenerative disorder in a test sample, comprising:
evaluating the expression or activity of a TLR14 nucleic acid or polypeptide, such that, a difference in the level of the nucleic acid or polypeptide relative to a reference sample is indicative of the presence or progression of the neurodegenerative disorder.

27. A method, or an assay, for identifying a compound that modulates neural function, comprising:
providing or identifying a test compound that interacts binds to TLR14 or a TLR14-associated polypeptide or nucleic acid, wherein the TLR14-associated polypeptide or nucleic acid is a NgR1 or a p75^{NTR} polypeptide or nucleic acid;
contacting one or more of: a TLR14 or TLR14 receptor polypeptide, or a nucleic acid encoding the TLR14 or TLR14 receptor, with the test compound; and
detecting a change in one or more activities of the TLR14 or the TLR14 receptor polypeptide or nucleic acid, in the presence of the test compound, relative to a control sample without the test compound.

28. The method, or assay, of claim 27, wherein the test compound is selected from the group consisting of an antibody molecule, TLR14 peptide, a small molecule, a naturally-occurring TLR14 antagonist, an antisense molecule, a ribozyme, an RNAi and a triple helix molecule.

29. (canceled)

30. The method of claim 27, wherein the change in an activity of the neural cell or tissue is detected by measuring a change, in the presence of the test compound, relative to a reference sample in one or more of:
(i) modulation of NgR1 binding or activity;
(ii) modulation of p75^{NTR} binding or activity;
(iii) modulation of one or more endotoxin-mediated inflammatory signals in the brain; or
(iv) modulation of one or more of: neuronal cell survival, differentiation, neurite outgrowth, or axonal regeneration.

31. (canceled)

32. An antibody molecule that binds to the extracellular domain of human TLR14.

33. The antibody molecule of claim 32 that binds to an epitope on TLR14 comprising about amino acids 85 to 103, or 509 to 526 of SEQ ID NO:2.

34. A pharmaceutical composition comprising the antibody molecule according to claim 32.


SEQUENCE LISTING (16 sequences)

SEQ ID NO: 1 - DNA, 2436 bp, Homo sapiens
atggaggctgcccgcgccttgcgcctcctgctcgtggtgtgcggctgcctcgcgctcccgccgctggccgagcccgtgtgcccggagcgctgcgactgccagcatccccagcatctcctgtgcaccaacagggggctccgcgtagtgcccaagaccagctcgctgccgagcccccacgacgtgctcacctacagcctcggcggcaacttcataaccaacatcacggccttcgacttccaccgtctggggcagctcagacggctggacctgcagtacaaccagatccgctctctgcaccccaagaccttcgagaagctctcgcggctggaagagctgtacctggggaacaacctcttgcaggcgctcgccccgggcacgctggccccgctgcgcaagctgcgcatcctctacgccaacgggaacgagatcagccgcctaagccgcggctccttcgagggcctggagagtctagtcaagctgcggctggacgggaacgccctgggggcgctgccggacgcggtcttcgctcccttgggcaacctgctctacctacatctggagtccaaccggatccgctttctgggcaagaacgccttcgcccagctaggcaagctgcgcttcctcaacctctctgccaacgagctacagccctccctgcgccacgcggccaccttcgcaccgctgcgctccctctcctccctcatcctctcggccaacagcctgcagcacctcgggccgcgcatcttccagcacctgccacgtctcggcctgctctcgctcaggggcaaccagctcacgcacctcgcgcctgaggccttttggggcttggaggccctgcgcgagctgcgcctggagggtaatcggctgagccagctgccaactgcgctgctggagcctctgcacagcctggaggcgctggacctgagcggcaatgagctgtccgccctgcacccggccaccttcggccacctgggccggctgcgcgagctcagcctgcgcaacaacgcgctcagcgccctatccggggacatcttcgccgccagcccagccctttatcggctggatctagacggcaacggctggacctgcgactgccggctgcgaggcctgaagcgctggatgggcgactggcactcgcagggccggctcctcactgtcttcgtgcagtgtcgccaccccccggccctgcgaggcaaatacctggattacctggatgaccagcagctgcaaaatggatcctgcgcggatccctcgccctcagcttccctgaccgctgaccgcaggcggcagcccctacccacggccgcaggggaggagatgacgccacctgcaggtctcgcggaggagctgccgccgcagccgcagctccagcagcaggggcgatttctagctggggtggcctgggatggggccgccagggagctggtaggcaaccgcagcgctctaaggctgagtcggcggggcccgggcctccagcagcccagcccctccgtcgctgccgccgcgggcccggctccacagtccctagacctgcacaagaagccccagcggggccgtccgactcgggcagatcccgccctcgcggagcccaccccaacggcctctcctggctctgcgccatcgcccgccggcgacccctggcagcgcgcgacgaagcatcgtctgggcacggagcaccaggagcgtgccgcccagtccgacggtggggccgggctgccgccgctggtgtccgacccatgcgacttcaacaagttcattctgtgcaacctgacggtggaggcggtgggcgcagacagcgcctcggtgcgctgggccgtgcgcgagcaccgcagtccccggccgctgggcggcgcgcgcttccgcctgctctttgaccgctttggccagcagcccaagttccaccgcttcgtctacctgcctgagagcagcgactcggccacgctgcgcgagctgcgcggggacaccccctacctggtgtgcgtggagggcgtgcttgggggccgtgtctgccctgtggctccccgggaccactgcgcggggctggtcaccctaccggaggccgggagccggggcggcgtcgactaccagctgctgaccttggccctgctgacggtcaacgcgctgctggtgctcctggccttggcggcctgggcgtctcgctggctgcgtaggaaactgcgggctaggcggaagggcggggccccggtccacgttcggcacatgtactccacccgacggcccctgcgctccatgggcaccggcgtgtccgccgacttctcgggattccagtcgcaccggccacgcaccaccgtgtgcgcgctcagtgaggcggacctcatcgaattcccctgcgaccgcttcatggacagtgcgggcggcggcgcgggcggcagcctgagacgggaggaccgtctcctgcagcgatttgccgactag

SEQ ID NO: 2 - protein, 811 aa, Homo sapiens
MEAARALRLLLVVCGCLALPPLAEPVCPERCDCQHPQHLLCTNRGLRVVPKTSSLPSPHDVLTYSLGGNFITNITAFDFHRLGQLRRLDLQYNQIRSLHPKTFEKLSRLEELYLGNNLLQALAPGTLAPLRKLRILYANGNEISRLSRGSFEGLESLVKLRLDGNALGALPDAVFAPLGNLLYLHLESNRIRFLGKNAFAQLGKLRFLNLSANELQPSLRHAATFAPLRSLSSLILSANSLQHLGPRIFQHLPRLGLLSLRGNQLTHLAPEAFWGLEALRELRLEGNRLSQLPTALLEPLHSLEALDLSGNELSALHPATFGHLGRLRELSLRNNALSALSGDIFAASPALYRLDLDGNGWTCDCRLRGLKRWMGDWHSQGRLLTVFVQCRHPPALRGKYLDYLDDQQLQNGSCADPSPSASLTADRRRQPLPTAAGEEMTPPAGLAEELPPQPQLQQQGRFLAGVAWDGAARELVGNRSALRLSRRGPGLQQPSPSVAAAAGPAPQSLDLHKKPQRGRPTRADPALAEPTPTASPGSAPSPAGDPWQRATKHRLGTEHQERAAQSDGGAGLPPLVSDPCDFNKFILCNLTVEAVGADSASVRWAVREHRSPRPLGGARFRLLFDRFGQQPKFHRFVYLPESSDSATLRELRGDTPYLVCVEGVLGGRVCPVAPRDHCAGLVTLPEAGSRGGVDYQLLTLALLTVNALLVLLALAAWASRWLRRKLRARRKGGAPVHVRHMYSTRRPLRSMGTGVSADFSGFQSHRPRTTVCALSEADLIEFPCDRFMDSAGGGAGGSLRREDRLLQRFAD

SEQ ID NO: 3 - DNA, 2430 bp, Mus musculus
atggagggtgtgggtgccgtgcgcttctggctcgtggtgtgcggctgcctggcgttcccgccgcgggccgagtccgtgtgcccggagcgctgcgactgccagcacccccaacatctcctatgcaccaacaggggacttcgcgctgtacccaagaccagctcgctgcctagtccccaggacgtgctcacctacagcctaggcggcaacttcatcaccaacatcaccgccttcgacttccaccgtctggggcagctcagacggctggatctgcagtacaaccagatccgttctctccaccccaagaccttcgagaagctctcacgactagaggagctgtacctggggaacaacctcttgcaggcgctcgttcctggcacgttggctccgctgcgcaagttgcgcatcctttacgccaacggcaacgagattggacgcctgagtcgcgggtccttcgagggcctggagagtttggtcaagctacggctggacgggaacgtgctgggagcgctgccggacgcggtcttcgctcccctgggcaacctgctctacctacacctggaatctaaccggatccgttttttgggcaagaacgccttcagccagctgggtaagcttcgattcctcaatctctcggccaacgagctgcaaccctctctgcgccacgcggccacctttgtccctctgcgctccctctctactctcatcctctcagccaacagcctacagcacctcggcccccgcgtcttccagcacctgccacgcctaggcctgctctccctcagtggaaaccagctcacgcacctcgccccagaggccttttggggactggaggccctacgtgagctgcgcctggaaggtaaccggctgaaccagctccccctgacactgctggagcctctgcacagcttggaggcactagacctgagtggcaatgagctttctgctctgcaccctgccacctttggccaccagggccggctccgtgagctcagcctgcgcgacaacgcgctcagcgccctttctggggacatctttgccgccagtccggctctctaccggctggatctagacggcaatggttggacctgcgactgccggttgcggggtctgaaacgctggatgggcaactggcattctcagggtcgcctccttacggtcttcgtgcagtgtcgccaccccccggccctgcggggcaagtacctggattacctggatgaccaactgctgcagaatgggtcttgcgtggatccctcaccttcccctacagcaggcagtaggcagtggccgctacccacatcgtcagaagaggggatgacgccccctgcaggcctctcgcaggagctaccactgcagccgcagccgcagccccagcagcggggaagacttctaccaggagtggcctggggtggggctgccaaggaactggtgggcaaccgcagcgcattaaggttgagccggcggggaccaggtccccatcagggcccctctgctgcggctccgggctctgccccacagtccttggacctgcacgagaagccggggagaggacgtcacacccgagccaatctctcccagacggagcccaccccgacgtctgagcccgcctctgggacaccatctgccagagacagctggcagcgcgcagcaaagcagcgcctagcgtccgagcagcaagagagcgcagtccagtccgttagtggggttggtttgccacctctggtgtctgacccctgtgacttcaacaaattcattctgtgcaacctgacagtagaggcggtgagcgccaacagcgcctcggtgcgctgggcggttcgcgaacaccgcagtccccggccgcagggcggcgcacgcttccgcctgcttttcgaccgctttggccagcagcccaagttccagcgcttcgtctacctgccggagcgcagcgactcggccacgctgcacgagctgcgcggagacaccccctacctagtatgcgtggagggtgtgctcgggggtcgcgtttgccccgtggccccccgagaccactgcgcagggttagtaaccctgccagaggcaggaggtcgaggtggcgtcgactaccagctcctgaccttggtcttgctggctgtcaacgcgcttctggtgctcctggcactggcagcctggggatcgcgatggctgcggaggaagctgcgtgccaggcggaagggaggggccccggtccacgttcgccacatgtactccacccgacggccactgcgctccatgggcactggtgtgtccgcggacttctcaggcttccagtcgcaccgtccccgcaccaccgtgtgcgcgctcagcgaggccgacctcattgagttcccctgcgaccgcttcatggacagcaccggcggtggtaccagtggcagcttgaggcgggaggatcacctcttgcaaagattcgctgactag

SEQ ID NO: 4 - protein, 809 aa, Mus musculus
MEGVGAVRFWLVVCGCLAFPPRAESVCPERCDCQHPQHLLCTNRGLRAVPKTSSLPSPQDVLTYSLGGNFITNITAFDFHRLGQLRRLDLQYNQIRSLHPKTFEKLSRLEELYLGNNLLQALVPGTLAPLRKLRILYANGNEIGRLSRGSFEGLESLVKLRLDGNVLGALPDAVFAPLGNLLYLHLESNRIRFLGKNAFSQLGKLRFLNLSANELQPSLRHAATFVPLRSLSTLILSANSLQHLGPRVFQHLPRLGLLSLSGNQLTHLAPEAFWGLEALRELRLEGNRLNQLPLTLLEPLHSLEALDLSGNELSALHPATFGHQGRLRELSLRDNALSALSGDIFAASPALYRLDLDGNGWTCDCRLRGLKRWMGNWHSQGRLLTVFVQCRHPPALRGKYLDYLDDQLLQNGSCVDPSPSPTAGSRQWPLPTSSEEGMTPPAGLSQELPLQPQPQPQQRGRLLPGVAWGGAAKELVGNRSALRLSRRGPGPHQGPSAAAPGSAPQSLDLHEKPGRGRHTRANLSQTEPTPTSEPASGTPSARDSWQRAAKQRLASEQQESAVQSVSGVGLPPLVSDPCDFNKFILCNLTVEAVSANSASVRWAVREHRSPRPQGGARFRLLFDRFGQQPKFQRFVYLPERSDSATLHELRGDTPYLVCVEGVLGGRVCPVAPRDHCAGLVTLPEAGGRGGVDYQLLTLVLLAVNALLVLLALAAWGSRWLRRKLRARRKGGAPVHVRHMYSTRRPLRSMGTGVSADFSGFQSHRPRTTVCALSEADLIEFPCDRFMDSTGGGTSGSLRREDHLLQRFAD

SEQ ID NO: 5 - DNA, 16 bp, Artificial Sequence
  Description of Artificial Sequence Synthetic primer
agcaggcagtaggcag

SEQ ID NO: 6 - DNA, 15 bp, Artificial Sequence
  Description of Artificial Sequence Synthetic primer
ggaacttgggctgct

SEQ ID NO: 7 - DNA, 1422 bp, Homo sapiens
atgaagagggcgtccgctggagggagccggctgctggcatgggtgctgtggctgcaggcctggcaggtggcagccccatgcccaggtgcctgcgtatgctacaatgagcccaaggtgacgacaagctgcccccagcagggcctgcaggctgtgcccgtgggcatccctgctgccagccagcgcatcttcctgcacggcaaccgcatctcgcatgtgccagctgccagcttccgtgcctgccgcaacctcaccatcctgtggctgcactcgaatgtgctggcccgaattgatgcggctgccttcactggcctggccctcctggagcagctggacctcagcgataatgcacagctccggtctgtggaccctgccacattccacggcctgggccgcctacacacgctgcacctggaccgctgcggcctgcaggagctgggcccggggctgttccgcggcctggctgccctgcagtacctctacctgcaggacaacgcgctgcaggcactgcctgatgacaccttccgcgacctgggcaacctcacacacctcttcctgcacggcaaccgcatctccagcgtgcccgagcgcgccttccgtgggctgcacagcctcgaccgtctcctactgcaccagaaccgcgtggcccatgtgcacccgcatgccttccgtgaccttggccgcctcatgacactctatctgtttgccaacaatctatcagcgctgcccactgaggccctggcccccctgcgtgccctgcagtacctgaggctcaacgacaacccctgggtgtgtgactgccgggcacgcccactctgggcctggctgcagaagttccgcggctcctcctccgaggtgccctgcagcctcccgcaacgcctggctggccgtgacctcaaacgcctagctgccaatgacctgcagggctgcgctgtggccaccggcccttaccatcccatctggaccggcagggccaccgatgaggagccgctggggcttcccaagtgctgccagccagatgccgctgacaaggcctcagtactggagcctggaagaccagcttcggcaggcaatgcgctgaagggacgcgtgccgcccggtgacagcccgccgggcaacggctctggcccacggcacatcaatgactcaccctttgggactctgcctggctctgctgagcccccgctcactgcagtgcggcccgagggctccgagccaccagggttccccacctcgggccctcgccggaggccaggctgttcacgcaagaaccgcacccgcagccactgccgtctgggccaggcaggcagcgggggtggcgggactggtgactcagaaggctcaggtgccctacccagcctcacctgcagcctcacccccctgggcctggcgctggtgctgtggacagtgcttgggccctgctga

SEQ ID NO: 8 - protein, 473 aa, Homo sapiens
MKRASAGGSRLLAWVLWLQAWQVAAPCPGACVCYNEPKVTTSCPQQGLQAVPVGIPAASQRIFLHGNRISHVPAASFRACRNLTILWLHSNVLARIDAAAFTGLALLEQLDLSDNAQLRSVDPATFHGLGRLHTLHLDRCGLQELGPGLFRGLAALQYLYLQDNALQALPDDTFRDLGNLTHLFLHGNRISSVPERAFRGLHSLDRLLLHQNRVAHVHPHAFRDLGRLMTLYLFANNLSALPTEALAPLRALQYLRLNDNPWVCDCRARPLWAWLQKFRGSSSEVPCSLPQRLAGRDLKRLAANDLQGCAVATGPYHPIWTGRATDEEPLGLPKCCQPDAADKASVLEPGRPASAGNALKGRVPPGDSPPGNGSGPRHINDSPFGTLPGSAEPPLTAVRPEGSEPPGFPTSGPRRRPGCSRKNRTRSHCRLGQAGSGGGGTGDSEGSGALPSLTCSLTPLGLALVLWTVLGPC

SEQ ID NO: 9 - DNA, 1890 bp, Mus musculus
ccgcagcccgcgagcccagcccggcccggtagagcggagcgccggagcctcgtcccgcggccgggccgggaccgggccggagcagcggcgcctggatgcggacccggccgcgcgcagacgggcgcccgccccgaagccgcttccagtgcccgacgcgccccgctcgaccccgaagatgaagagggcgtcctccggaggaagcaggctgctggcatgggtgttatggctacaggcctggagggtagcaacaccatgccctggtgcttgtgtgtgctacaatgagcccaaggtaacaacaagctgcccccagcagggtctgcaggctgtgcccactggcatcccagcctctagccagcgaatcttcctgcatggcaaccgaatctctcacgtgccagctgcgagcttccagtcatgccgaaatctcactatcctgtggctgcactctaatgcgctggctcggatcgatgctgctgccttcactggtctgaccctcctggagcaactagatcttagtgataatgcacagcttcatgtcgtggaccctaccacgttccacggcctgggccacctgcacacactgcacctagaccgatgtggcctgcgggagctgggtcccggcctattccgtggactagcagctctgcagtacctctacctacaagacaacaatctgcaggcactccctgacaacacctttcgagacctgggcaacctcacgcatctctttctgcatggcaaccgtatccccagtgtgcctgagcacgctttccgtggcctgcacagtcttgaccgcctcctcttgcaccagaaccatgtggctcgtgtgcacccacatgccttccgggaccttggccgcctcatgaccctctacctgtttgccaacaacctctccatgctgcctgcagaggtcctaatgcccctgaggtctctgcagtacctgcgactcaatgacaacccctgggtgtgtgactgccgggcacgtccactctgggcctggctgcagaagttccgaggttcctcatcagaggtgccctgcaacctgccccaacgcctggcagaccgtgatcttaagcgcctcgctgccagtgacctagagggctgtgctgtggcttcaggacccttccgtcccatccagaccagtcagctcactgatgaggagctgctgagcctccccaagtgctgccagccagatgctgcagacaaagcctcagtactggaacccgggaggccagcttctgccggaaacgccctcaagggacgtgtgcctcccggtgacactccaccaggcaatggctcaggccctcggcacatcaatgactctccatttggaactttgcccagctctgcagagcccccactgactgccctgcggcctgggggttccgagccaccaggacttcccaccactggtccccgcaggaggccaggttgttcccggaagaatcgcacccgcagccactgccgtctgggccaggcgggaagtggggccagtggaacaggggacgcagagggttcaggggctctgcctgctctggcctgcagccttgctcctctgggccttgcactggtactttggacagtgcttgggccctgctgaccagccaccagccaccaggtgtgtgtacatatggggtctccctccacgccgccagccagagccagggacaggctctgaggggcaggccaggccctccctgacagatgcctccccaccagcccacccccatctccaccccatcatgtttacagggttccgggggtggcgtttgttccagaacgccacctcccacccggatcgcggtatatagagatatgaattttattttacttgtgtaaaatatcggatgacgtggaataaagagctcttttcttaaaaaaaaaaaaaaaaaa

SEQ ID NO: 10 - protein, 473 aa, Mus musculus
PKRASSGGSRLLAWVLWLQAWRVATPCPGACVCYNEPKVTTSCPQQGLQAVPTGIPASSQRIFLHGNRISHVPAASFQSCRNLTILWLHSNALARIDAAAFTGLTLLEQLDLSDNAQLHVVDPTTFHGLGHLHTLHLDRCGLRELGPGLFRGLAALQYLYLQDNNLQALPDNTFRDLGNLTHLFLHGNRIPSVPEHAFRGLHSLDRLLLHQNHVARVHPHAFRDLGRLMTLYLFANNLSMLPAEVLMPLRSLQYLRLNDNPWVCDCRARPLWAWLQKFRGSSSEVPCNLPQRLADRDLKRLAASDLEGCAVASGPFRPIQTSQLTDEELLSLPKCCQPDAADKASVLEPGRPASAGNALKGRVPPGDTPPGNGSGPRHINDSPFGTLPSSAEPPLTALRPGGSEPPGLPTTGPRRRPGCSRKNRTRSHCRLGQAGSGASGTGDAEGSGALPALACSLAPLGLALVLWTVLGPC

SEQ ID NO: 11 - DNA, 3386 bp, Homo sapiens
gccgcggccagctccggcgggcagggggggcgctggagcgcagcgcagcgcagccccatcagtccgcaaagcggaccgagctggaagtcgagcgctgccgcgggaggcgggcgatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtggaacagctgcaagcagaacaagcaaggagccaacagccggccagtgaaccagacgcccccaccagagggagaaaaactccacagcgacagtggcatctccgtggacagccagagcctgcatgaccagcagccccacacgcagacagcctcgggccaggccctcaagggtgacggaggcctctacagcagcctgcccccagccaagcgggaggaggtggagaagcttctcaacggctctgcgggggacacctggcggcacctggcgggcgagctgggctaccagcccgagcacatagactcctttacccatgaggcctgccccgttcgcgccctgcttgcaagctgggccacccaggacagcgccacactggacgccctcctggccgccctgcgccgcatccagcgagccgacctcgtggagagtctgtgcagtgagtccactgccacatccccggtgtgagcccaaccggggagcccccgccccgccccacattccgacaaccgatgctccagccaacccctgtggagcccgcacccccaccctttggggggggcccgcctggcagaactgagctcctctgggcaggacctcagagtccaggccccaaaaccacagccctgtcagtgcagcccgtgtggccccttcacttctgaccacacttcctgtccagagagagaagtgcccctgctgcctccccaaccctgcccctgccccgtcaccatctcaggccacctgcccccttctcccacactgctaggtgggccagcccctcccaccacagcaggtgtcatatatggggggccaacaccagggatggtactagggggaagtgacaaggccccagagactcagagggaggaatcgaggaaccagagccatggactctacactgtgaacttggggaacaagggtggcatcccagtggcctcaaccctccctcagcccctcttgccccccaccccagcctaagatgaagaggatcggaggcttgtcagagctgggaggggttttcgaagctcagcccacccccctcattttggatataggtcagtgaggcccagggagaggccatgattcgcccaaagccagacagcaacggggaggccaagtgcaggctggcaccgccttctctaaatgaggggcctcaggtttgcctgagggcgaggggagggtggcaggtgaccttctgggaaatggcttgaagccaagtcagctttgccttccacgctgtctccagacccccaccccttccccactgcctgcccacccgtggagatgggatgcttgcctagggcctggtccatgatggagtcaggtttggggttcgtggaaagggtgctgcttccctctgcctgtccctctcaggcatgcctgtgtgacatcagtggcatggctccagtctgctgccctccatcccgacatggacccggagctaacactggcccctagaatcagcctaggggtcagggaccaaggacccctcaccttgcaacacacagacacacgcacacacacacacaggaggagaaatctcacttttctccatgagttttttctcttgggctgagactggatactgcccggggcagctgccagagaagcatcggagggaattgaggtctgctcggccgtcttcactcgcccccgggtttggcgggccaaggactgccgaccgaggctggagctggcgtctgtcttcaagggcttacacgtggaggaatgctcccccatcctccccttccctgcaaacatggggttggctgggcccagaaggttgcgatgaagaaaagcgggccagtgtgggaatgcggcaagaaggaattgacttcgactgtgacctgtggggatttctcccagctctagacaaccctgcaaaggactgttttttcctgagcttggccagaagggggccatgaggcctcagtggactttccaccccctccctggcctgttctgttttgcctgaagttggagtgagtgtggctcccctctatttagcatgacaagccccaggcaggctgtgcgctgacaaccaccgctccccagcccagggttcccccagccctgtggaagggactaggagcactgtagtaaatggcaattctttgacctcaacctgtgatgaggggaggaaactcacctgctggcccctcacctgggcacctggggagtgggacagagtctgggtgtatttattttcctccccagcaggtggggagggggtttggtggcttgcaagtatgttttagcatgtgtttggttctggggcccctttttactccccttgagctgagatggaacccttttggcccccagctgggggccatgagctccagacccccagcaaccctcctatcacctcccctccttgcctcctgtgtaatcatttcttgggccctcctgaaacttacacacaaaacgttaagtgatgaacattaaatagcaaag

SEQ ID NO: 12 - protein, 427 aa, Homo sapiens
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSCKQNKQGANSRPVNQTPPPEGEKLHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV

SEQ ID NO: 13 - DNA, 3409 bp, Mus musculus
agagcgagtggagcctccgccagctccggcgggcagcgggcgctggagcgcagcgcagcgcagctcagctcagcgcgcagcaccatcggtccgcagagcggactgagctagaagctgagcgctgtcgccggaggcgagcaatgaggagggcaggtgctgcctgcagcgccatggaccggctgcgcctgctgctgctgctgctgctgcttctaggggtgtcctttggaggtgccaaggagacatgttccacaggcatgtacacccacagtggagagtgctgcaaagcctgcaacctgggcgaaggtgtggcccagccttgcggagccaaccagaccgtgtgtgaaccctgcctggacagtgttacgttctctgacgtggtgagcgccaccgagccgtgcaagccgtgcaccgagtgcctgggcctgcagagtatgtccgctccctgtgtggaggcagacgatgccgtgtgccgatgctcctatggctactaccaggacgaggagactggccgctgcgaggcttgcagcgtgtgcggggtgggctcaggactcgtgttctcctgccaggacaaacagaacacagtgtgtgaagagtgcccagagggcacatactcagatgaagccaaccacgtggacccgtgcctaccctgcacggtgtgcgaggacactgagcgccagttacgcgagtgcacgccctgggctgacgccgaatgcgaggagatccctggccgatggatcacaaggtctacgcccccggagggctctgacgtcacaacacccagcacccaggagccggaggcacctccagagcgagacctcatagccagcacagtggccgatacggtgaccactgtgatgggcagctcccagcctgtagtgacccgaggcaccgctgacaacctcattcctgtctattgctccatcttggctgctgtggttgtgggccttgtggcctatattgctttcaagagatggaacagctgcaagcaaaataaacaaggagccaacagccggccggtgaaccagacacccccaccagagggagagaaactgcacagcgacagcggcatctctgtggacagccagagcctgcacgaccagcagacccacacacagactgcctcaggccaagccctcaagggtgatggcaacctctacagtagcctgcccctgaccaagcgtgaggaggtcgagaagctgctcaatggtgacacctggcgacatctggcaggcgagctgggctaccagccggagcatatagactcctttacccacgaggcctgcccagtccgagccctgctggccagctggggtgcccaggacagcgcgacgctcgatgcccttttagccgccctgcgacgcatccagagagctgacattgtggagagcctgtgcagcgagtccactgccacgtcccctgtgtgagctcaccgactgggagcccctgtcctgtcccacattccgaccgctgatgttctagcccccacagagccgcccccctctcccttggggtggcccaacagtcagaaccgagcatctctgtgtagggcctctgtgtcccgctccccaaaccacagccctgttgctgctcctgaggggacctttgcttctgaccacactctctctccggcgagagataggaccaccccaagcctgtcttgccccgtgaccatctcaggcctttcctttctttctacacattaactgtgtcagatctaggggtctgacactagagaaggtaacaggggcacccctaagactcagggggtactaatgaaccagagccatggactccacactgtgaactggagaacagggggggcattgtggtaggccagaccttcctcaacccctcccttctcccatctggccaaagatgaagaggattacaggcctatctgagctgaaagcaggtttggagcccagtccacactccttctcttacacataggatggtaaagcccagagaggcagggactaacctaggccacccaacaacagggagatcaattgtaggctgacacactcctttctgaatgagggcgtcaagtgcttgttggcagggatggagtgactttcagggaaacatctggaaacgacgtctgccccgccctcaaccacttccaggcccctacccaacccttgtgcagatgaactgtctgttcaaaacctggtccattggcctgttctgatggagtcgggctaatgtcctggggagtggggtgctgccttcctctgtctgtcaggcttagccacagggcatttgtggagggatgaatctgttagcaccctcattcgtgtcataagcctgaagccaacagtggcccttggagtcaaccctaggggtcagggaccaaggacccaccccataatccaacaccacacacacacacacacacacacacacacacagagagagagagagagagagagagagagagagagagagagagagatcatgcttttccccccatggctctttttgggctgagaccagatcctgctggaagtcgctggctgccagagaaagatggaaggggaccgagatgagcttcgtggggctgtcttcctcacctcccggctctggcaggccaagaactgctgcatctgagctggcgtgcgtgcgtccgtccctgtccgtcggtctccaatggcctgtgtgtggaggaatgctcacaccgctccctctcctccccttcttgaagcccaactttggctgcacccagaaaacagtgaagaaaagcaggctgctgtgagaacacaagaaaatgcagatgctggcccgcagtctgaggcaactttctcctcagtttcatggcccctgcaaaggaccaatttcctgggcatggccaggaagggacccgagtgctcattggccggcgctttcacctggctccttggcctgttctgttttgcttgctgttggaatgagtgggctccccctctatttagcatgaaggagccccaggcagggtatgcacagactgactgccatccctccccacccagggtccacccagccctgtaaggagacaaggagccttgtgctcggtggtatttttatggacctcgatctgcaattagtagaaggaccccaccggctggccctccccggacacctgggaagtgggacattctttgtgtatttattttcctcccccagaagctgggggtgggtgggtgggggaggagctgcaggaccagtttagcatgtgtttggttctgggggtctctccagccttattttgggtcaagatggaacctctggccctccagctggcgactatgagatccagacctcttcatgttccctgacacttcccctttgcatcctgtgtaatcatttcttgggccctcccaaaacctacacataaaacatacgtgatgaccattaaatagcaaaaaaaaaa

SEQ ID NO: 14 - protein, 427 aa, Mus musculus
MRRAGAACSAMDRLRLLLLLLLLLGVSFGGAKETCSTGMYTHSGECCKACNLGEGVAQPCGANQTVCEPCLDSVTFSDVVSATEPCKPCTECLGLQSMSAPCVEADDAVCRCSYGYYQDEETGRCEACSVCGVGSGLVFSCQDKQNTVCEECPEGTYSDEANHVDPCLPCTVCEDTERQLRECTPWADAECEEIPGRWITRSTPPEGSDVTTPSTQEPEAPPERDLIASTVADTVTTVMGSSQPVVTRGTADNLIPVYCSILAAVVVGLVAYIAFKRWNSCKQNKQGANSRPVNQTPPPEGEKLHSDSGISVDSQSLHDQQTHTQTASGQALKGDGNLYSSLPLTKREEVEKLLNGDTWRHLAGELGYQPEHIDSFTHEACPVRALLASWGAQDSATLDALLAALRRIQRADIVESLCSESTATSPV

SEQ ID NO: 15 - DNA, 1422 bp, Rattus norvegicus
atgaagagggcgtcctccggaggaagccggctgccgacatgggtgttatggctacaggcctggagggtagcaacgccctgccctggtgcctgtgtgtgctacaatgagcccaaggtcacaacaagccgcccccagcagggcctgcaggctgtacccgctggcatcccagcctccagccagagaatcttcctgcacggcaaccgaatctcttacgtgccagccgccagcttccagtcatgccggaatctcaccatcctgtggctgcactcaaatgcgctggccgggattgatgccgcggccttcactggtctgaccctcctggagcaactagatcttagtgacaatgcacagctccgtgtcgtggaccccaccacgttccgtggcctgggccacctgcacacgctgcacctagaccgatgcggcctgcaggagctggggcctggcctattccgtgggctggcagctctgcagtacctctacctacaagacaacaacctgcaggcacttcccgacaacaccttccgagacctgggcaacctcacgcatctctttctgcatggcaaccgtatccccagtgttcctgagcacgctttccgtggcttgcacagtcttgaccgtctcctcttgcaccagaaccatgtggctcgtgtgcacccacatgccttccgggaccttggccgactcatgaccctctacctgtttgccaacaacctctccatgctccccgcagaggtcctagtgcccctgaggtctctgcagtacctgcgactcaatgacaacccctgggtgtgtgactgcagggcacgtccgctctgggcctggctgcagaagttccgaggttcctcatccggggtgcccagcaacctaccccaacgcctggcaggccgtgatctgaagcgcctggctaccagtgacttagagggttgtgctgtggcttcggggcccttccgtcccttccagaccaatcagctcactgatgaggagctgctgggcctccccaagtgctgccagccggatgctgcagacaaggcctcagtactggaacccgggaggccggcgtctgttggaaatgcactcaagggacgtgtgcctcccggtgacactccaccaggcaatggctcaggcccacggcacatcaatgactctccatttgggactttgcccggctctgcagagcccccactgactgccctgcggcctgggggttccgagcccccgggactgcccaccacgggcccccgcaggaggccaggttgttccagaaagaaccgcacccgtagccactgccgtctgggccaggcaggaagtgggagcagtggaactggggatgcagaaggttcgggggccctgcctgccctggcctgcagccttgctcctctgggccttgcactggtactttggacagtgcttgggccctgctga

SEQ ID NO: 16 - protein, 473 aa, Rattus norvegicus
MKRASSGGSRLPTWVLWLQAWRVATPCPGACVCYNEPKVTTSRPQQGLQAVPAGIPASSQRIFLHGNRISYVPAASFQSCRNLTILWLHSNALAGIDAAAFTGLTLLEQLDLSDNAQLRVVDPTTFRGLGHLHTLHLDRCGLQELGPGLFRGLAALQYLYLQDNNLQALPDNTFRDLGNLTHLFLHGNRIPSVPEHAFRGLHSLDRLLLHQNHVARVHPHAFRDLGRLMTLYLFANNLSMLPAEVLVPLRSLQYLRLNDNPWVCDCRARPLWAWLQKFRGSSSGVPSNLPQRLAGRDLKRLATSDLEGCAVASGPFRPFQTNQLTDEELLGLPKCCQPDAADKASVLEPGRPASVGNALKGRVPPGDTPPGNGSGPRHINDSPFGTLPGSAEPPLTALRPGGSEPPGLPTTGPRRRPGCSRKNRTRSHCRLGQAGSGSSGTGDAEGSGALPALACSLAPLGLALVLWTVLGPC